(19) United States
(12) Patent Application Publication
(10) Pub. No.: US 2021/0285031 A1
(43) Pub. Date: Sep. 16, 2021

(54) FRET BASED PROTEIN BIOSENSOR FOR DETECTION OF CYCLIC DINUCLEOTIDES

(71) Applicant: University of Washington, Seattle, WA (US)

(72) Inventors: Joshua J. Woodward, Seattle, WA (US); Alex Pollock, Seattle, WA (US); Shivam Zaver, Seattle, WA (US)

(73) Assignee: University of Washington, Seattle, WA (US)

(21) Appl. No.: 17/200,503

(22) Filed: Mar. 12, 2021

Related U.S. Application Data
(60) Provisional application No. 62/990,329, filed on Mar. 16, 2020.

(51) Int. Cl. C12Q 1/6818; C07K 14/195; C07K 14/47

(52) U.S. Cl. CPC C12Q 1/6818; C07K 2319/60; C07K 14/4705; C07K 14/195

(57) ABSTRACT

The disclosure provides fusion proteins useful for detecting and quantifying cyclic dinucleotides. The fusion proteins can comprise a functional cyclic dinucleotide binding domain linked to a first fluorescent domain and a second fluorescent domain. The first fluorescent domain and a second fluorescent domain can produce a detectable signal when brought into sufficiently close proximity, such as a FRET pair or split luciferase. The fusion protein can be used to assay the presence and/or concentration of cyclic dinucleotides in vitro or in a cell. The method of use include monitoring the cyclic dinucleotide activity, or activity of cyclic dinucleotide synthases. Furthermore, the methods can include screening compounds that may modulate cyclic dinucleotide activity, production, importation, and the like.

### CROSS-REFERENCE TO RELATED APPLICATION

This application claims the benefit of U.S. Provisional Application No. 62/990,329, filed Mar. 16, 2020, the disclosure of which is incorporated herein by reference in its entirety.

### STATEMENT OF GOVERNMENT LICENSE RIGHTS

This invention was made with Government support under Grant No. R21 AI137758, awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is UWOTL173749_SeqList_final_20210308_ST25.txt. The text file is 27 KB; was created on Mar. 8, 2021; and is being submitted via EFS-Web with the filing of the specification.

### BACKGROUND

The mammalian innate immune system provides a critical first line of defense against invading microorganisms through a suite of germline encoded, invariant sentinel proteins called Pattern Recognition Receptors (PRRs). PRRs survey the extra- and intracellular milieu for molecular signatures of microbial origin, termed pathogen associated molecular patterns (PAMPs). Upon engaging PAMPs, PRRs participate in signal transduction cascades to activate antimicrobial gene regulatory programs that ultimately facilitate pathogen clearance. Microbial nucleic acids, including DNA and RNA species, constitute a major class of PAMPs. Although a number of nucleic acid sensing PRRs have been identified to-date, the enzyme cyclic GMP-AMP Synthase (cGAS) has emerged as one of the most important sensors of foreign and self double-stranded (ds) DNA.

Upon allosteric activation by dsDNA, cGAS catalyzes the production of the cyclic dinucleotide (CDN) second messenger 2′3′-cyclic GMP-AMP (c[G(2′,5′)pA(3′,5′)p], 2′3′-cGAMP, or cGAMP), which then directly binds to and activates the ER-resident, scaffold protein STING. In addition to 2′3′-cGAMP, STING also recognizes 3′3′-linked cyclic dipurines, including c-di-AMP, c-di-GMP, and 3′3′-cGAMP, produced in the context of infection with certain bacterial species, although with markedly reduced affinities. CDN-mediated activation of STING facilitates the recruitment and activation of several kinases, culminating in transcription factor-mediated cytokine expression and induction of autophagy to sterilize the cytosol of the infected cell.

Despite significant advances in the understanding of cGAS and STING regulation, the development of methods for monitoring the kinetics and dynamics of CDN signaling, especially in living cells, is limited. Most studies have relied on IFN-I induction downstream of STING activation as an indirect reporter of cGAS activity in cells. While these assays are robust and sensitive, they are not specific because many PAMPs can elicit IFN-I responses. To that end, fluorescent tagged-STING constructs have been used as a more direct reporter for STING activation. These assays rely on the translocation of STING to a perinuclear punctate compartment upon CDN binding as a qualitative proxy for its activation and have recently been employed to monitor 2′3′-cGAMP transfer via gap junctions. These tools, however, are limited to a qualitative, binary localization readout. In lieu of these reporter assays, methods to directly measure cyclic dinucleotides, including mass spectrometry, enzyme immunoassays (EIA), ENPP1-based luciferase assays (cGAMP-luc), and RNA-based biosensors have been developed. While these assays are specific, they range in their sensitivity and only provide bulk endpoint measurements following destruction of the biological sample.

Accordingly, despite the advances in the art, a need remains for facile, sensitive, and specific assays to monitor cyclic dinucleotide second messengers. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a fusion protein comprising a cyclic dinucleotide binding domain having an N terminal end and a C terminal end, a first fluorescent domain, and a second fluorescent domain.

In some embodiments, the fusion protein further comprises a first linking domain linking the first fluorescent domain and the N terminal end of the cyclic dinucleotide binding domain. In some embodiments, the first linking domain comprise from 2 to 15 amino acids, from 2 to 7 amino acids, or from 5 to 7 amino acids. In some embodiments, the fusion protein further comprises a second linking domain linking the C terminal end of the cyclic dinucleotide binding domain and the second fluorescent domain. In some embodiments, the second linking domain from 2 to 15 amino acids, from 2 to 7 amino acids, or from 5 to 7 amino acids. In some embodiments, fusion protein further comprises a first linking domain linking the first fluorescent domain and the N terminal end of the cyclic dinucleotide binding domain, wherein the fusion protein further comprises a second linking domain linking the C terminal end of the cyclic dinucleotide binding domain and the second fluorescent domain, and wherein the first linking domain comprises 7 amino acids and the second linking domain comprises 2 amino acids. In some embodiments, the fusion protein further comprises a first linking domain linking the first fluorescent domain and the N terminal end of the cyclic dinucleotide binding domain, wherein the fusion protein further comprises a second linking domain linking the C terminal end of the cyclic dinucleotide binding domain and the second fluorescent domain, and wherein the first linking domain comprises 2 amino acids and the second linking domain comprises 2 amino acids. In some embodiments, the first linking domain comprises the sequence GGSGG.

In some embodiments, the first fluorescent domain and the second fluorescent domain are complementary components of a luminescent protein. In some embodiments, the first fluorescent domain and the second fluorescent domain are complementary components of a split luciferase protein, wherein the complementary components of the split luciferase protein combine to produce a detectable signal upon binding of a cyclic dinucleotide to the cyclic dinucleotide binding domain. In some embodiments, the first fluorescent domain and the second fluorescent domain form a FRET pair. In some embodiments, the FRET pair is a blue/orange FRET pair. In some embodiments, the first fluorescent domain or the second fluorescent domain is monomeric Teal fluorescent protein (mTFP) or a derivative thereof. In some embodiments, the first fluorescent domain or the second fluorescent domain is mKO2 protein or a derivative thereof. In some embodiments, the FRET pair is a cyan/yellow FRET pair. In some embodiments, the first fluorescent domain or the second fluorescent domain is a cyan fluorescent protein (eCFP) or a derivative thereof. In some embodiments, the first fluorescent domain or the second fluorescent domain is a yellow fluorescent protein (eYFP) or a derivative thereof. In some embodiments, the FRET pair is a far-red FRET pair. In some embodiments, the fusion protein generates a FRET signal upon binding a cyclic dinucleotide to the cyclic dinucleotide binding domain. In some embodiments, the first fluorescent domain and the second fluorescent domain form a BRET pair.

In some embodiments, the cyclic dinucleotide binding domain comprises an amino acid sequence derived from a cyclic dinucleotide binding domain of a stimulator of interferon (IFN) genes (STING) protein. In some embodiments, the cyclic dinucleotide binding domain comprises an amino acid sequence derived from the cyclic dinucleotide binding domain of a murine STING (mSTING) protein. In some embodiments, the amino acid sequence has at least about 90% identity to SEQ ID NO:33. In some embodiments, the cyclic dinucleotide binding domain of the fusion protein binds a cyclic dinucleotide or ligand of a wild-type human or murine STING protein. In some embodiments, the cyclic dinucleotide is one or more of 2′,3′-cyclic GMP AMP (2′,3′-cGAMP), 3′,3′-cyclic di-AMP, 3′,3′-cyclic di-GMP, 3′,3′-cyclic GMP AMP (3′,3′-cGAMP), or a synthetic cyclic dinucleotide.

In some embodiments, the cyclic dinucleotide binding domain comprises an amino acid sequence derived from a cyclic dinucleotide binding domain of Listeria monocytogenes c-di-AMP effector protein (Lmo0553). In some embodiments, the amino acid sequence has at least about 60% identity to SEQ ID NO:35. In some embodiments, the cyclic dinucleotide is 3′3′-cyclic-di-AMP.

In another aspect, the disclosure provides a polynucleotide encoding at least one fusion protein as described herein.

In another aspect, the disclosure provides an expression cassette comprising the polynucleotide described herein operatively linked to a promoter.

In another aspect, the disclosure provides a plasmid comprising the expression cassette described herein.

In another aspect, the disclosure provides a cell comprising the polynucleotide described herein.

In another aspect, the disclosure provides a cell comprising a fusion protein described herein.

In some embodiments, the cell is a mammalian cell or bacterial cell. In some embodiments, the cell is a human cell, a primate cell, or a murine cell.

In another aspect, the disclosure provides a method for detecting a cyclic dinucleotide in a solution. The method comprises contacting the solution comprising a cyclic dinucleotide with a fusion protein as described herein for a time sufficient for the cyclic dinucleotide to bind to the cyclic dinucleotide binding domain, and detecting a signal generated by the fusion protein.

In some embodiments, the signal is a FRET signal. In some embodiments, the method comprises determining a concentration of the cyclic dinucleotide at one or more time points. In some embodiments, the solution comprises a cell extract. In some embodiments, the solution further comprises a cyclic dinucleotide synthase. In some embodiments, detecting the signal comprises performing microscopy and/or luminescence detection.

In another aspect, the disclosure provides a method for detecting a cyclic dinucleotide in a cell, comprising expressing a fusion protein described herein in a cell, and detecting a signal generated by the fusion protein. In some embodiments, the signal is a FRET signal. In some embodiments, detecting the signal comprises performing flow cytometry, microscopy, in vivo imaging, or luminescence detection. In some embodiments, the fusion protein is localized in the cytoplasm, nucleus, or plasma membrane of the cell. In some embodiments, the method comprises determining an intracellular concentration of the cyclic dinucleotide. In some embodiments, the method comprises detecting an intracellular concentration of the cyclic dinucleotide at multiple time points.

In another aspect, the disclosure provides a method for screening a component of a library for cyclic dinucleotide modulating activity, comprising contacting the component with a cell expressing a fusion protein as described herein, and detecting a signal generated by the fusion protein in the cell. In some embodiments, the signal is a FRET signal. In some embodiments, detecting the FRET signal comprising performing flow cytometry. In some embodiments, the cell comprises a cyclic dinucleotide synthase. In some embodiments, the method further comprises comparing the FRET signal generated by the fusion protein with a reference standard. In some embodiments, detecting the signal comprises performing microscopy and/or luminescence detection.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1H. BioSTING Design and Optimization. (1A) Schematic representation of full-length STING (top) and BioSTING with additional GGSGG linker between mTFP and STING-CTD (bottom). Abbreviations are defined as follows: TM (transmembrane region), DD (dimerization domain), CBD (CDN binding domain), and CTT (C-terminal tail). (1B) Overlay and structural alignment of human STING CTD in apo (“blue”) and c-di-GMP bound (“gray”) states (PDB 4F5E and 4F5D, respectively). Only a single monomer, which exhibits the largest structural rearrangement upon ligand binding, is shown. The N and C-terminal helices of the CTD are highlighted with the terminal residues in red and their associated displacement following ligand binding labeled in angstroms. (1C) Model of the FRET increase which occurs upon CDN binding. Generated with Biorender.com. (1D) Recombinant BioSTING FRET response to increasing concentrations of 2′3′-cGAMP using 458 nm excitation and listed emission wavelengths. (1E) DRaCALA radioactive nucleotide binding assay of BioSTING using ˜1 nM [³²P] labeled 2′3′-cGAMP and 3′3′-c-di-AMP. (1F) DRaCALA binding analysis of BioSTING using ˜1 nM [³²P] labeled 2′3′-cGAMP in the presence of excess (500 μM) unlabeled nucleotides. Corresponding STING-CTD competition is in FIG. 6B. (1G) Time course of excess unlabeled 2′3′-cGAMP competing off bound [³²P] labeled 2′3′-cGAMP from BioSTING and STING-CTD. (1H) Time course of excess unlabeled 3′3′-c-di-AMP competing off bound [³²P] labeled c-di-AMP from BioSTING and STING-CTD. In panels (1G) and (1H), BioSTING was pre-incubated with ˜1 nM of [³²P] labeled CDNs for 10 minutes followed by the addition of 1 mM unlabeled 2′3′-cGAMP (1G) or 3′3′-c-di-AMP (1H). In all panels, individual data points of n=2 biological replicates are shown.

FIGS. 2A-2F. Real time measurement of CDN synthesis and determination of CDN levels from cellular extracts. (2A) DisA enzyme activity assay time course in the presence of increasing concentrations of the 3′3′-c-di-AMP cyclase DisA using BioSTING. (2B) cGAS activity assay in the presence of indicated concentrations of recombinant cGAS with or without Interferon Stimulatory DNA (ISD) using BioSTING. (2C) cGAS activity assay measuring 2′3′-cGAMP production in the presence of fixed cGAS and ISD concentrations with increasing concentrations of the cGAS inhibitor PF-06928215 using BioSTING. (2D) Reaction linear rates calculated from panel (2E) plotted versus PF-06928215 concentration and fit to IC50 curve (Prism 8). (2E) BioSTING FRET response in the presence of methanol extracted cGAMP from HEK293T cells transfected with increasing concentrations of pCDNA3.1-cGAS. (2F) BioSTING FRET response over two-fold serial dilutions of cGAMP methanol extracts from HEK293T cells transfected with 5 μg of pCDNA3.1-cGAS. In panels (2A) and (2E), individual data points of n=2 biological replicates are shown. In panels (2B)-(2D) and (2F), data are presented as mean±s.d. of n=3 (2B, 2F) or n=8 (2C, 2D) biological replicates.

FIGS. 3A-3H. BioSTING quantitates cGAMP in single cells in a manner compatible with flow screening. HEK293T cells stably expressing BioSTING transfected with 1 μg of either empty pCDNA3.1 vector or pCDNA3.1-cGAS vector and analyzed for (3A) cGAS expression by western blot (data are representative of two independent experiments), (3B) cGAMP production by EIA analysis, and (3C) BioSTING FRET response by flow cytometry. (3D) HEK293T cells stably expressing BioSTING were electroporated using AMAXA (Lonza) with increasing concentrations of purified 2′3-cGAMP and analyzed for FRET response by flow cytometry. (3E) HEK293T cells stably expressing BioSTING were transfected with increasing concentrations of pCDNA3.1-cGAS and analyzed for FRET response by flow cytometry or for 2′3′-cGAMP production by EIA. (3F) BioSTING FRET response from (3E) graphed as a function of intracellular 2′3′-cGAMP concentration measured by EIA. (3G-3H) HEK293T cells stably expressing BioSTING transfected with either empty-pCDNA3.1 vector or pCDNA3.1-cGAS vector and analyzed by the alternative gating method in S2d for (3G) cells in the low gate and (3H) cells in the high gate. Statistical analyses were performed using two-tailed t-tests: *** denotes P=0.0006 (3G) and **** denotes P=0.0000002 (3H) (Prism 8). In panels (3B, 3C) and (3G, 3H), data are presented as mean±s.d. of n=4 biological replicates. In panels (3D-3F), individual data points of n=2 biological replicates are shown.

FIGS. 4A-4E. BioSTING variants exhibit distinct specificity for metazoan and bacterial CDNs. (4A) Interactions made by human STING residues Y240 (mouse Y239) and T263 (mouse T262) hypothesized to stabilize CDN binding in PDB 4F5D visualized in PyMol. (4B) DRaCALA binding analysis of WT and Y239S/T262A BioSTING using [³²P] labeled 2′3′-cGAMP. (4C) Recombinant WT and Y239S/T262A BioSTING FRET response in the presence of increasing concentrations of 2′3′-cGAMP. Data are presented as mean±s.d. of n=4 biological replicates. (4D) HEK293T cells stably expressing WT or Y239S/T262A BioSTING were transfected with increasing concentrations of pCDNA3.1-cGAS and analyzed for FRET response by flow cytometry. (4E) HEK293T cells stably expressing WT or Y239S/T262A BioSTING were transfected with increasing concentrations (125, 250, 500, or 1000 ng) of expression vectors for cGAS, DisA, WspR*, or empty vector and analyzed for FRET response by flow cytometry. Data in panels (4B), (4D) and (4E) are presented as individual data points of n=2 biological replicates are shown.

FIGS. 5A-5G. BioSTING exhibits broad utility for monitoring diverse aspects of cGAMP signaling. (5A) HEK293T cells stably expressing BioSTING were transfected with 10 ng of pCDNA3.1-cGAS for 16-20 hours then transfected again with an increasing concentration of activating CT-DNA for 4 hours and analyzed by flow cytometry. (5B) HEK293T cells stably expressing BioSTING were transfected with 3 μg of WT or H17A Poxin and increasing concentrations of pCDNA3.1-cGAS and analyzed for FRET response by flow cytometry. (5C) Increasing concentrations of 2′3′-cGAMP were added to the media of HEK293T cells stably expressing BioSTING for 6 hours and analyzed for FRET response by flow cytometry. (5D-5E) BioSTING expression was induced in HEK293T cells transduced with pSLIK-BioSTING or pSLIK-NLS-BioSTING for 24 hours by the addition of doxycycline and biosensor expression was analyzed using a BZ-X710 microscope (Keyence) using brightfield and a GFP filter cube (EX 470/40, DM 495, and BA 525/50) with a 40× objective. Scale bars, 15 μm. Data are representative of two independent experiments. (5F) HEK293T cells stably expressing untagged or NLS-tagged WT or Y239S/T262A BioSTING were transfected with increasing concentrations of pCDNA3.1-cGAS and analyzed for FRET response by flow cytometry. (5G) HEK293T cells stably expressing untagged WT BioSTING were transfected with 100 ng of cGAS expression plasmid or empty vector alone with either 100 ng of empty vector or vector expressing full-length (FL) human STING and analyzed by flow cytometry. In all panels, individual data points of n=2 (5A-5C, 5F, and 5G) or n=3 (5G) biological replicates are shown.

FIGS. 6A and 6B. BioSTING development and characterization. (6A) FRET response in the presence of increasing 2′3′-cGAMP of prototype biosensor (no linker) and biosensors with GGSGG linkers on the N, C), or both termini of STING-CTD. (6B) DRaCALA binding analysis of STING-CTD using ˜1 nM [32P] labeled 2′3′-cGAMP and 3′3′-c-di-AMP.

FIGS. 7A-7D. BioSTING detection of CDNs in vitro. (7A) cGAS activity assay in the presence and absence of indicated concentrations of recombinant cGAS, ISD, ATP, and GTP using BioSTING. (7B) Determination of Z′ factor and signal to noise (S/N) ratio for BioSTING using recombinant cGAS in the presence and absence of ISD in a 96-well format. 0.5<Z′<1 is considered excellent statistical reliability for high throughput screening applications. (7C) cGAS activity assay measuring 2′3′-cGAMP production in the presence of a fixed concentration of PF-06928215 (500 μM) with increasing concentrations of cGAS using BioSTING. (7D) Quantification of 2′3′-cGAMP levels from HEK293T cells transfected with 5 μg of pcDNA3.1-cGAS using BioSTING or EIA. In panels (7A) and (7B), data are presented as mean±s.d. of n=3 (7A) or n=9 (7B) biological replicates. In panels 7C and 7D, individual data points of n=2 (7C, 7D) or n=3 (7D) biological replicates are shown.

FIGS. 8A-8D. Crystal structure of Lmo0553 in complex with c-di-AMP. (8A) Schematic drawing of the Lmo0553 dimer bound to two c-di-AMP (cdA) molecules. The CBS motifs and the ACT domain of one monomer are labelled as “cyan” and “dark blue”, respectively, and those of the other monomer as “green” and “dark green”. (8B) Schematic drawing of the Lmo0553 dimer bound to two c-di-AMP (cdA) molecules, viewed after a 900 rotation around the vertical axis. (8C) Detailed interactions between Lmo0553 and c-di-AMP. The first and second nucleotides of c-di-AMP are labeled 1 and 2, respectively. Hydrogen-bonding interactions are shown as a dashed line. (8D) Conformational changes in the c-di-AMP binding site. Overlay of the structure of Lmo0553 in complex with c-di-AMP with that of free Lmo0553. All structure figures were produced with PyMOL.

FIGS. 9A-9E. Binding c-di-AMP induces a large structural change in Lmo0553 guiding development of CDA5. (9A) Overlay of the structure of Lmo0553 in complex with c-di-AMP with that of free Lmo0553. The Bateman domain of one monomer (“cyan”) is overlaid, in order to visualize the changes in the position of the other domains in the dimer (“green”). The conformational change in the ACT domain is indicated by the arrow. (9B) Same as panel 9A, but viewed after a 90° rotation around the vertical axis. The conformation change in the CBS domain is indicated by the arrow. (9C) Model of the restructuring used to generate the CDA5 biosensor (9D). Recombinant CDA5 FRET response to increasing concentrations of c-di-AMP using 425 nm excitation and 480 nm and 535 nm emission wavelengths. Data presented as individual n=1 data points. (9E) Schematic of CDA5 FRET increase upon c-di-AMP binding. Panels 9A and 9B were produced with PyMOL. Panels C and E were produced using RioRender.

FIGS. 10A-10F. CDA5 retains native and physiologically relevant binding characteristics. (10A) DRaCALA radioactive nucleotide binding assay of CDA5 and full length Lmo0553 using ˜1 nM [³²P] labeled 3′3′-c-di-AMP. Data fit to a nonlinear curve. Radioactive c-di-AMP bound Lmo0553 and CDA5 at 4.83 μM and 5.87 μM respectively. (10B) DRaCALA radioactive nucleotide binding assay of CDA5 using ˜1 nM [³²P] labeled 3′3′-c-di-AMP in the presence of excess (500 μM) unlabeled nucleotides. (10C) HEK293T cells stably expressing CDA5 were transfected with increasing concentrations of pCDNA4-DisA and analyzed for FRET response by flow cytometry. (10D) HEK293T cells stably expressing CDA5 were transfected with 2000 ng of expression vectors for DisA, cGAS, WspR*, or empty vector and analyzed for FRET response by flow cytometry. (10E) DRaCALA radioactive nucleotide binding assay time course of CDA5 and full length Lmo0553 using ˜1 nM [³²P] labeled 3′3′-c-di-AMP in the presence of excess (500 μM) unlabeled c-di-AMP. (10F) PdeA phosphodiesterase activity assay time course in the presence of decreasing concentrations of recombinant PdeA (2-fold dilutions from 160 μM) monitored using CDA5. 2 μM c-di-AMP was re-spiked into the solution at 90 minutes. In panels 10A-10E, data are presented as n=3 biological replicates. Panel 10F is presented as individual n=1 data points connected by a line.

FIGS. 11A-11D. CDA5 Y34A is an important c-di-AMP blind control. (11A) DRaCALA radioactive nucleotide binding assay of CDA5 WT and Y34A using ˜1 nM [³²P] labeled 3′3′-c-di-AMP. (11B) Recombinant CDA5 WT and Y34A FRET response to increasing concentrations of c-di-AMP. (11C) BL21 (DE3) E. coli transformed with pET15b-CDA5 (WT or Y34A) and pBAV-dacA or empty vector then analyzed by flow cytometry. (11D) Data in panel 11C converted into Y/A ratio. Data in panel 11A is presented as n=3 biological replicates. Panel 11B is presented as individual n=1 data points. Data in panels 11C and 11D are presented as n=2 biological replicates.

FIGS. 12A-12G. CDA5 allows for the detection of native c-di-AMP dynamics in single cells. (12A) B. subtilis expressing CDA5 WT and Y34A back diluted into 10% LB media and grown at 37° C. and analyzed for FRET response by flow cytometry (12B) CDA5 WT FRET ratio in panel A plotted versus c-di-AMP quantitated by mass spectrometry. (12C) CDA5 Y/A ratio in panel A plotted versus c-di-AMP quantitated by mass spectrometry. (12D-12G) CDA5 WT FRET ratio of single cells divided by the average CDA5 Y34A FRET ratio plotted as histograms at indicated time points. Data in panels 12A-12C are presented as mean and standard deviation of n=3 biological replicates. Data in panels 12D-12G are presented as histograms consisting of individual data points of n=2 replicates. Histograms respectively labelled to represent CDA5 Y34A and CDA5 WT.

FIGS. 13A-13C. CDA5 detects varied c-di-AMP dynamics in B. subtilis mutant. (13A) WT, ΔpgpH, and ΔdisA B. subtilis back diluted into 10% LB media and grown at 37° C. and analyzed for FRET response by flow cytometry presented as Y/A ratios (13B-13C) CDA5 WT FRET ratio of single cells divided by the average CDA5 Y34A FRET ratio plotted as histograms at indicated time points. Data in all panels are presented as individual data points of n=2 biological replicates.

FIG. 14. CBS domain structural rearrangement upon c-di-AMP binding. Movie models the rearrangement of Lmo0553 upon c-di-AMP binding. “Yellow” and “Cyan” denote location of eYFP and eCFP fusion respectively. Made using PyMol.

### DETAILED DESCRIPTION

Cyclic dinucleotides (CDNs) are second messengers conserved across all three domains of life. Within eukaryotes they mediate protective roles in innate immunity against malignant, viral, and bacterial disease and exert pathological effects in autoimmune disorders. Despite their ubiquitous role in diverse biological contexts, CDN detection methods are limited. This disclosure presents a platform of facile, sensitive, and flexible reagents and methods to detect, quantify, and monitor levels of CDNs using conditional fluorescent signal (i.e., when a target CDN binds to a fusion protein construct with affinity for the CDN). The disclosure is based on the inventors design and implementation of fusion proteins that can emit a detectable signal upon binding of a target CDN. The fusion proteins can be used in vitro or expressed in vivo for detection in living cells.

As described in more detail in Example 1, the inventors first conducted a structure-guided design of the murine STING CDN binding domain, and engineered a Förster resonance energy transfer (FRET) based biosensor, referred to as “BioSTING”. In test assays, the recombinant BioSTING permitted real-time detection of CDN synthase activity and inhibition. Expression of BioSTING in live human cells allowed quantification of localized bacterial and eukaryotic CDN levels in single cells with low nanomolar sensitivity. These findings establish BioSTING as a powerful kinetic in vitro platform amenable to high throughput screens and as a broadly applicable cellular tool to interrogate the temporal and spatial dynamics of CDN signaling in vivo in a variety of infectious, malignant, and autoimmune contexts.

Furthermore, 3′3′-cyclic di-adenosine monophosphate (c-di-AMP) is an important nucleotide second messenger found throughout the bacterial domain of life. C-di-AMP is essential in many bacteria and regulates a diverse array of effector proteins controlling pathogenesis, cell wall homeostasis, osmoregulation, and central metabolism. Despite the ubiquity and importance of c-di-AMP, methods to detect this signaling molecule are limited, particularly at single cell resolution. As described in more detail in Example 2, crystallization of the Listeria monocytogenes c-di-AMP effector protein Lmo0553 enabled structure-guided design of a Förster resonance energy transfer (FRET) based biosensor, which is referred to as “CDA5”. CDA5 is a fully genetically encodable, specific, and reversible biosensor that allows for the detection of c-di-AMP dynamics both in vitro and within live single cells in a nondestructive manner. The initial studies identified a unimodal distribution of c-di-AMP in Bacillus subtilis that decreased rapidly when cells were grown in diluted Luria Broth. Furthermore, it was demonstrated that B. subtilis mutants lacking either a c-di-AMP phosphodiesterase or cyclase had respectively higher and lower FRET responses, again in a unimodal manner. These findings new provide insight into c-di-AMP distribution within bacterial populations and establish CDA5 as a novel and powerful platform for characterizing new aspects of c-di-AMP regulation.

Fusion Protein

In accordance with the foregoing, in one embodiment the disclosure provides a fusion protein comprising a cyclic dinucleotide (CDN) binding domain having an N terminal end and a C terminal end, a first fluorescent domain, and a second fluorescent domain.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide or protein is specifically intended to cover naturally occurring proteins, as well as those that are recombinantly or synthetically produced. The term “fusion” protein refers to a protein that contains at least two peptide or polypeptide domains or subsequences that do not naturally occur together and, thus, the fusion protein is artificial in the sense that human intervention is required to produce it. The fusion protein can be synthesized or produced recombinantly according to techniques known in the art. For example, recombinant expression comprises the arrangement of nucleic acid sequences encoding the two or more peptide or polypeptide sequences in a single expression cassette such that encoded domains are expressed together and in-frame in the final fusion protein. The expression cassette can be introduced into a cell or other expression system, e.g., using an expression vector, to facilitate transcription and translation of the fusion protein.

The CDN binding domain is a polypeptide domain that has binding affinity for a CDN and, when bound to a CDN, the CDN binding domain exhibits a change in spatial (e.g., tertiary) structure. More detail on exemplary CDN binding domains is provided below. The first fluorescent domain and second fluorescent domain are arranged in the fusion protein relative to the CDN binding domain in a manner that facilitates changes in spatial positioning when a CDN is bound to the CDN binding domain compared to when no CDN is bound to the CDN binding domain. This change of spatial positioning depending on the binding state of the CDN binding domain leads to a change in detectable signal because the first fluorescent domain and the second fluorescent domain are moved closer to or further from each other in a manner that alters a detectable signal.

In some embodiments, the first fluorescent domain is linked, directly or indirectly, to the N terminal end of the CDN binding domain. In some embodiments, the second fluorescent domain is linked, directly or indirectly, to the C terminal end of the CDN binding domain. In some embodiments, the first fluorescent domain is linked, directly or indirectly, to the N terminal end of the CDN binding domain and the second fluorescent domain is linked, directly or indirectly, to the C terminal end of the CDN binding domain. One or both of the first fluorescent domain and the second fluorescent domain can be linked or fused directly to CDN binding domain.

In some embodiments, one or both of the first fluorescent domain and the second fluorescent domain are linked the CDN binding domain indirectly via a linker, e.g., a flexible peptide or polypeptide linker. Suitable linkers or “linking domains” are generally those that allow each of the linked domains of the fusion protein to fold with a three-dimensional structure very similar to the structure of the domains when produced individually (e.g., without other components attached thereto). The linking domain can be relatively rich in small nonpolar amino acid residues, polar amino acid residues, and/or hydrophilic amino acid residues, such as, for example, glycine, serine, and threonine (see, e.g., Bird R et al, Science 242: 423-6 (1988); Friedman P et al, Cancer Res. 53: 334-9 (1993); and Siegall C et al, J Immunol 152: 2377-84 (1994), each of which is incorporated herein by reference in its entirety).

For example, in some embodiments, the fusion protein further comprises a first linking domain linking the first fluorescent domain and the N terminal end of the CDN binding domain. The first linking domain can comprise 1, 2, 3, 4, 5, 6, 7, 8, 9, 10, 11, 12, 13, 14, 15, 16, 17, 18, 19, or 20 amino acids. In some embodiments, the first linking domain comprises from 2 to 15 amino acids, from 2 to 7 amino acids, or from 5 to 7 amino acids. In other embodiments, regardless of the presence of a first linking domain, the fusion protein further comprises a second linking domain linking the second fluorescent domain and the C terminal end of the CDN binding domain. The second linking domain can comprise 1, 2, 3, 4, 5, 6, 7, 8, 9, 10, 11, 12, 13, 14, 15, 16, 17, 18, 19, or 20 amino acids. In some embodiments, the second linking domain comprises from 2 to 15 amino acids, from 2 to 7 amino acids, or from 5 to 7 amino acids. In some embodiments, the fusion protein comprises a first linking domain and a second linking domain, as described above. In some embodiments, the first linking domain comprises the sequence GGSGG (SEQ ID NO: 28).

To illustrate, in one exemplary embodiment the fusion protein further comprises a first linking domain linking the first fluorescent domain and the N terminal end of the CDN binding domain, and a second linking domain linking the C terminal end of the CDN binding domain and the second fluorescent domain, wherein the first linking domain comprises 7 amino acids and the second linking domain comprises 2 amino acids. In a further embodiment, this arrangement exists with the dinucleotide binding domain being, or being derived from, a STING CDN binding domain (e.g., murine-derived STING), described in more detail below.

In another exemplary embodiment, the fusion protein further comprises a first linking domain linking the first fluorescent domain and the N terminal end of the cyclic dinucleotide binding domain, and a second linking domain linking the C terminal end of the cyclic dinucleotide binding domain and the second fluorescent domain, wherein the first linking domain comprises 2 amino acids and the second linking domain comprises 2 amino acids. In a further embodiment, this arrangement exists with the dinucleotide binding domain being, or being derived from, an Lmo0553 CDN binding domain, described in more detail below.

The first fluorescent domain and the second fluorescent domain can operate as complementary components of a luminescent construct, such that when arranged in an appropriate configuration with respect to each other, the first and second fluorescent domains cooperate to emit a distinct detectable signal (e.g., light of a distinguishable wavelength) compared to when the first and second fluorescent domains are not arranged in the appropriate configuration. In some embodiments, the first and second fluorescent domains cooperate to emit detectable light when they are in the appropriate configuration compared to providing no emission of light when the first and second fluorescent domains are not arranged in the appropriate configuration. In other embodiments, one or both of the first and second fluorescent domains can emit light without interaction with the other fluorescent domain, but when they are properly configured, they cooperate to emit light at a different wavelength.

Appropriate proteinaceous moieties or molecules that can serve as the first and second fluorescent domains are known and are encompassed by this disclosure. In some embodiments, the first fluorescent domain and the second fluorescent domain are complementary components of a luminescent protein. When the complementary components are brought into appropriate configuration, i.e., by the conformational changes induced by binding of a CDN to the CDN binding domain, the first fluorescent domain and the second fluorescent domain interact and function together as the luminescent protein. An example of such luminescent protein is a luciferase that is split into the first fluorescent domain and the second fluorescent domain by the CDN binding domain. With the conformational shift induced by CDN binding, the first fluorescent domain and the second fluorescent domain can interact resulting a change in emitted signal. See, e.g., Azad T, et al. Split-luciferase complementary assay: applications, recent developments, and future perspectives. Anal Bioanal Chem. 406(23):5541-60 (2014), incorporated herein by reference in its entirety.

In another embodiment, the first fluorescent domain and the second fluorescent domain form a Förster resonance energy transfer (FRET) pair. FRET is a mechanism whereby energy is transferred between two light-sensitive molecules (chromophores or fluors). In a FRET pair, typically one member of the pair serves as a donor that, in an excited state, transfers energy to the second member of the pair, which serves as the acceptor, through dipole-dipole coupling. The transfer of energy is detected by virtue of a unique wavelength of light that is released. The efficiency of the energy transfer is extremely sensitive to spatial distance and so the emission of the unique signal is only detected when the two pair members are in very close and precise proximity.

In these embodiments, the first fluorescent domain and the second fluorescent domain of the fusion protein are a donor/acceptor pair, where one (the first or second fluorescent domain) being the donor and the other (the second or the first fluorescent domain, respectively) being acceptor. Appropriate FRET pairs serving as the first fluorescent domain and the second fluorescent domain in the present fusion protein construct are widely known and can be selected for implementation depending on the application or use of the fusion protein (e.g., based on the desired wavelengths for excitation and detection, etc.). For example, see Bajar, B. T., et al. A Guide to Fluorescent Protein FRET Pairs. Sensors (Basel). 16(9):1488 (2016), incorporated herein by reference in its entirety, which discusses the FRET concept and discloses many exemplary FRET fluor pairs that are encompassed by this disclosure. Exemplary categories of FRET pairs encompassed by this disclosure include cyan/yellow fluorescent proteins, green/red fluorescent proteins, far-red/infrared fluorescent proteins, large Stokes shift (LSS)-based fluorescent proteins, Dark fluorescent protein-based pairs, optical highlighter fluorescent proteins (i.e., phototranformable fluorescent proteins (ptFPs)), multicolor FRET pairs, and homo-FRET pairs.

In one illustrative embodiment, the FRET pair is a blue/orange FRET pair. For example, one of the pair members (i.e., the first fluorescent domain or the second fluorescent domain) can be monomeric Teal fluorescent protein (mTFP) or a functional derivative thereof. A representative mTFP protein has the sequence set forth as SEQ ID NO:29. Additionally or alternatively, one of the pair members (i.e., the first fluorescent domain or the second fluorescent domain) can be mKO2 protein or a derivative thereof. A representative mKO2 protein has the sequence set forth as SEQ ID NO:30 In some embodiments, one of the pair members (e.g., the first fluorescent domain) is mTFP or a functional derivative thereof (e.g., as set forth in SEQ ID NO:29), whereas the other pair member (e.g., the second fluorescent domain) is mKO2 protein or a derivative thereof (e.g., as set forth in SEQ ID NO:30). A person of ordinary skill in the art would readily understand that the roles of mTFP and mKO2 as the first and second fluorescent protein can be reversed within the fusion protein without loss of functionality.

In another illustrative embodiment, the FRET pair is a cyan/yellow FRET pair. For example, one of the pair members (i.e., the first fluorescent domain or the second fluorescent domain) can be cyan fluorescent protein (eCFP) or a functional derivative thereof. A representative eCFP protein has the sequence set forth as SEQ ID NO:31. Additionally or alternatively, one of the pair members (i.e., the first fluorescent domain or the second fluorescent domain) is yellow fluorescent protein (eYFP) or a derivative thereof. A representative eYFP protein has the sequence set forth as SEQ ID NO:32. In some embodiments, one of the pair members (e.g., the first fluorescent domain) is cyan fluorescent protein (eCFP) or a functional derivative thereof (e.g., as set forth in SEQ ID NO:31), whereas the other pair member (e.g., the second fluorescent domain) is yellow fluorescent protein (eYFP) or a derivative thereof (e.g., as set forth in SEQ ID NO:32). As above, a person of ordinary skill in the art would readily understand that the roles of eCFP and eYFP as the first and second fluorescent protein can be reversed within the fusion protein without loss of functionality.

In another illustrative embodiment, the FRET pair is a red-shifted or far-red FRET pair, which can offer more flexibility with respect to expanded ranges of optogenetic tools. Exemplary fluors that can be incorporated into a far-red FRET pair as encompassed by this disclosure are disclosed in Piatkevich, K. D. and Verkhusha, V. V. Guide to Red Fluorescent Proteins and Biosensors for Flow Cytometry. Methods Cell Biol. 102: 431-461 (2011), incorporated herein by reference in its entirety, and are encompassed by the present disclosure. In some embodiments, the FRET pair comprises one or both of mKOκ and mKate2. See, e.g., Watabe, T., et al. Booster, a Red-Shifted Genetically Encoded Förster Resonance Energy Transfer (FRET) Biosensor Compatible with Cyan Fluorescent Protein/Yellow Fluorescent Protein-Based FRET Biosensors and Blue Light-Responsive Optogenetic Tools. ACS Sens. 5(3):719-730 (2020), incorporated herein by reference in its entirety.

In another embodiment, the first fluorescent domain and the second fluorescent domain form a bioluminescence resonance energy transfer (BRET) pair. BRET is similar to FRET, but incorporates a bioluminescent donor instead of a fluorescent donor. A distinction with FRET is that the FRET donor must be excited, e.g., with an external light source, to initiate the energy transfer to the acceptor, whereas a bioluminescent donor does not require an external light source. BRET donor moieties or proteins are known and encompassed by the present disclosure and can be readily incorporated into a pairing appropriate for a BRET signal. BRET donor moieties or proteins can require a substrate to induce the initial bioluminescence. An exemplary BRET donor moiety or protein is a luciferase enzyme.

In some embodiments, the first fluorescent domain and the second fluorescent domain form the functional FRET or BRET pair upon binding of a CDN to the CDN binding domain and, in contrast, cease emission of the detectable signal when a CDN is not bound to the CDN binding domain.

The CDN binding domain can be derived from any known protein that binds to CDN. In one embodiment, the CDN binding domain is derived from the CDN domain of a stimulator of interferon (IFN) genes (STING) protein. STING is an ER membrane protein that binds to a variety of CDNs. STING proteins and their CDN binding domains are known from across a wide variety of organisms, from mammals (e.g., human or murine STING) to invertebrate (e.g., sea anemone), all of which are encompassed by this disclosure. Particularly advantageous are mammalian STING proteins, which can bind CDNs of both mammalian (eukaryotic) and bacterial (prokaryotic) origin. A non-limiting example of a CDN binding domain encompassed by this disclosure is the CDN binding domain from a murine STING protein (mSTING). An exemplary sequence of a mSTING CDN binding domain is set forth in the SEQ ID NO:32. Accordingly, in some embodiments, the CDN binding domain comprises the amino acid sequence set forth in SEQ ID NO:33, or an amino acid sequence with at least about 80%, at least about 85%, at least about 90%, at least about 95%, at least about 98% sequence identity to SEQ ID NO:32, or a CDN binding fragment of any of the foregoing domain sequences, which can be readily tested.

It is noted that STING proteins homologs do not exhibit a high degree of amino acid sequence conservation but do retain structural and functional features and, as such are identifiable by persons of ordinary skill in the art.

For example, certain conserved domains (e.g., between the mouse and sea anemone sequence) point to conservation of structure to provide critical CDN binding functionality. Thus, in some embodiments, the fusion protein CDN binding domain comprises one or more of the following subdomains:

an amino acid sequence with at least 85% identity to residues 2-21 of SEQ ID NO:33;

an amino acid sequence with at least 70% identity to residues 86-95 of SEQ ID NO:33;

an amino acid sequence with at least 75% identity to residues 105-120 of SEQ ID NO:33; and

an amino acid sequence with at least 55% identity to residues 128-148 of SEQ ID NO:33.

In further embodiments, for any of the subdomains indicated above, a majority (e.g., about 50%, about 60%, about 70%, or more) of the residues that do not exhibit direct sequence identity are conservative substitutions of the corresponding residues in reference SEQ ID NO:33. CDN-binding domains of STING proteins from prokaryotic and eukaryotic sources are known and are also encompassed by the present disclosure as other embodiments of the CDN binding domain of the disclosed fusion protein. Exemplary STING proteins from which the CDN binding domain of the present fusion protein can be derived include: human (6DNK_A), Orcinus orca (XP_033262553.1), Bos mutus (MXQ91008.1), Acropora millepora (XP_029195486.1)), and the like. Each of these exemplary CDNs are incorporated herein by reference and encompassed by the disclosure.

In some embodiments, the CDN binding domain, regardless of origin, can bind to a CDN or a natural ligand of a human or murine STING protein. This functionality can be readily tested by persons of ordinary skill in the art. In some embodiments, the CDN binding domain of the fusion protein binds to a CDN. The CDN can be naturally occurring or synthetic. Illustrative, non-limiting examples include one or more of 2′,3′-cyclic GMP AMP (2′,3′-cGAMP), 3′,3′-cyclic di-AMP, 3′,3′-cyclic di-GMP, 3′,3′-cyclic GMP AMP (3′,3′-cGAMP), or non-naturally occurring modifications thereof. In some embodiments, the CDN binding domain binds to a non-naturally occurring or synthetic CDN, such as 2′3′-RR-CDA (referred to as ADU-S100).

In a specific embodiment, the first fluorescent domain is derived from mTFP or mKO2, the CDN binding domain is derived from mSTING, and the second binding domain is derived from the other of mKO2 or mTFP. For example, such an embodiment can comprise an amino acid sequence set forth in SEQ ID NO:34, or a sequence with at least about 84%, at least about 86%, at least about 88%, at last about 90%, at least about 92%, at least about 94%, at least about 96%, or at least about 98% identity thereto.

In another embodiment, the CDN binding domain is derived from the CDN domain of a c-di-AMP effector protein from a Listeria organism. An exemplary c-di-AMP effector protein is Lmo0553 from Listeria monocytogenes. While the function of Lmo0553 is currently unresolved, it is known to bind to c-di-AMP. The protein is discussed in more detail in Example 2. A domain referred to as the Bateman domain, which interacts with c-di-AMP. Accordingly, in some embodiments the fusion protein CDN binding domain is derived from the Lmo0553 Bateman domain or corresponding domains in homologs of Lmo0553. An exemplary Bateman domain of Lmo0553 has the amino acid set forth in SEQ ID NO:35. Accordingly, in some embodiments the fusion protein CDN domain comprises an amino acid sequence as set forth in SEQ ID NO:35 or a sequence with at least about 70%, at least about 75%, at least about 80%, at least about 85%, at least about 90%, at least about 95%, or at least about 98% identity thereto.

In some embodiments, the Bateman domain is derived from Listeria sp. or homologous proteins in Enterococcus sp. including Enterococcus sp. 94-2 (WP_137599739.1, incorporated herein by reference in its entirety)

In further embodiments, the fusion protein with the Bateman domain binds 3′3′-cyclic-di-AMP.

In a specific embodiment, the first fluorescent domain is derived from eYFP or eCFP, the CDN binding domain is derived from lmo0553, and the second binding domain is derived from the other of eCFP or eYFP. For example, such an embodiment can comprise an amino acid sequence set forth in SEQ ID NO:36, or a sequence with at least about 84%, at least about 86%, at least about 88%, at last about 90%, at least about 92%, at least about 94%, at least about 96%, or at least about 98% identity thereto.

In some embodiments, the fusion protein described herein is capable of homodimerization and the resulting dimers bind to one or more CDNs

The fusion protein can comprise additional sequences or domains that facilitate additional functionalities. For example, the fusion protein can comprise a tag to facilitate detection or purification of the fusion protein. For example, tag can be a histidine tag at the N or C terminus of the fusion protein. Other such tags are known. In some embodiments, the fusion protein further comprises a target peptide sequence that can guide the transport of the fusion protein to a specific target area in the cell. Target peptide sequence are short peptide sequences that are typically present on proteins at the N-terminus and facilitate transport of the expressed protein to various specific targets within the cell. A variety of different target peptide sequence are known and can be readily selected and incorporated by skilled practitioners to promote transport of the protein to organelles or locations of choice within the cell. For example, the fusion protein can further comprise a nuclear localization sequence (NLS) for transport to the nucleus. Alternatively, the fusion protein can comprise a target sequence that facilitate delivers to the mitochondria, endoplasmic reticulum (ER), chloroplast, apoplast, peroxisome, plasma membrane, and the like.

Nucleic Acids and Related Constructs

In another aspect, the disclosure provides a nucleic acid molecule encoding any of the fusion proteins described herein. For example, a person of ordinary skill in the art can use the genetic code to determine nucleic acid sequences that can encode fusion proteins comprising a cyclic dinucleotide binding domain having an N terminal end and a C terminal end, a first fluorescent domain, and a second fluorescent domain, based on the above disclosures.

In another aspect, the disclosure provides an expression cassette comprising the nucleic acid further operatively linked to a promoter sequence. The term “promoter” refers to a regulatory nucleotide sequence that can activate transcription (expression) of a gene and/or splice variant isoforms thereof. A promoter is typically located upstream of a gene, but can be located at other regions proximal to the gene, or even within the gene. The promoter typically contains binding sites for RNA polymerase and one or more transcription factors, which participate in the assembly of the transcriptional complex. As used herein, the term “operatively linked” indicates that the promoter and the encoding nucleic acid are configured and positioned relative to each other a manner such that the promoter can activate transcription of the encoding nucleic acid by the transcriptional machinery of the cell. The promoter can be constitutive or inducible. Constitutive promoters can be determined based on the character of the target cell and the particular transcription factors available in the cytosol. A person of ordinary skill in the art can select an appropriate promoter based on the intended cell for expression, as various promoters are known and commonly used in the art.

In some embodiments, the disclosure provides a vector comprising the nucleic acid described above. The vector can be any construct that facilitates the delivery of the nucleic acid to the target cell and/or expression of the nucleic acid within the cell. The vectors can be viral vectors, circular nucleic acid constructs (e.g., plasmids), or nanoparticles (solid or lipid-based).

Plasmids are circular nucleic acid constructs that typically comprise the expression cassette, as described above, in addition to other sequence components to facilitate functionality, such as a gene encoding antibiotic resistance, origin of replication, restriction sites, and the like. A great variety of plasmids are known and are encompassed by this disclosure.

Various viral vectors are also known in the art and are encompassed by the present disclosure. See, e.g., Machida, C. A. (ed.), Viral Vectors for Gene Therapy: Methods and Protocols, Humana Press, Totowa, N.J. (2003); Maczka, N., (ed.), Current Topics in Microbiology and Immunology: Viral Expression Vectors, Springer-Verlag, Berlin, Germany (2012), each incorporated herein by reference in its entirety. In some embodiments, the viral vector is an adeno associated virus (AAV) vector, an adenovirus vector, a retrovirus vector, or a lentivirus vector.

Cells

In another aspect, the disclosure provides a cell comprising the nucleic acid encoding any fusion protein as described herein. In some embodiments, the cell comprises a vector, wherein the vector comprises the nucleic acid encoding any fusion protein as described herein. The cell is capable of expressing the fusion protein from the nucleic acid. For example, the nucleic acid and/or vector can be configured for expression of the fusion protein from the encoding nucleic acid within the cell. A promoter operatively linked to the nucleic acid can be appropriately configured to allow binding of the cell's RNA polymerase and one or more transcription factors to permit assembly of the transcriptional complex.

The disclosure encompasses any type of cell for this aspect, eukaryotic or prokaryotic.

Methods

As indicated herein, the disclosed fusion peptide can generate a detectable signal when bound to a CDN. Accordingly, the fusion proteins are useful for detection and quantification of CDNs in vivo or in vitro. Such detection and quantification can provide valuable information regarding the production or importation of CDN second messengers.

Accordingly, in another aspect, the disclosure provides a method for detecting a cyclic dinucleotide in a solution. The method comprises contacting the solution potentially comprising a cyclic dinucleotide with a fusion protein described herein for a time sufficient for the cyclic dinucleotide to bind to the cyclic dinucleotide binding domain. The fusion protein will produce a detectable signal upon binding and, thus, the method comprises detecting the signal.

The signal can be a fluorescence, e.g., FRET, signal. Detection of the signal can be performed by microscopy and/or forms of luminescence detection, according to known techniques.

The method can comprise determining the intensity or frequency of the signal and, therefore, determining the concentration of the CDN in the solution. The detection can occur at multiple time points. The solution can comprise a cell extract, e.g., an extract from a cell engineered to express the fusion protein.

In certain embodiments, the solution comprises a cyclic dinucleotide synthase. The method can be used to determine the rate of production of the CDNs by the cyclic dinucleotide synthase. This can be used to screen compounds for their effects on the activity of the cyclic dinucleotide synthase. The method can be scaled up to perform a plurality of assays in parallel, e.g., in a multi-well plate, to screen a library of compounds for effects on the activity of one or more cyclic dinucleotide synthase.

In another aspect, the disclosure provides a method for detecting a cyclic dinucleotide in a cell. The method comprises expressing the fusion protein described herein in the cell and detecting a signal generated by the fusion protein. As above, the signal can be a fluorescent signal, such as a FRET signal. The detection can be accomplished by performing flow cytometry, microscopy, in vivo imaging, or luminescence detection, according to known methods. As indicated above, some embodiments of the fusion protein can have signal peptides and, thus, the detectable signal can be detected or tracked into specific subcellular compartments, such as the cytoplasm, nucleus, plasma membrane, Golgi apparatus, etc.

The methods can comprise determining an intracellular concentration of the CDN based on the detected signal. Furthermore, the method can comprise detecting the intracellular concentration over time.

The method can be used to applying experimental conditions to the cell to may affect the production, importation, or longevity of CDN, and then track the concentration of the CDN over time. Such methods can further characterize the roles of CDNs as second messengers in response to various conditions, and to test compositions that can modulate the CDN-based signaling.

In yet another aspect, the disclosure provides a method of screening a component for CDN modulating activity. The method comprises contacting the component with a cell expressing a fusion protein described herein, and detecting a signal generated by the fusion protein in the cell. As above, the signal can be a fluorescence signa, e.g., FRET signal. The techniques for detection can include, e.g., flow cytometry, microscopy, in vivo imaging, or luminescence detection, according to known methods.

The cell can comprise a cyclic dinucleotide synthase. The activity of the cyclic dinucleotide synthase can be assay in the screen, where modulation of the cyclic dinucleotide synthase activity is determined.

In some embodiments of any of the methods described herein, the methods further comprises comparing the detected signal to a reference standard appropriate for the experimental design.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

As used herein, the term “nucleic acid” refers to any polymer molecule that comprises multiple nucleotide subunits (i.e., a polynucleotide). Nucleic acids encompassed by the present disclosure can include deoxyribonucleotide polymer (DNA), ribonucleotide polymer (RNA), cDNA or a synthetic nucleic acid known in the art.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:

(1) Alanine (A), Serine (S), Threonine (T),

(2) Aspartic acid (D), Glutamic acid (E),

(3) Asparagine (N), Glutamine (Q),

(4) Arginine (R), Lysine (K),

(5) Isoleucine (I), Leucine (L), Methionine (M), Valine (V), and

(6) Phenylalanine (F), Tyrosine (Y), Tryptophan (W).

Reference to sequence identity addresses the degree of similarity of two polymeric sequences, such as protein or nucleic acid sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

This Example describes the development of one embodiment of the fusion construct for detecting CDNs, described above. Specifically, this Example describes the development of a Förster resonance energy transfer (FRET)-based biosensor, referred to BioSTING, that can provide real-time detection of CDN synthase activity and inhibition in vitro and in vivo. BioSTING is demonstrated to be a powerful kinetic in vitro platform amenable to high throughput screens and as a broadly applicable cellular tool to interrogate the temporal and spatial dynamics of CDN signaling in a variety of infectious, malignant, and autoimmune contexts. This investigation was published as Pollock, A. J., Zaver, S. A., & Woodward, J. J. A STING-based biosensor affords broad cyclic dinucleotide detection within single living eukaryotic cells. Nature Communications, 11(1), 1-13 (2020), which is incorporated herein by reference in its entirety.

Introduction

FRET biosensors have been developed for the detection of various small molecules. Ligand binding to an intramolecular FRET biosensor results in a conformational shift that alters the relative distance and orientation of fused compatible donor and acceptor fluorophores. This, in turn, alters the excitation energy transfer between fluorophores, which can be quantitated by exciting the donor fluorophore and determining the ratio of acceptor fluorophore emission to donor fluorophore emission. This change in fluorescence is directly linked to ligand occupancy and thus reports on ligand concentration either in solution or within cells. Because these biosensors are based on native ligand binding proteins, they are powerful, genetically encodable tools with biologically relevant binding affinities and responses. Use of these biosensors has provided fundamental insight into the spatial and temporal dynamics of nucleotide signaling as well as the identification of activators and inhibitors following in vitro and cellular screens.

To address the limitations of current CDN detection techniques, an intramolecular FRET biosensor, referred to as BioSTING, was developed based on the eukaryotic CDN binding protein STING (Huang, Y.-H., et al. The structural basis for the sensing and binding of cyclic di-GMP by STING. Nature Structural & Molecular Biology 19, 728-730 (2012), incorporated herein by reference in its entirety). It is demonstrated in this example that recombinant BioSTING is a sensitive tool capable of detecting real-time CDN synthesis, drug inhibition, and extracted CDNs from cellular sources. Further, it is shown that BioSTING expressed in eukaryotic cells can be used to detect CDN synthesis, import, localization, and degradation by a viral protein. Thus, BioSTING affords a powerful in vitro and cellular platform for monitoring CDN levels and is can facilitate fundamental discoveries relating to CDN biology as well as translational drug discovery campaigns.

Results

Design and Development of BioSTING

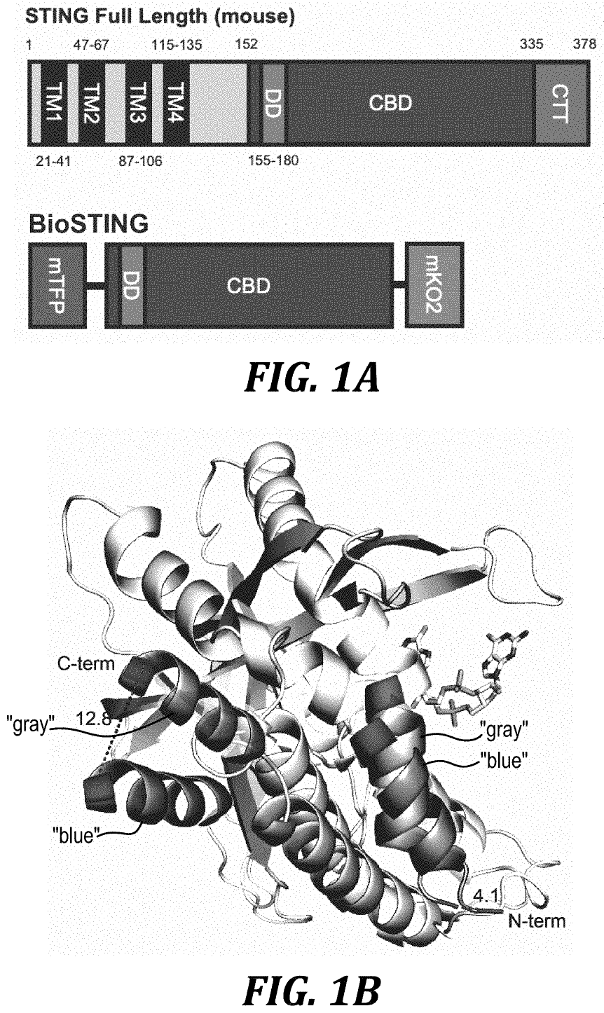

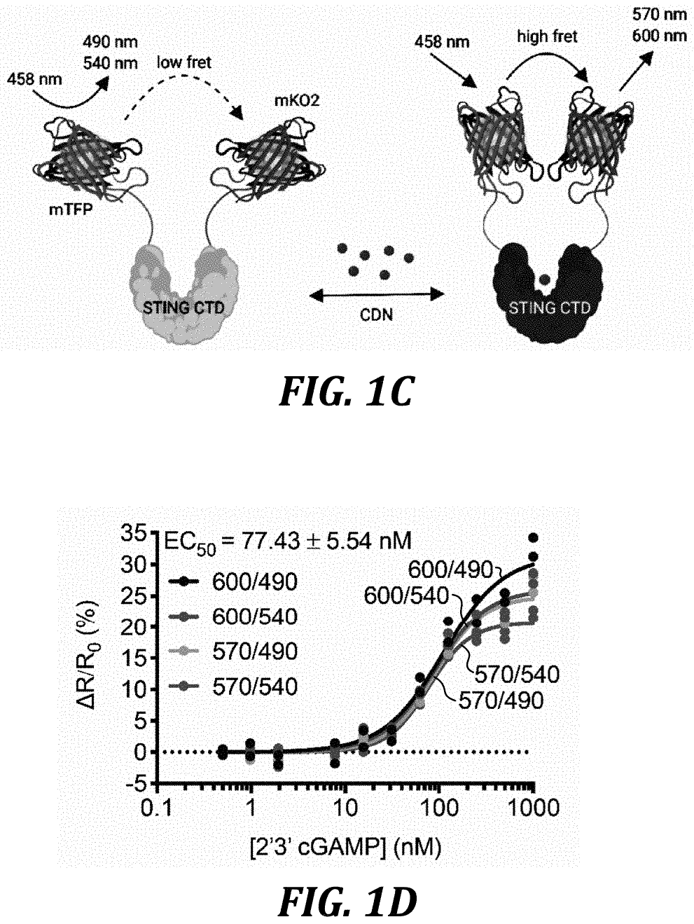

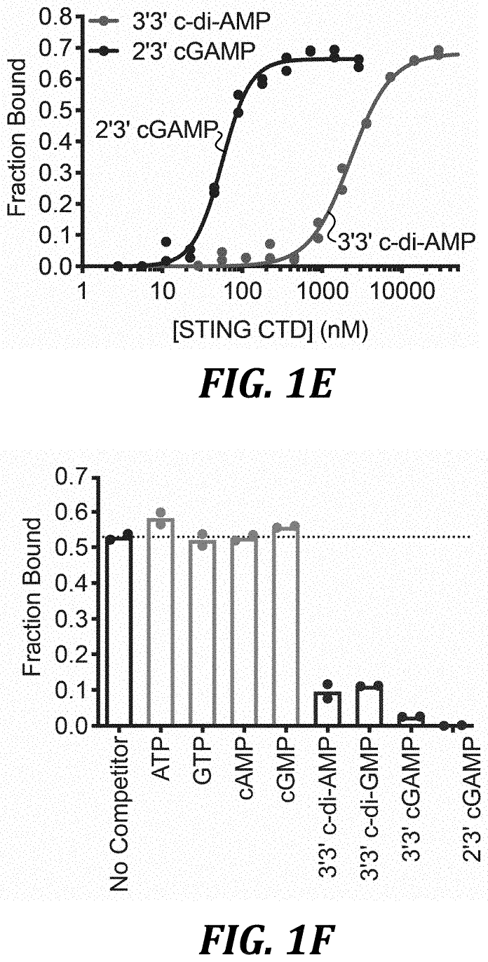

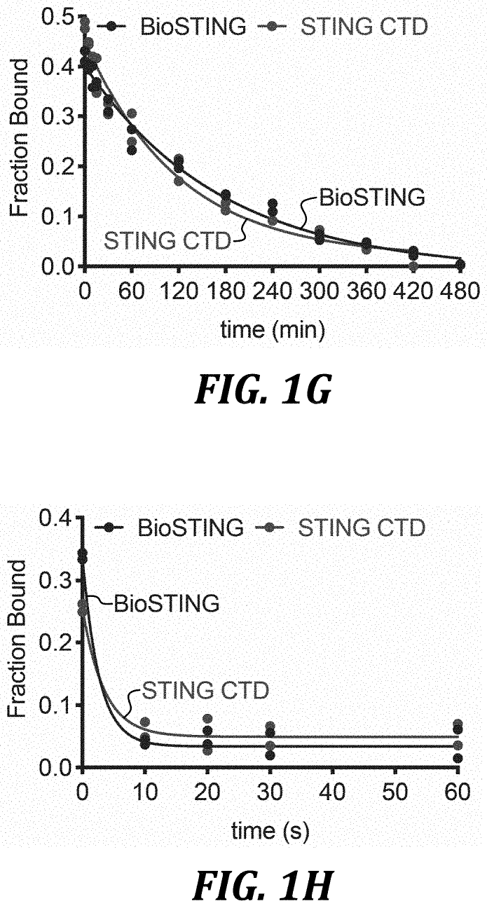

CDN signal transduction is mediated by nucleotide binding to effector proteins. Ligand induced structural changes in the protein alter effector function to execute changes in response to altered cellular concentrations of the second messenger. The mammalian protein STING is unique in its ability to bind a variety of CDNs of both mammalian and bacterial origin. Structurally, STING is a multipass membrane protein with a C-terminal domain consisting of a dimerization region, a CDN binding domain, and a C-terminal tail (CTT) that is required for downstream signaling (FIG. 1A, top panel). The CDN binding domain has been extensively characterized at atomic resolution. When the structures of human STING in the apo and c-di-GMP bound states are aligned, one monomer in each dimer was observed to overlap with nearly no change. However, the second monomer exhibited translocation of over 4 and 12 angstroms in the N and C-termini, respectively (FIG. 1B). Because FRET is highly sensitive to the intermolecular distance between compatible fluorophores, the question of whether a fluorescent fusion protein with the STING CTD would afford a platform for the development of a FRET-based CDN biosensor was investigated (FIG. 1C).

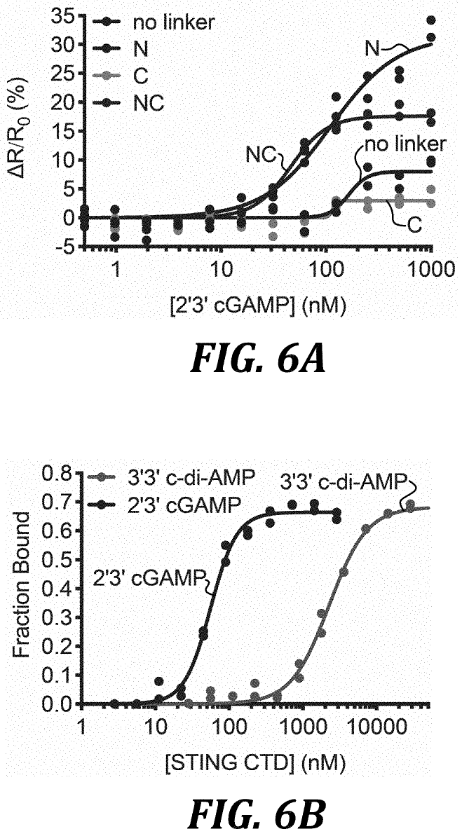

Unlike some human STING alleles, murine STING (mSTING) binds both bacterial and eukaryotic dipurine containing CDNs. Therefore, to construct a reporter of broad utility, a prototype sensor was generated by fusing mSTING to the bright and photostable FRET pair mTFP and mKO2 at L152 and E335, respectively (FIG. 1A, bottom panel). The residues contained within this region of mSTING include the dimerization domain and the CDN binding domain but exclude the amino-terminal transmembrane domains and CTT. Based on analysis of the crystal structure, it was hypothesized that, upon cyclic dinucleotide binding, the C-terminus fused to mKO2 would shift in closer proximity to mTFP and increase the amount of FRET signal (FIG. 1C). Indeed, a modest ˜7% FRET increase was observed in the presence of cGAMP with purified recombinant protein (FIG. 6A). While this observation demonstrated the utility of this approach, the FRET signal of this prototype was thought to be amenable to optimization to enhance the FRET signal. Notably, no FRET change was observed using the eCFP and eYFP FRET pair, highlighting the importance of fluorophore orientation as a factor in generating a FRET response.

FRET is exquisitely sensitive to changes in distance and orientation. As such, a five amino acid GGSGG linker was added between mSTING-CTD and each fluorophore individually and in combination. While addition of the linker between STING-CTD and mKO2 slightly diminished the FRET response, addition of GGSGG between mTFP and STING-CTD alone successfully increased the FRET change to over 20% (FIG. 1D, TABLE 1, and FIG. 6A). This modified FRET biosensor responded to 2′3′-cGAMP with a dynamic range of 12-125 nM and a limit of detection (signal-to-noise ratio 3:1) of approximately 12 nM (TABLE 1). Because a 20% FRET increase is more than sufficient for cellular applications, this protein was named “BioSTING” (Biosensor STING) and this version was used in all further experiments described below.

TABLE 1
BioSTING parameters.
BioSTING Parameters

| | |
|---|---|
| Kd (nM) | 56 |
| EC50 (nM) | 77.4 |
| L.C.D. (nM) | 12.6 |
| L.O.Q. (nM) | 38.2 |
| Dynamic Range (nM) | 12-125 |
| ΔFRET Ratio (%) | ~25 |

Characterization of BioSTING-CDN Binding

Having generated BioSTING, the next step was to compare the biochemical parameters of nucleotide interactions between purified BioSTING and STING-CTD by DRaCALA analysis (Roelofs, K. G., et al. Differential radial capillary action of ligand assay for high-throughput detection of protein-metabolite interactions. Proceedings of the National Academy of Sciences 108, 15528-15533 (2011), incorporated herein by reference in its entirety). Radioactive cGAMP bound BioSTING and STING-CTD at a Kd of 56 and 61 nM, respectively (FIGS. 1E and 6B). Radioactive c-di-AMP bound BioSTING and STING-CTD at a Kd of 2.26 μM and 2.58 μM, respectively (FIGS. 1E and 6B). These results confirmed that the region of STING contained within BioSTING maintained CDN binding and that the addition of flanking FRET-fluorophores did not alter binding affinity or disrupt the dimer to which CDNs bind. To confirm that BioSTING retained binding specificity, radioactive cGAMP binding analysis was performed with both BioSTING and STING-CTD in the presence of a variety of unlabeled nucleotides. As expected, only 3′3′- and 2′3′-cyclic dinucleotides but not ATP, GTP, cAMP, or cGMP could compete off bound [³²P] 2′3′-cGAMP (FIG. 1F). These findings reveal that fusion of the two FRET fluorophores to BioSTING has no discernable impact on nucleotide specificity or binding affinity relative to STING-CTD.

Finally, the dissociation kinetics were monitored by determining the rate that cold nucleotide could compete off bound radioactive nucleotide. Radioactive 2′3′-cGAMP was competed off BioSTING and STING-CTD by cold 2′3′-cGAMP with a half-life of 121 and 83 minutes, respectively (FIG. 1G). Radioactive 3′3′-c-di-AMP was competed off of both proteins with a half-life of <10 seconds by cold 3′3′-c-di-AMP (FIG. 1H). Together, these results show similar dissociation rates of CDNs from BioSTING relative to STING-CTD, consistent with the similarity in the observed Kd's for these nucleotides. Additionally, due to the rapid dissociation rate of 3′3′-c-di-AMP, BioSTING is anticipated to afford rapid monitoring of both increases and decreases in nucleotide levels of 3′3′-CDNs. However, as a consequence of the relatively slow dissociation rate of 2′3′-cGAMP, BioSTING may afford real time monitoring of increases in 2′3′-cGAMP but may be limited in the temporal resolution associated with its decline. Notably, these results suggest that STING activation by 2′3′-cGAMP may be possible through a single binding event while activation by 3′3′-CDNs may require constant exposure to an activating concentration of ligand.

BioSTING Provides a Real-Time Readout of Cyclic Dinucleotide Production In Vitro

Current in vitro methods for monitoring CDN production employ sensitive EIA, mass spectrometry, and cGAMP-luc endpoint measures, as well as less sensitive continuous tools, including a cGAMP RNA biosensor and an indirect pyrophosphate release assay. Given the limitations of current CDN detection methods, the ease of recombinant BioSTING production, and its capacity to directly report on a variety of CDNs at biologically relevant concentrations, BioSTING was employed to monitor in vitro CDN synthase activity and inhibition to demonstrate its utility for kinetic characterization and feasibility for high throughput screening related to these enzymes.

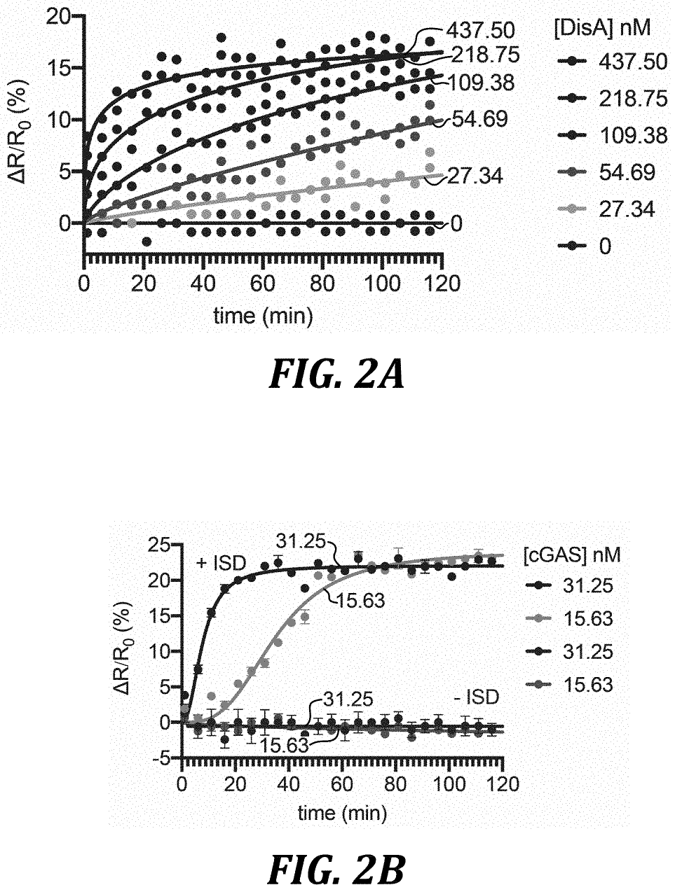

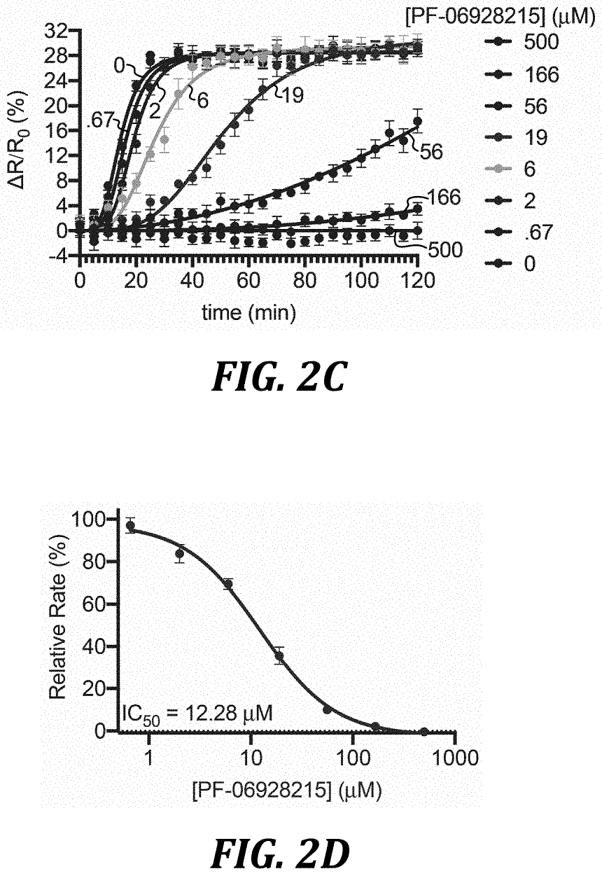

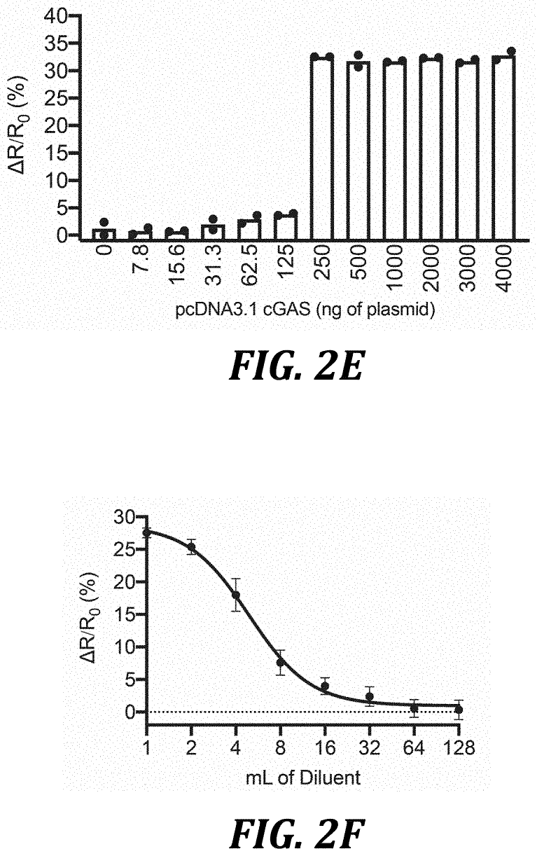

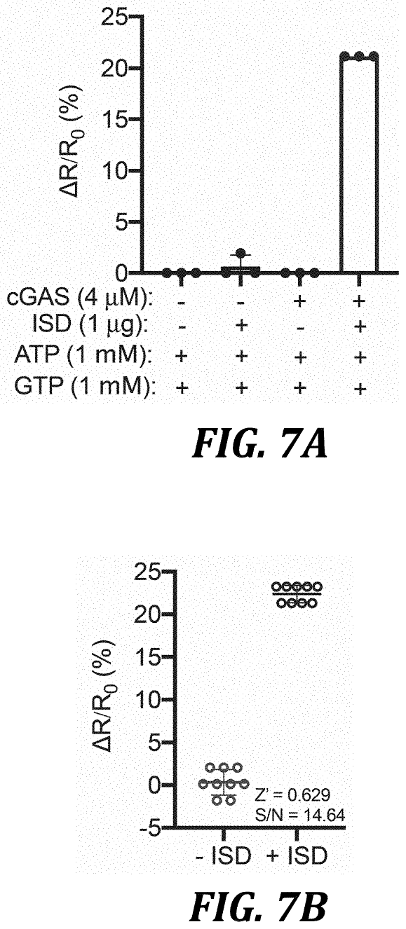

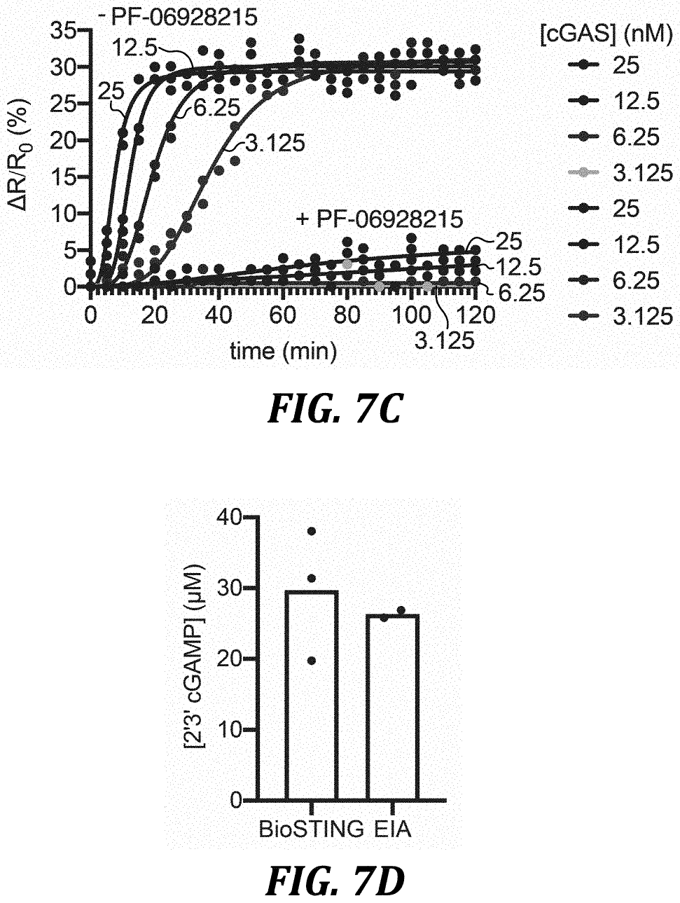

To demonstrate the ability of recombinant BioSTING to detect real time production of cyclic dinucleotides, DNA integrity scanning protein A (DisA) mediated synthesis of 3′3′-c-di-AMP, which occurs constitutively in the presence of ATP, was monitored. In reactions consisting of BioSTING, ATP, and increasing concentrations of purified DisA, increasing rates of FRET signal concomitant with increasing DisA concentrations were observed (FIG. 2A). Next, cGAS synthesis of 2′3′-cGAMP from ATP and GTP was monitored. As expected, the rate of BioSTING FRET signal correlated with increased cGAS protein and there was no 2′3′-cGAMP production without the addition of DNA, which is required for allosteric activation of the enzyme (FIGS. 2B and 7A).

BioSTING FRET assays are carried out in a 96-well format using a fluorescent plate reader making it amenable to in vitro high throughput screening efforts (FIG. 7B). Recently, Pfizer reported the development of the cGAS inhibitor PF-06928215. To test the use of BioSTING as a platform to characterize small molecule inhibitors, dose response measures were performed with fixed cGAS and variable concentrations of PF-06928215 (FIG. 2C). In parallel, cGAS levels were also titrated while keeping the concentration of PF-06928215 fixed (FIG. 7C). The slope of the linear region was determined for each reaction, plotted versus PF-06928215 concentration, and fit to identify an IC50 of ˜12 μM (FIG. 2D), in close agreement with the reported IC50 of 5 μM. Together these studies demonstrate that BioSTING affords robust, continuous detection of CDN levels in vitro and offers flexibility in assay design, making it amenable to high throughput screens and kinetic characterization studies of a variety of CDN synthases. In addition to screening for modulators of CDN synthase activity, BioSTING could also potentially be used to screen directly for STING agonists and antagonists.

BioSTING can Detect 2′3′-cGAMP Extracted from Mammalian Cells

Given the ease of producing recombinant BioSTING and its nanomolar affinity for 2′3′-cGAMP, it provides an attractive method for measuring 2′3′-cGAMP from cellular extracts. To demonstrate this utility, FRET responses of BioSTING exposed to methanol extracts from HEK293T cells transfected with increasing concentrations of pCDNA3.1-cGAS were monitored. As expected, FRET responses were negligible with low concentrations of transfected pCDNA3.1-cGAS plasmid but saturated at elevated plasmid concentrations (FIG. 2E). Notably, at lower levels of transfected plasmid marginal FRET responses were observed. This is likely a consequence of the simultaneous increase in both cGAS expression and activating DNA as well as the dilution factor chosen for the experiment. To overcome this limitation, samples containing low levels of cGAMP can be diluted less or subjected to a cGAMP enrichment step using recombinant STING.

Additionally, signal saturation with higher levels of transfected plasmid was observed. To overcome signal saturation, samples transfected with the highest amount of pCDNA3.1-cGAS were diluted to obtain readings within the sensor's linear range (FIG. 2F). Multiplying the dilution factor by the diluted sample's cGAMP concentration calculated by interpolation into a standard curve of known cGAMP concentrations (FIG. 1D), thus allows for determination of cGAMP in the undiluted sample. If cell volume assumptions are applied, an average intracellular cGAMP concentration can be calculated, in this case ˜30 μM, which was in line with the concentration determined by EIA and within the range of concentrations previously reported by other methods. Together, these findings suggest that recombinant BioSTING can be used as a bulk cell extract 2′3′-cGAMP detection method.

BioSTING can Detect 2′3′-cGAMP within Live Mammalian Cells

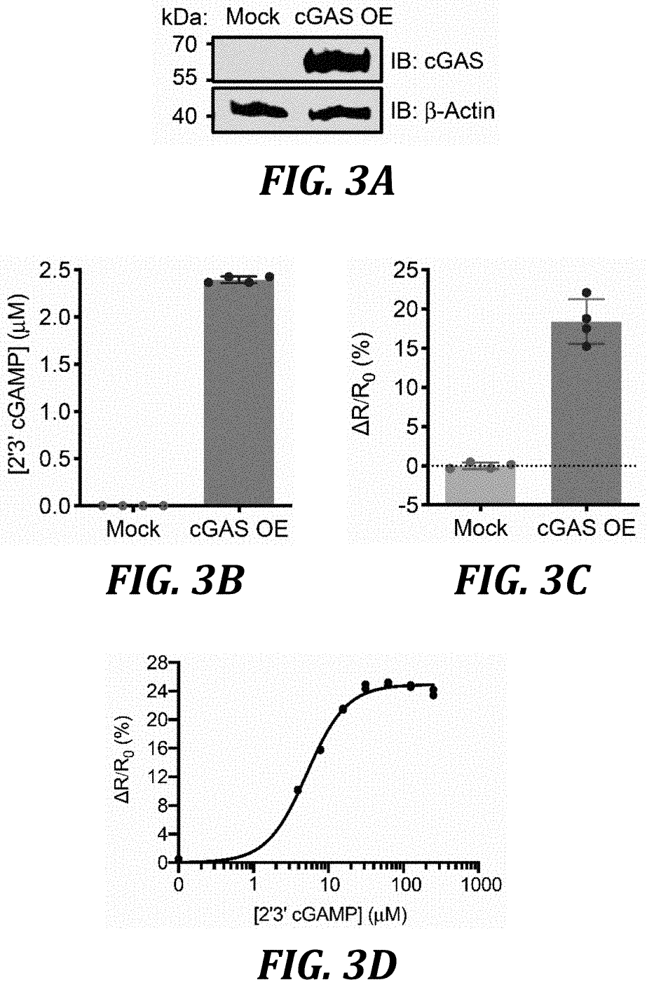

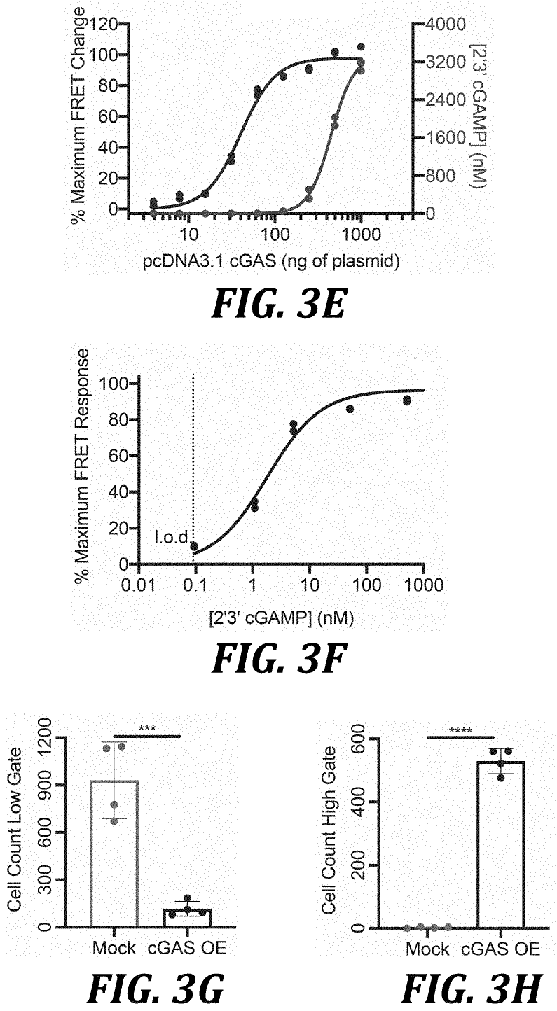

The ability to directly monitor cyclic dinucleotides in individual living eukaryotic cells is currently one of the biggest limitations in the field. Encouraged by the in vitro characterization of BioSTING, application was next extended within this context. BioSTING was introduced into a pSLIK doxycycline inducible lentiviral system to generate stable HEK293T cell lines. Briefly, BioSTING expression was induced in HEK293T cells transduced with pSLIK-BioSTING for 24 hours after the addition of doxycycline or vehicle control. Biosensor expression was analyzed using a BZ-X710 microscope (Keyence), GFP filter cube (EX 470/40, DM 495, and BA 525/50), and 40× objective (not shown). Fixed levels of either pCDNA3.1-cGAS or empty vector were transfected into cells expressing BioSTING and FRET signal was monitored using flow cytometry. In cells expressing cGAS relative to an empty vector control, a greater than 20% BioSTING FRET increase was observed, consistent with the in vitro assays (FIGS. 3A-3E,). The flow analysis method included: Step 1—set a permissive size gate, Step 2 and 3—sort for single cells using FSC and SSC A by H plots, and step 4—plot BV510 (mTFP) by PE (mKO2) and gate on cells with appropriate expression. The cells are then analyzed for BV570 (FRET)/BV510 (CFP) ratio. This method was used to collect data for all flow analysis unless otherwise noted. Additionally, production of 2′3′-cGAMP was confirmed in this experimental system by EIA analysis (FIG. 3B). To ensure that BioSTING FRET responses were directly due to cGAMP recognition, purified cGAMP was delivered using both lipofection and nucleofection. Lipofectamine transfection produced a strong response, which plateaued below the BioSTING saturation level, likely as a consequence of the limited carrying capacity of the transfection reagent (not shown). Briefly, HEK293T cells stably expressing BioSTING were transfected with either pCDNA3.1-cGAS or increasing concentrations of purified 2′3′-cGAMP using Lipofectamine 2000 transfection reagent according to the manufacturer's protocol and analyzed for FRET response by flow cytometry. Nucleofection, on the other hand, was not limited in this manner, and complete BioSTING activation was observed (FIG. 3D).

As intracellular cGAMP concentrations can range from low nanomolar to high micromolar, to quantitatively contextualize the observed BioSTING FRET response in cells, the levels of cGAS expression vector were titrated and the intracellular concentration of cGAMP was determined by EIA in parallel with BioSTING FRET measurements by flow cytometry. Using assumptions about cell volume, FRET changes were plotted versus average intracellular cGAMP concentration from which it was estimated that 50% of the maximum BioSTING FRET signal corresponds to a concentration of approximately 5-50 nM cGAMP in cells, consistent with the in vitro measured affinity of BioSTING for cGAMP and supporting the expectation that BioSTING is responding to biologically relevant concentrations of cGAMP (FIGS. 3E and 3F).

Reanalysis of the flow cytometry data to detect FRET levels of single cells rather than the entire population confirmed that, under the conditions tested, there was a unimodal rather than bimodal shift in signal (not shown). Briefly, single cell analysis was performed on HEK293T cells stably expressing BioSTING transfected with 10 ng of pCDNA3.1-cGAS. The cells were then transfected with a mock (0 ng), medium (156 ng), or high (1250 ng) amounts of cGAS-activating CT-DNA for 4 hours and analyzed by flow cytometry. Because the entire population is responding with a normal distribution, the possibility of using BioSTING as a platform for screening via flow cytometry was interrogated. To estimate sorting potential, a FRET high gate was drawn above cells transfected with an empty vector and a FRET low gate drawn below cells transfected with a high amount of pCDNA3.1-cGAS (not shown). ˜10-fold enrichment was obtained for cells in the FRET low gate (FIG. 3G) and ˜100 fold enrichment was obtained for cells in the FRET high gate (FIG. 3H). It is hypothesized that the decreased selection in the FRET low gate is a consequence of a small percentage of cells evading efficient transfection. Thus, efficiency of selection could likely be improved using an integrated inducible or constitutive cGAS system. Overall, these encouraging results provide support for utilizing BioSTING as a platform for forward genetic screens to identify genes involved in regulating cGAS and cGAMP in living cells.

In order to assess the impacts of BioSTING expression levels on intracellular FRET responses, the flow cytometry results were again reanalyzed to compare cells expressing high and low levels of BioSTING. The cells were first analyzed as described above and then separated as low fluors and high fluors (not shown). These two populations had similar FRET response dynamics but with slightly different response magnitudes (not shown). Thus, although there is flexibility in the expression level of cells, BioSTING expression levels should be tightly controlled when comparing FRET responses between samples.

Development and Use of a cGAMP-Blind BioSTING

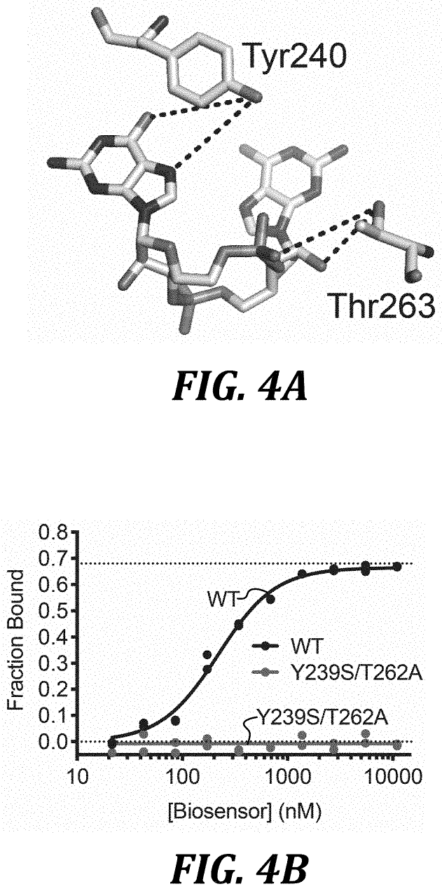

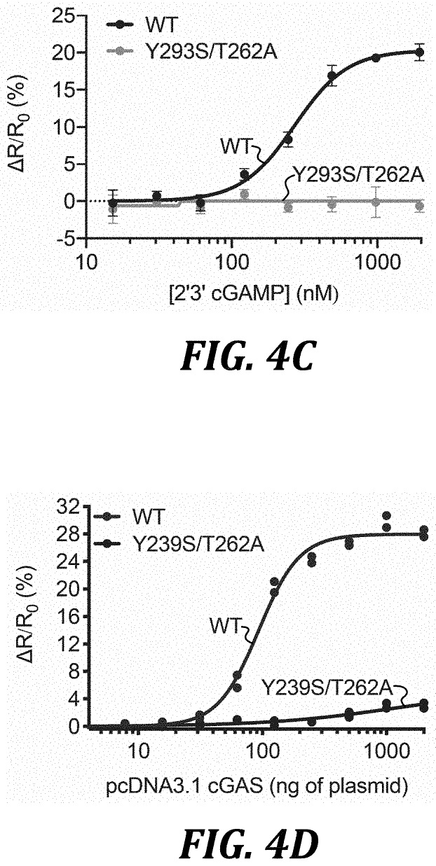

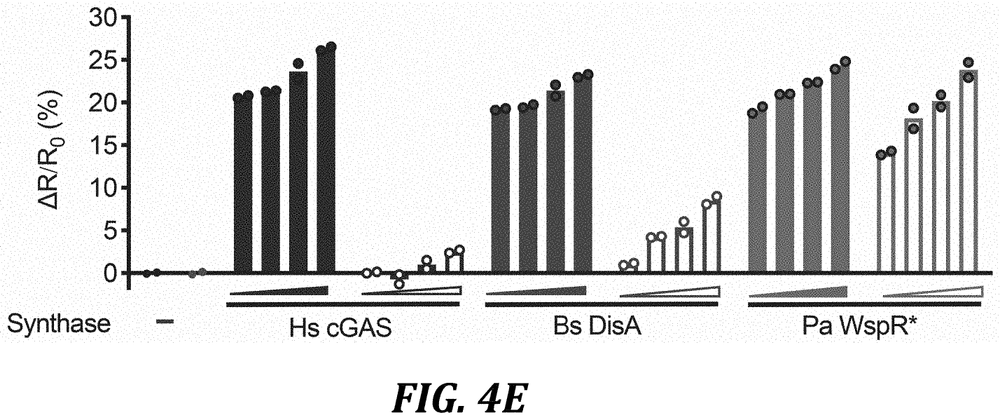

A powerful control for an intramolecular FRET biosensor is a ligand blind version that differentiates changes due to bonafide binding from other effects (i.e. protein-protein interactions, fluorophore quenching, etc.). A literature search highlighted the mutations Y240S and T263A in human STING (Y239S and T262A in murine STING) as ideal candidates which would diminish CDN binding without disrupting protein stability (FIG. 4A). As expected, Y239S T262A BioSTING stably expressed and neither appreciably bound radioactive cGAMP nor produced a FRET change in response to cGAMP (FIGS. 4B and 4C). To determine if Y239S T262A BioSTING could act as a cGAMP control in cells, pCDNA3.1-cGAS was titrated in cells expressing WT or mutant biosensor. While WT BioSTING produced a robust FRET response, Y239S T262A BioSTING only generated a minor FRET change at exceptionally high levels of cGAS, thus supporting the use of Y239S T262A BioSTING as a control biosensor for 2′3′-cGAMP (FIG. 4D).

Bacterial 3′3′-CDNs have been shown to be released during infection and to activate STING in the cytosol. To determine if BioSTING and Y239S T262A BioSTING can detect bacterial CDNs in cells, increasing concentrations of expression vectors for B. subtilis DisA, which synthesizes c-di-AMP, and a constitutively active isoform of P. aeruginosa WspR (D70E), denoted WspR*, which synthesizes c-di-GMP, were titrated (FIG. 4E). All nucleotides resulted in a BioSTING FRET increase. However, while Y239S T262A BioSTING produced a minor response to the highest concentrations of cGAS, moderate responses were observed for DisA, and unexpectedly large responses were observed for WspR*. Levels of c-di-AMP are not expected to reach high levels in physiologically relevant contexts, therefore Y239S T262A BioSTING is likely to serve as an adequate control for c-di-AMP. In contrast, the lowest levels of WspR* tested induced a large response in Y239S T262A BioSTING demonstrating that it will not be an adequate control for c-di-GMP. Thus, despite its applicability as a control for other CDNs, the Y239S T262A BioSTING variant may be selectively used as a c-di-GMP biosensor within cells.

It was hypothesized that further design may be used to generate other versions of BioSTING that exhibit selectivity for CDNs based upon their unique chemical properties, including phosphodiester linkage and/or base content. Previous studies identified naturally occurring mutations in human STING that abolish responsiveness to 3′3′-CDNs while retaining 2′3′-cGAMP sensing. In an effort to engineer a universally blind control biosensor, the previously reported R231A/H mutations were implemented to diminish IFN-0 activation in Y239S T262A BioSTING. These mutations were also introduced to WT BioSTING in an effort to make a sensor capable of uncoupling bacterial versus eukaryotic CDNs. When cyclic dinucleotide cyclases are expressed in this mutant array, it was unexpectedly found that these mutants alone only diminished the response to cGAMP but the triple mutant Y239S, T262A, and R231A diminished but did not completely abrogate the FRET response to c-di-GMP (not shown). More work will be required to determine whether this is related to 3′3′-c-di-GMP binding before this sensor could be employed as a control for c-di-GMP secretion. In future iterations of BioSTING, unbiased approaches to make BioSTING variants with altered nucleotide specificities can be used.

BioSTING Exhibits Broad Utility for Monitoring 2′3′-cGAMP Dynamics in Live Cells

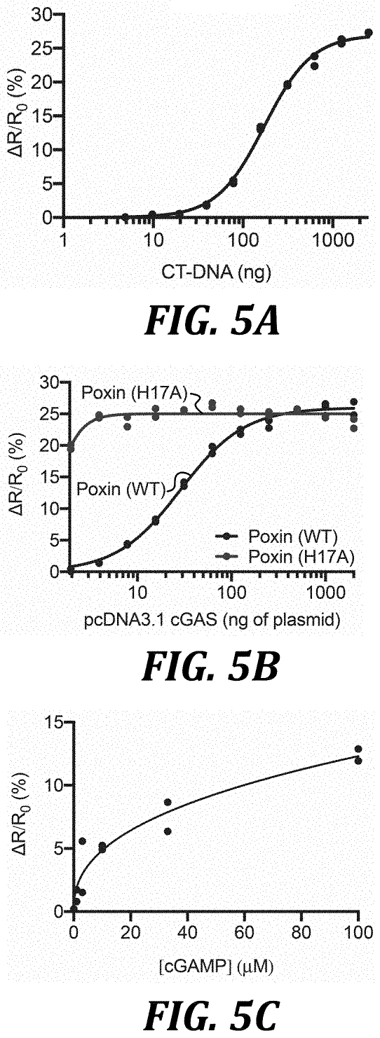

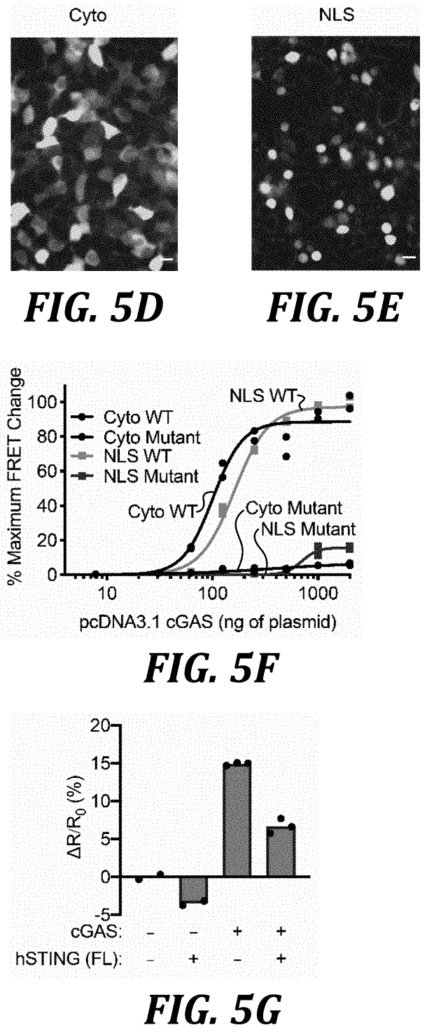

Based on the encouraging results highlighting BioSTING's ability to detect cGAMP in cells, the next step was to demonstrate BioSTING's utility to monitor modulation of cGAS activity. Previous experiments titrated pCDNA3.1-cGAS alone, resulting in simultaneous increases in both the CDN cyclase and stimulatory ligand, leading to titration curves with positive Hill coefficients. To directly measure cGAS activation cells were co-transfected with a fixed, low concentration of pCDNA3.1-cGAS with increasing amounts of calf thymus DNA (CT-DNA) ligand. As expected, at low CT-DNA levels no detectable FRET increase was observed, but as CT-DNA content was increased, an elevated FRET signal that began to saturate at the highest concentration of stimulatory ligand tested was observed (FIG. 5A). These results suggest that BioSTING is a useful tool to investigate the kinetics of cGAS activation in live cells.

The cGAS-STING pathway is immensely important for controlling viral infection and many viruses have developed methods to inhibit this pathway. Recently, vaccinia virus was reported to antagonize cGAS-STING signaling through expression of Poxin, a 2′3′-cGAMP-selective phosphodiesterase. To monitor cGAMP hydrolysis by Poxin in cells, a constant level of wild-type (WT) or catalytically dead (H17A) Poxin were expressed over a titration of cGAS plasmid. Expression of Poxin in 293T cells was confirmed by [³²P] CDN hydrolysis assays. Briefly, TLC analysis of [³²P] labeled 2′3′-cGAMP and 3′3′-c-di-AMP degradation was performed following 1 h incubation in lysates from HEK293T cells transfected with 3 μg of empty vector or plasmid expressing wildtype (WT) or catalytically-dead (H17A) Poxin. (not shown). In cells transfected with low levels of cGAS plasmid high FRET from the H17A mutant and a greatly decreased FRET response in cells expressing the WT Poxin was observed. As expected, expression of high cGAS levels overcame the capacity of Poxin to antagonize cGAMP levels (FIG. 5B). Co-expression of Poxin with DisA or WspR* had no effect on 3′3′-CDN-mediated FRET responses as compared to empty vector controls, consistent with the role of Poxin as a 2′3′-cGAMP specific hydrolase (not shown). These results demonstrate the utility of BioSTING to characterize modulators of cGAMP concentrations within living cells.

While cGAS production of cGAMP and subsequent STING activation both occur in the cytosol, recent findings have established that CDNs can be transmitted between cells through export and import mechanisms, as well as through gap junctions. Such nucleotide transfer is reported to facilitate antitumor properties and cGAMP is being explored therapeutically both alone and in combination with PD-1 blockade. The therapeutic utility of cGAMP in this context requires nucleotide import to the cytosol to promote STING inflammatory responses. To investigate the utility of BioSTING for monitoring cellular import of cGAMP, increasing concentrations of cGAMP were added to the extracellular medium. After 6 hours, cellular FRET signals in response to altered concentrations of cGAMP exhibited saturation like responses below the maximum signal associated with the sensor (FIG. 5C). The observed saturation of the response may be due to saturation of the importer operative in these cells or a consequence of the establishment of import-export equilibrium. While a thorough account of the transport mechanisms is yet to be documented, these results demonstrate that BioSTING can detect cGAMP uptake from the extracellular space and may provide a valuable tool to identify and characterize the mechanism by which cGAMP is transferred among cells.

Despite being a well-studied system, conflicting reports regarding cGAS and cGAMP localization remain. As a genetically encodable protein, it was hypothesized that BioSTING could be localized to distinct cellular compartments through the introduction of specific signal sequences. As such, a nuclear localization signal (NLS) was introduced to BioSTING, which resulted in successful localization to the nucleus (FIGS. 5D and 5E). Using a cGAS titration, cGAMP was able to be detected in both the nucleus and the cytoplasm (FIG. 5F). Consistently, the Y239S T262A BioSTING control sensor had very little change in FRET response providing additional evidence that bonafide cGAMP is being detecting in the nuclear compartment. Although these experiments only indicate that cGAMP can penetrate the nucleus, it is likely that BioSTING used in time-course and microscopy experiments will further elucidate the localization of cGAMP production under various activating conditions and can further be used to reveal mediators of nucleotide transit between cellular compartments.

Finally, BioSTING, containing the dimerization domain of full-length STING, has the potential to heterodimerize with endogenous STING. To determine the consequences of heterodimerization, full-length, human STING was expressed alone or in combination with cGAS in HEK293T cells stably expressing BioSTING. Expression of human STING in this context decreased BioSTING FRET response in the presence and absence of cGAS (FIG. 5G). Observation of a FRET decrease upon expression of STING suggests that heterodimerization leads to an altered conformation and that cells must be deficient for STING in order to attain interpretable FRET responses. Taken together, these data demonstrate that the first generation FRET biosensor, BioSTING, is highly versatile for both in vitro and cellular studies, but its application is currently limited to a STING-deficient setting.

Discussion

This example describes the development of BioSTING, a FRET-based intramolecular biosensor engineered to monitor CDNs in vitro and in cells. BioSTING maintains the native CDN binding properties of the parent protein upon which it was designed and, as such, exhibits CDN reporting capacity in physiological concentration ranges. Through a variety of in vitro and cellular studies BioSTING is established as a robust sensor of a variety of CDNs, providing real-time detection of nucleotide levels with temporal and spatial resolution.

BioSTING's ease of recombinant production, native binding properties, and simple kinetic readout make it a promising tool for investigating cyclic dinucleotides in vitro. It is shown that BioSTING can detect extracted nucleotides from cellular samples as well as enzymatic production of cyclic dinucleotides with recombinant protein. Although not investigated in this work, BioSTING can also likely be used to monitor phosphodiesterase activity for bacterial 3′3′-cyclic dinucleotides in real time. However, given the unexpectedly low dissociation rate, such application for 2′3-cGAMP hydrolysis may be limited. In addition to general enzymatic characterization of CDN synthesis, BioSTING was also utilized to characterize PF-06928215 inhibition of cGAS, demonstrating BioSTING's robust utility for characterization of small molecule modulators of CDN synthases. A key feature to be considered relates to the spectral properties of compounds under investigation. For instance, while several antimalarial compounds have been reported to inhibit cGAS activity, the intrinsic fluorescence of these compounds spectrally overlap with BioSTING and interfere with its application in this context. Accordingly, the choice of FRET partners integrated into the BioSTING platform should be rationally selected to avoid substantial spectral overlap with the compound of interest.

Despite impressive in vitro utility, the primary motivation for developing BioSTING was to create a tool capable of detecting 2′3′-cGAMP in live single cells. While the dynamic range of BioSTING is indeed narrower, the present findings support that BioSTING binds and responds to cGAMP at physiologically relevant concentrations and with sensitivity comparable to commercially available EIA based approaches. Additionally, FRET changes were successfully measured by flow cytometry, providing the first single cell measurements of CDN levels. In addition to single cell measurements, a key aspect of FRET based biosensors is the capacity to provide subcellular information about signaling dynamics. As a genetically encodable protein, localization tags can be added to target a sensor to specific cellular compartments. There is currently a debate about the localization of cGAS and therefore production of cGAMP in cells. By introducing an NLS to BioSTING the ability to restrict the sensor to the nucleus and monitor cGAMP within this compartment was demonstrated. Application of BioSTING in combination with rapid imaging microscopy is likely to provide important insight into when and where cGAMP is produced and if this differs depending upon the infectious insult, within distinct cell types, or in response to cellular damage versus pathogen encounter.

One particularly exciting application of BioSTING is for high throughput small molecule and forward genetic screening. In high throughput small molecule screening applications, BioSTING provides the ability to measure cGAMP production in live cells, which can simultaneously account for compound toxicity and cell permeability together with target engagement. In fact, Pfizer identified PF-06928215 as an inhibitor of cGAS through in vitro enzyme screening methods but the compound failed in development as a pharmaceutical due to its inability to access the cytosol. Additionally, it is anticipated that BioSTING will have utility in genetic screens to study modulation of cGAMP levels in cells. Because FRET measures can be conducted using flow cytometry, BioSTING affords the ability to conduct these studies in batch culture. Combining BioSTING flow-based sorting with disruption or overexpression libraries from mammalian and microbial pathogens will afford genome wide interrogation of CDN signaling pathways, including molecular insight into pathogen associated antagonists as well as cell intrinsic regulators of the pathway, namely cGAS activators and inhibitors and mediators of cGAMP hydrolysis, transport, and localization.

Though developed to investigate 2′3′-cGAMP cellular biology, BioSTING is also capable of investigating similar aspects of bacterial 3′3′-cyclic dinucleotides. It is demonstrated through expression of DisA and WspR* that BioSTING can detect bacterial 3′3′-CDNs in the mammalian cytosol. Thus, the potential for BioSTING to be used as a tool to investigate the timing and magnitude of bacterial CDN release in biologically relevant contexts such as during Listeria monocytogenes, Mycobacterium tuberculosis, and Chlamydia trachomatis infection, among others, is evident. In the attempts to identify CDN blind variants of BioSTING, Y239S T262A BioSTING was inadvertently identified as a c-di-GMP specific reporter. These findings suggest that further engineering of BioSTING may be possible to identify mutants sensitive to specific CDNs. Such sensors would be useful to dissect mixed CDN interactions such as during M. tuberculosis infection, in which both cGAS produced 2′3′-cGAMP and bacterial derived c-di-AMP have been implicated in STING activation. Additionally, a wide array of bacterial cyclic dinucleotides of dipurine, dipyrimidine, and mixed purine pyrimidine content were recently reported; however, only cyclic dipurine containing nucleotides were shown to robustly bind to and activate STING. It is feasible that BioSTING can be engineered through a mix of semi-randomized and rationally designed mutations to detect these more recently discovered signaling nucleotides. Finally, mutation of Tyrosine 167, which stabilizes CDN binding though a pi-stacking interaction, to an Alanine or Valine will likely generate an additional BioSTING control variant that is universally blind to all CDNs. Although outside the scope of this work, the intracellular concentration of bacterial CDNs have been reported to be similar to the Kd of BioSTING, thus it is feasible that expression of BioSTING in bacterial cells will afford interrogation of CDN dynamics within these organisms as well.

BioSTING as presently implemented is a blue, orange-based rather than far-red or luminescence-based biosensor, which limits its use to cell culture due to the limited ability for blue-orange light to penetrate tissues. Although there are many important findings to be made in cell culture, as a clinically relevant molecule, there is also an immense interest in studying 2′3′-cGAMP in vivo. A far-red FRET or luminescent version of BioSTING can be generated by replacing mKO2-mTFP fluorophores with a small circularly rotated library of a red-shifted FRET pair, BRET pair, or split luciferase and screening for increased signal upon cGAMP binding. Production of either a far-red or luminescent derivative integrated into the murine genome under either constitutive or cell specific promoters will allow for the detection of cGAMP in vivo and create a powerful model to study cGAMP activity in viral infection and autoimmune disease models.

As a STING-based biosensor, BioSTING is sensitive to heterodimerization with native STING. Thus, the sensitivity afforded by STING also leads to the limitation that BioSTING must be used in cells which either do not express or are genetically modified to lack STING. Although many important studies are tractable in this system, some investigations such as those coupling cGAMP measurements with STING-Interferon pathway activation or regulation in the same sample currently are not. To allow investigation of these exciting research areas, a version of BioSTING can be created that is incapable of heterodimerization with WT STING through engineering of the dimerization interface. This updated version will increase BioSTING's versatility while retaining the impressive sensitivity and specificity of STING.

Overall BioSTING is a powerful tool that makes many fundamental and clinically important investigations of cyclic dinucleotide biology more tractable and in some instances even feasible. In addition to the immediate application of current BioSTING versions, there is immense promise in using BioSTING as a foundation to develop a wide array of biosensors with unique CDN binding or in vivo imaging capacities. In total, BioSTING represents a versatile tool to significantly advance the current limits of knowledge related to the ever expanding and clinically relevant field of cyclic dinucleotide signaling.

Results

BioSTING Cloning

Primers for BioSTING cloning are listed in TABLE 2 and plasmids and strains are listed in TABLE 3. Prototype and GGSGG linker versions of BioSTING were generated by amplifying STING CTD with Kapa HiFi polymerase (Kapa Biosystems) using a combination of primers 1, 2, 3, and 4 from pET28b-mSTING CTD. The resulting products were ligated into pET15b-mKO2-12AA-mTFP using Spe1/Kpn1 fast digest restriction endonuclease cloning (Thermo Fisher,) and transformed into XL1-Blue chemically competent E. coli. Site directed mutagenesis was carried out by amplifying the generated pET15b-BioSTING sensor using primers 5,6 or 7,8 or 9,10, or 11,12 using Kapa HiFi polymerase. PCR purified product was DpnI digested (NEB) and transformed into XL1-Blue chemically competent E. coli. To generate pSLIK-BioSTING, pET15b-BioSTING was amplified using primers 13 and 14 for cytoplasmic expression and primers 13 and 15 to add a nuclear localization signal (NLS) from c-MYC at the C-terminus. These products there then ligated into the BsiW1 (Thermo Fisher) site of pSLIK using InFusion (Takara) then transformed into Stbl3-OneShot competent cells (Thermo Fisher).

TABLE 2
Primers used in the study.

| Primer No./ | | SEQ |
|---|---|---|
| Description | Sequence | ID NO: |
| 1) Forward | GAGGAGACTAGTTTAAATGTTGCCCACGGGCTG | 1 |
| Amplify STING | | |
| CTD no linker | | |
| 2) Reverse | GAGGAGGGTACCTTCCTGACGAATGTGCCGGAG | 2 |
| Amplify STING | | |
| CTD no linker | | |
| 3) Forward | GAGGAGACTAGTGGCGGATCCGGGGGCTTAAATGTTGCCCACGGGCTG | 3 |
| Amplify STING | | |
| CTD GGSGG | | |
| linker | | |
| 4) Reverse | GAGGAGGGTACCGCCCCCGGATCCGCCTTCCTGACGAATGTGCCGGAG | 4 |
| Amplify STING | | |
| CTD GGSGG | | |
| linker | | |
| 5) Forward | GGCATCAAGAATCGGGTTTCTTCCAACAGCGTCTACGAG | 5 |
| Y239S | | |
| QuickChange | | |
| 6) Reverse | CTCGTAGACGCTGTTGGAAGAAACCCGATTCTTGATGCC | 6 |
| Y239S | | |
| QuickChange | | |
| 7) Forward | CTGTATCCTGGAGTACGCCGCCCCCTTGCAGACCC | 7 |
| T262A | | |
| QuickChange | | |
| 8) Reverse | GGGTCTGCAAGGGGGCGGCGTACTCCAGGATACAG | 8 |
| T262A | | |
| QuickChange | | |
| 9) Forward | CCCAGCAAAACATCGACCATGCTGGCATCAAGAATCGG | 9 |
| R231H | | |
| QuickChange | | |
| 10) Reverse | CCGATTCTTGATGCCAGCATGGTCGATGTTTTGCTGGG | 10 |
| R231H | | |
| QuickChange | | |
| 11) Forward | CCCCAGCAAAACATCGACGCTGCTGGCATCAAGAATCGG | 11 |
| R231H | | |
| QuickChange | | |
| 12) Reverse | CCGATTCTTGATGCCAGCAGCGTCGATGTTTTGCTGGGG | 12 |
| R231A | | |
| QuickChange | | |
| 13) Forward | TGATCACTAGCGTACGACCATGGGCAGCAGCCATCATCATC | 13 |
| Amplify | | |
| BioSTING for | | |
| pSLIK | | |
| 14) Reverse | TCTTCCAATTCGTACGTCATCCGCCAAAACAGCCAAG | 14 |
| Amplify | | |
| BioSTING for | | |
| pSLIK | | |
| 15) Reverse | TCTTCCAATTCGTACGTCAGTCCAACTTGACCCTCTTGGCAGCAGGTC | 15 |
| Amplify | CGCCAAAACAGCCAAGC | |
| BioSTING for | | |
| pSLIK with | | |
| NLS tag | | |

TABLE 3
Primers used in the study.

| Name | Use |
|---|---|
| pET15b-mKO2-12AAmTFP | Contained FRET fluorophores |
| pET15b-BioSTING | Bacterial expression vector |
| pET15b-BioSTING (Y239S | Bacterial expression vector |
| T262A) | |
| pSLIK-Empty Vector | Eukaryotic expression vector |
| pSLIK-BioSTING | Eukaryotic expression vector |
| pSLIK-BioSTING | Eukaryotic expression vector |
| pSLIK-BioSTING (R231A) | Eukaryotic expression vector |
| pSLIK-BioSTING (R231H) | Eukaryotic expression vector |
| pSLIK-BioSTING (Y239S | Eukaryotic expression vector |
| T262A) | |
| pSLIK-BioSTING (R231A | Eukaryotic expression vector |
| Y239S T262A) | |
| pSLIK-BioSTING (R231H | Eukaryotic expression vector |
| Y239S T262A) | |
| pSLIK-NLS BioSTING | Expresses nuclear localized |
| | BioSTING |
| pSLIK-NLS BioSTING | Expresses nuclear localized |
| (Y239S T262A) | BioSTING (Y239S T262A) |
| pcDNA3-empty vector | Vector Control in Eukaryotic cells |
| pcDNA3-cGAS | Expresses full-length human cGAS |
| | in Eukaryotic cells |
| pcDNA4-DisA | Expresses DisA in Eukaryotic cells |
| pcDNA4-WspR* | Expresses WspR* in Eukaryotic cells |
| pcDNA4-Poxin (WT) | Expresses Poxin (WT) in Eukaryotic cells |
| pcDNA4-Poxin (H17A) | Expresses Poxin (H17A) in Eukaryotic |
| | cells |
| pcDNA3-hSTING (FL) | Expresses hSTING in Eukaryotic cells |
| pSPEEDET-mRECON | Bacterial expression vector |
| pet28-mSTING-CTD | Bacterial expression vector |
| pet20b-DisA | Bacterial expression vector |
| pET28a-His6-SUMOmcGAS | Bacterial expression vector |

Protein Expression and Purification

Recombinant 6×-His tagged SUMO-mcGAS, B. subtilis (B.s.) DisA, mSTING-CTD, and mRECON were expressed and purified as summarized below (see also Luteijn, R. D. et al. SLC19A1 transports immunoreactive cyclic dinucleotides. Nature 573, 434-438 (2019), incorporated herein by reference in its entirety). Briefly, plasmids for mcGAS, DisA, mSTING-CTD, and mRECON expression were transformed into Rosetta (DE3)pLysS chemically competent cells. Overnight cultures of the resulting transformed bacteria were inoculated into 1.5 L of LB broth at a 1:100 dilution. Bacterial cultures were grown to OD₆₀₀ 0.4-0.6 at 37° C. after which protein expression was induced by the addition of 0.5 mM isopropyl β-D-1-thiogalactopyranoside (IPTG) for 20 hours at 16° C. Bacteria were harvested by centrifugation, and the cell pellets were resuspended in Buffer A [50 mM Tris-Cl pH=8.0, 300 mM NaCl, 20 mM Imidazole, 5 mM β-Mercaptoethanol (BME), and 1 mM phenylmethylsulfonyl fluoride (PMSF)]. Cells were lysed by sonication and clarified lysate was bound to HisPur NiNTA Resin (Thermo Scientific). The resin was washed with 100-200 column volumes of buffer A and bound proteins were eluted in Buffer B [50 mM Tris-Cl pH=7.4, 300 mM NaCl, 300 mM Imidazole, 5 mM β-Mercaptoethanol (BME), and 1 mM phenylmethylsulfonyl fluoride (PMSF)]. Following NiNTA chromatography, His6-SUMO-mcGAS was exchanged into Buffer C [20 mM Tris-Cl pH 7.4, 250 mM NaCl, 1 mM dithiothreitol (DTT)] and further purified by Heparin Sepharose chromatography. Bound cGAS was eluted over 250 mM to 1000 mM NaCl gradient. The resulting purified proteins were analyzed by SDS-PAGE, exchanged into storage buffer [40 mM Tris pH 7.5, 100 mM NaCl, 20 mM MgCl₂] using PD-10 desalting columns (GE Healthcare), snap frozen, and stored at −80° C. until use.

For BioSTING expression, plasmids encoding BioSTING variants were transformed into Rosetta (DE3)pLysS chemically competent cells. Overnight cultures of the resulting transformed bacteria were inoculated into 1 L of LB broth and grown as above. At an OD₆₀₀ of 0.5-0.7, protein expression was induced by the addition of 0.2 mM isopropyl β-D-1-thiogalactopyranoside (IPTG) for 6 hours at 18° C. Bacteria were harvested by centrifugation, and the cell pellets were resuspended in Buffer D [50 mM Tris-Cl pH=7.5, 100 mM NaCl, 20 mM Imidazole, 5 mM β-Mercaptoethanol (BME), and 1 mM phenylmethylsulfonyl fluoride (PMSF)]. Cells were lysed by sonication and clarified lysate was bound to HisPur NiNTA Resin (Thermo Scientific, Waltham, Mass.). The resin was washed with 100-200 column volumes of Buffer D and bound proteins were eluted in Buffer E [50 mM Tris-Cl pH=7.5, 100 mM NaCl, 300 mM Imidazole, 5 mM β-Mercaptoethanol (BME), and 1 mM phenylmethylsulfonyl fluoride (PMSF)]. The resulting proteins were concentrated and further purified by gel filtration on a Superdex 200 column (GE Healthcare) using storage buffer [40 mM Tris pH 7.5, 100 mM NaCl, 30 mM MgCl₂, supplemented with 0.5 mM TCEP]. Protein samples were tested for purity by SDS-PAGE followed by Coomassie Brilliant Blue staining. Fractions with high purity were pooled, concentrated, flash frozen, and stored at −80° C. until use in biochemical assays.

Synthesis of [³²P] 2′3′-Cyclic GMP-AMP and [³²P] 3′3′-Cyclic Di-AMP

[³²P] Radiolabeled cyclic dinucleotides (CDNs) were synthesized enzymatically using α-[³²P] ATP (Perkin-Elmer) and recombinant SUMO-mcGAS (2′3′-cGAMP) or B.s. DisA (3′3′-c-di-AMP) and affinity purified using mSTING-CTD and mRECON, as follows:

[³²P] cGAMP was synthesized enzymatically by incubating 0.33 μM U-[³²P] ATP (Perkin-Elmer) with 250 μM unlabeled GTP, 1 μg of Interferon Stimulatory DNA 100mer, and 1 μM of recombinant His-tagged cGAS in binding buffer [40 mM Tris pH 7.5, 100 mM NaCl, 20 mM MgCl₂] at 37° C. overnight. Subsequently, recombinant cGAS was removed from the reaction mixture by incubation with HisPur Ni-NTA resin (Thermo Scientific) for 30 min. The sample was transferred to a minispin column (Thermo Scientific) to elute the crude [³²P] cGAMP sample. The resulting [³²P] cGAMP was purified further using recombinant mSTING-CTD. 100 μM mSTING-CTD was bound to HisPur Ni-NTA resin for 30 minutes on ice. The resin was washed two times to remove unbound mSTING-CTD. The resulting resin was incubated with the remaining crude cGAMP synthesis reaction mixture for 30 min on ice. Following removal of the supernatant, the Ni-NTA resin was washed five times with ice cold binding buffer. The resin was then incubated with 100 μL of binding buffer for 10 min at 95° C. and transferred to a minispin column to elute [³²P] cGAMP.

[³²P] c-di-AMP was synthesized as follows: briefly, 1 μM α-[³²P] ATP (Perkin-Elmer) was incubated with 1 μM of recombinant DisA in binding buffer at 37° C. overnight. The reaction mixture was boiled for 5 min at 95° C. and DisA was removed by incubation with HisPur Ni-NTA resin. The sample was transferred to a minispin column (Thermo Scientific) to elute the crude [³²P] c-di-AMP sample. The resulting [³²P] c-di-AMP was further purified using recombinant His-tagged mRECON. 100 μM His-tagged mRECON was bound to HisPur Ni-NTA resin for 30 min on ice. The resin was washed two times to remove unbound RECON. The resulting resin was incubated with the remaining crude [³²P] c-di-AMP sample for 30 minutes on ice. Following removal of the supernatant, the Ni-NTA resin was washed five times with ice cold binding buffer and then incubated with 100 μL of binding buffer for 5 min at 95° C. The slurry was then transferred to a minispin column to elute [³²P] c-di-AMP.

Affinity purified CDNs were analyzed by Thin Layer Chromatography (TLC) on Polygram CEL300 PEI TLC plates (Machery-Nagel) in buffer containing 1:1.5 (vol/vol) saturated (NH₄)₂SO₄ and 1.5 M NaH₂PO₄ pH 3.6. [³²P] radiolabeled CDNs were visualized by exposure onto PhosphorImager screens, which were developed using a Typhoon FLA 9000 biomolecular imager (GE Healthcare) and determined to be ˜99% pure.

Nucleotide Binding Assays

[³²P] Radiolabeled cyclic dinucleotide binding assays were performed using DRaCALA (Roelofs, K. G., et al. Differential radial capillary action of ligand assay for high-throughput detection of protein-metabolite interactions. Proceedings of the National Academy of Sciences 108, 15528-15533 (2011), incorporated herein by reference in its entirety). Binding assays were performed in binding buffer [40 mM Tris pH 7.5, 100 mM NaCl, 20 mM MgCl₂] at room temperature. To determine binding affinities, two-fold serial dilutions of proteins were incubated with ˜1 nM of [³²P] radiolabeled CDNs for at least 10 minutes. To determine binding specificities, proteins were pre-incubated with 500 μM excess, unlabeled nucleotides for 10 minutes, followed by incubation with ˜1 nM of [³²P] radiolabeled CDNs for at least 10 minutes. Samples were then blotted onto nitrocellulose membranes and allowed to air dry. [³²P] radioactivity was visualized by exposure onto PhosphorImager screens, which were developed using a Typhoon FLA 9000 biomolecular imager (GE Healthcare). Non-radioactive 2′3′-cGAMP (Invivogen), 3′3′-cGAMP (Invivogen), 3′3′-c-di-AMP (Invivogen, San Diego, Calif.), and 3′3′-c-di-GMP (BIOLOG Life Science Institute, Bremen, Germany) were purchased and diluted in endotoxin free water.

In Vitro FRET Assays

5-10 μM purified BioSTING proteins were incubated with increasing concentrations of cyclic dinucleotides within a black flat bottom opaque 96-well plate (Greiner Bio-One) in activity buffer [40 mM Tris pH 7.5, 100 mM NaCl, 20 mM MgCl₂]. mTFP and FRET fluorescence was monitored using a fluorimeter (BioTek Synergy H1 Hybrid Reader, BioTek Instruments) with the following parameters (unless otherwise stated): 458 nm excitation, 490 nm emission for mTFP and 600 nm emission for mKO2. Assay parameters were calculated using Prism Software. Kd and EC50 were determined using nonlinear fit and all other parameters were calculated using linear fit, according to literature precedent (Surdo, N. C., et al. FRET biosensor uncovers cAMP nano-domains at β-adrenergic targets that dictate precise tuning of cardiac contractility. Nature Communications 8, 15031 (2017), incorporated herein by reference in its entirety).

Enzyme Activity Assays

For DisA enzyme activity assays, 5-10 μM purified BioSTING was incubated with increasing concentrations of recombinant B.s. DisA within a black 96-well plate in activity buffer [40 mM Tris pH 7.5, 100 mM NaCl, 20 mM MgCl₂]. Enzyme assays were initiated by the addition of 1 mM ATP, and the enzyme reactions were allowed to proceed for 2 hours at 37° C.

For cGAS enzyme activity assays, 5-10 μM purified BioSTING was incubated with increasing concentrations of recombinant SUMO-mcGAS within a black 96-well plate in activity buffer [40 mM Tris pH 7.5, 100 mM NaCl, 20 mM MgCl₂]. Enzyme assays were initiated by the addition of 1 mM ATP, 1 mM GTP and 1 μg ISD (FIGS. 2B, 7A, and 7B) or by the addition of 250 μM ATP, 250 μM GTP, and 50 ng ISD (FIGS. 2C, 2D, and 7C). Enzyme assays were allowed to proceed for 2 hours at 37° C. cGAS inhibitor (PF-06928215) was purchased from Sigma-Aldrich and diluted in sterile DMSO.

For all assays, FRET activity was monitored as above.

2′3′-cGAMP Extraction

HEK293T cells were plated at a density of 750,000 cells per well of a 6-well cell culture plate. The next day cells were transfected with the indicated amounts of pcDNA3.1-hcGAS vector using PEI transfection reagent (Polysciences). 24 hours later the cells were harvested by centrifugation and washed once with ice-cold PBS. Cell pellets were resuspended in ice-cold 80% Optima, HPLC grade methanol (Fisher Scientific) and incubated on ice for 20 minutes. Cells were further lysed by sonication. Following centrifugation, cellular extracts were completely dried under vacuum and stored at −20° C. until use. For FRET assays, extracts were resuspended in activity buffer [40 mM Tris pH 7.5, 100 mM NaCl, 20 mM MgCl₂] containing 5-10 μM purified BioSTING and transferred to a black 96-well plate. FRET activity was monitored using a fluorimeter as described above. Quantification requires a standard curve of known cGAMP concentrations in the sample buffer done at the time of analysis.

Cell Lines

Human Embryonic Kidney (HEK) 293T cells were grown in Dulbecco's Modified Eagle Medium (DMEM) (Gibco) supplemented with 10% (v/v) heat-inactivated FBS (HyClone), 1 mM sodium pyruvate, 2 mM L-Glutamine (Thermo Fisher), 100 U mL⁻¹ penicillin, 100 μg mL⁻¹ streptomycin and maintained at 37° C. in 5% CO2 in a humidified incubator.

Lentivirus Production and Transduction

VSV-G pseudotyped, self-inactivating lentivirus was prepared by transfecting a semi-confluent 10 cm dish of HEK293T cells with 4 μg of psPAX2, 2 μg of pCMV-VSV-G, together with 4 μg of pSLIK lentiviral vector using Poly(ethyleneimine) (PEI). Growth medium was replaced 24 hours after transfection and cell culture supernatants were collected at 48 and 72 hours after transfection and filtered through a 0.45 μm filter.

For lentiviral transduction, HEK293T cells were seeded at a density of 2 to 4 million cells per 10 cm dish. The following day, cells were transduced with 5 mL of filtered lentiviral supernatant. 24 hours later the cell culture medium was removed and replaced with standard cell culture medium supplemented with 2 μg per mL puromycin (Gibco). For all subsequent experiments, lentivirus-transduced cells were passaged and maintained in selection medium containing puromycin.

Intracellular FRET Assays

For experiments using CDN cyclases, HEK293T cells stably expressing the indicated BioSTING constructs under a doxycycline-inducible promoter were plated at a density of 750,000 cells per well of a 6-well cell culture plate. The next day, the cells were transfected with the indicated amounts of cyclase-encoding plasmids using PEI transfection reagent. One hour later, biosensor expression was induced by the addition of Doxycycline Hydrochloride (1 μg mL⁻¹) (Sigma-Aldrich). 24 hours later the cells were harvested by centrifugation and resuspended in ice-cold PBS. Biosensor activity was determined by FACS analysis. To contextualize results, a negative control in which no cGAMP is present and a positive control where cGAMP is abundant should be run in each assay to quantify the lower and upper bounds of FRET activation.

For electroporation experiments, HEK293T cells stably expressing wild-type BioSTING were plated at a density of five million cells per 10 cm dish in cell culture medium supplemented with Doxycycline (1 μg mL⁻¹) to induce biosensor expression. The next day cells were harvested by trypsinization and electroporated with the indicated concentrations of 2′3′-cGAMP using SF Cell Line 4D-Nucleofector X Kit according to the manufacturer's protocols (Lonza). Following electroporation, the cells were resuspended in ice-cold PBS and analyzed by FACS analysis.

For extracellular 2′3′-cGAMP stimulations, HEK293T cells stably expressing wild-type BioSTING were plated at a density 750,000 cells per well of a 6-well cell culture plate in cell culture medium supplemented with Doxycycline (1 μg mL⁻¹) to induce biosensor expression. The next day, the indicated concentrations of 2′3′-cGAMP were added to the culture medium. 6 hours later the cells were harvested by centrifugation and resuspended in ice-cold PBS. Biosensor activation was determined by FACS analysis.

Flow Cytometry

To prepare cells for flow cytometry, cell culture media was aspirated, and the cells were harvested in ice-cold PBS. The resuspended cells were then analyzed using a LSR II flow cytometer (BD) with the following voltages: FSC-A/H/W-350, SSC-A/H/W-240, BV510(mTFP)-360, PE(mKO2)-380, and BV570(FRET)-375 volts. Data was then analyzed using FlowJo software (Tree Star).

2′3′-cGAMP Enzyme Immunoassay (EIA)

HEK293T cells were plated at a density of 750,000 cells per well of a 6-well cell culture plate. The next day cells were transfected with the indicated amounts of pcDNA3.1-hcGAS vector using PEI transfection reagent. 24 hours later the cells were harvested by centrifugation and washed once with ice-cold PBS. Cell lysates were prepared using the 2′3′-cGAMP EIA protocol and 2′3′-cGAMP was quantified according to the manufacturer's instructions (Arbor Assays).

Western Blotting

HEK293T cells were plated at a density of 750,000 cells per well of a 6-well cell culture plate. The next day cells were transfected with 1 μg pcDNA3.1-hcGAS or empty vector using PEI transfection reagent. 24 hours later cells were harvested by centrifugation, and the cell pellets were lysed in Pierce RIPA buffer (Thermo Scientific) supplemented with Halt Protease and Phosphatase Inhibitor Cocktail (Thermo Scientific). Lysates were clarified by centrifugation, and protein content was normalized using Pierce BCA Protein Assay Kit (Thermo Scientific). In total, 30 μg of protein per condition were loaded onto Any kD Mini-PROTEAN TGX Precast Protein Gels (Bio-Rad) and separated by SDS-PAGE. Proteins were then transferred onto nitrocellulose membranes (Bio-Rad) at 100V for 90 minutes at 4° C. The membranes were then air dried for one hour and blocked in 5% Blotto, non-fat milk (Santa Cruz Biotechnology) dissolved in 1×TBS for one hour. Membranes were probed overnight in 5% Bovine Serum Albumin (Fisher Scientific) dissolved in 1×TBS-T with anti-cGAS Rabbit mAb (1:1000) and anti-β-Actin Mouse mAb (1:1000) (Cell Signaling Technology). Proteins were visualized using IRDye 800CW Goat anti-Rabbit IgG Secondary Antibody (1:10000) and IRDye 680RD Goat anti-Mouse IgG Secondary Antibody (1:10000) (LI-COR Biosciences). All wash steps were carried out using 1×TBS-T. Blots were imaged using an Odyssey Fc System (LI-COR Biosciences). Rabbit anti-cGAS (D1D3G) and mouse anti-R-Actin (8H10D10) monoclonal antibodies were obtained from Cell Signaling Technology.

### Example 2

This Example describes the development of another embodiment of the fusion construct for detecting CDNs, described above. Specifically, this Example describes the development of a Förster resonance energy transfer (FRET)-based biosensor, referred to CDA5, which is based on the Listeria monocytogenes c-di-AMP effector protein Lmo0553 and can provide real-time detection of 3′3′-cyclic di-adenosine monophosphate (c-di-AMP) dynamics in vitro and in vivo.

Introduction

Bacterial growth, reproduction, and survival demand rapid, accurate, and coordinated responses to internal and environmental cues. These responses are commonly relayed by nucleotide second messengers which dynamically change concentration through rapid synthesis and degradation. The nucleotide second messenger 3′3′-cyclic di-adenosine monophosphate (c-di-AMP) is unique in that it is essential in diverse bacterial genera and regulates clinically and industrially relevant processes such as osmotic stress responses, cell wall metabolism, and metabolic homeostasis. C-di-AMP is known to be produced by five classes of di-adenylate cyclases, although most bacteria contain only one of two main cyclases: the membrane associated CdaA and DNA binding DisA. Bacillus subtilis, the model organism used in this study, interestingly encodes cdaA, disA, and a spore restricted cyclase, cdaS. Due to redundancy, it is possible to delete individual cyclases in B. subtilis without dramatic phenotypic consequences. C-di-AMP is degraded by 4 classes of phosphodiesterase although most bacteria only encode one or two of these enzymes. Bacillus subtilis encodes both gdpP and pgpH, which again, due to redundancy, allows for deletion of single phosphodiesterases without significant physiological defects.

Despite being a second messenger of growing interest, only a small handful of studies have explored the internal and external signals which regulate the activity of c-di-AMP cyclases and phosphodiesterases. Multi-hour exposure to glutamine, potassium, and light have been reported to impact signal levels but the mechanisms by which nucleotide levels change have either not been defined or have been linked to altered phosphodiesterase expression. Additionally, investigations of protein-protein interactions influencing intracellular c-di-AMP levels have been performed elucidating DacA regulation by the cistronic GlmM and YbbR proteins. It is believed that the only studies which clearly identified post-translational c-di-AMP responses to environmental conditions have been: first, that a wide array of bacteria when non-growing but energized quickly accumulate more c-di-AMP in low osmotic conditions relative to high osmotic conditions, and second, that (p)ppGpp, which accumulates during amino acid starvation, inhibits c-di-AMP phosphodiesterases. Such foundational studies remain largely elusive because detection methods are underdeveloped.

C-di-AMP is currently quantified using either mass spectrometry or an enzyme immunoassay which are both relatively low throughput and expensive methods providing only a snapshot of the population average at a single time point. Additionally, an RNA-aptamer based biosensor has also been developed to detect c-di-AMP. Unfortunately, this biosensor has not been able to be utilized because it has a long k_{off} which obscures c-di-AMP decreases, suffers from stochasticity in fluorescent ligand uptake, and lacks an internal control. To partially remedy this dearth, a CDA-Luc assay was recently developed, which is an inexpensive and higher throughput method for quantifying c-di-AMP. Although this method is likely to be invaluable to many investigations, it is still limited to destructive snapshots of the population average.

Thus, an intramolecular FRET biosensor for c-di-AMP is an ideal tool to complement existing techniques as it would allow for nondestructive and rapid resampling of c-di-AMP in single cells. Intramolecular FRET biosensors have been central to diverse investigations of nucleotide second messenger regulation in both eukaryotic and bacterial cells. A FRET-based biosensor for c-di-AMP can be used to interrogate important phenomena, which are currently intractable using current c-di-AMP detection methods including: sub-population responses to environmental conditions, mother/daughter cell heterogeneity, and regulation during infection.

Intramolecular FRET biosensors are fusion proteins that combine compatible fluorophores with a native binding protein for the ligand of interest. The locations of these fluorophores are engineered such that ligand binding induces a conformational change which moves the donor fluorophore either closer to or further from the acceptor fluorophore generating altered energy transfer. This shift in energy transfer causes a change in the fluorescent signal quantified as the ratio of: energy transferred to and released by the acceptor fluorophore divided by energy released by the donor fluorophore. The FRET ratio reports on the binding state of the biosensor allowing for back calculation of the free ligand concentration within the solution or cell. Thus, FRET biosensors are powerful, entirely genetically encodable, internally controlled, and, due to their native effector protein scaffold, have physiologically relevant binding parameters.

This Example presents the design and characterization of CDA5: a FRET biosensor designed around the Listeria monocytogenes c-di-AMP binding protein, Lmo0553 (Sureka, K. et al. The cyclic dinucleotide c-di-AMP is an allosteric regulator of metabolic enzyme function. Cell 158, 1389-1401 (2014), incorporated herein by reference in its entirety). It is demonstrated that CDA5 retains relevant native binding characteristics, produces a robust FRET response upon the addition of c-di-AMP, and successfully reports on the concentration of c-di-AMP in individual bacterial cells. Furthermore, it is reported that B. subtilis grown in diluted LB media rapidly decreases its intracellular c-di-AMP concentration and that c-di-AMP phosphodiesterase and cyclase mutants have respectively higher and lower corresponding FRET signals. Additionally, it is reported that, in all conditions tested and unlike c-di-GMP, c-di-AMP concentrations in single cells follow a unimodal distribution. These investigations not only identify a new facet of c-di-AMP biology but also establish CDA5 as a versatile platform which will facilitate a wealth of basic and applied investigations of the essential bacterial signaling molecule c-di-AMP.

Results

Overall Structure of Lmo0553 in Complex with Cyclic-Di-AMP

Lmo0553 was previously identified as a Listeria monocytogenes protein of unknown function that binds c-di-AMP at physiologically relevant concentrations. Despite numerous attempts to investigate the function of Lmo0553, its physiology has remained elusive. Thus, the investigation was directed to determine Lmo0553's basis of recognition and molecular response to c-di-AMP. The crystal structure of Lmo0553 in complex with c-di-AMP was determined at 1.6 Å resolution, as well as the structure of free Lmo0553 at 2.48 Å resolution. The atomic models have good agreement with the crystallographic data and the expected bond lengths, bond angles, and other geometric parameters (TABLE 4).

TABLE 4
Summary of Crystallographic Information. Values in
parentheses refer to the highest resolution shell.

| | Lmo0553-c-di-AMP | | | |
|---|---|---|---|---|
| | complex | | Free Lmo0553 | |
| Space Group | P3₁21 | | P2₁ | |
| Cell dimensions | | | | |
| a, b, c (Å) | 87.8, 87.8, 107.2 | | 37.2, 70.6, 88.7 | |
| α, β, γ (°) | 90, 90, 120 | | 90, 99.8, 90 | |
| Resolution¹ | 50-1.60 | (1.66-1.60) | 50-2.48 | (2.57-2.48) |
| Rmerge (%) | 5.5 | (43.4) | 9.7 | (37.3) |
| I/σI | 32.9 | (5.2) | 10.3 | (2.6) |
| Redundancy | 8.1 | (8.1) | 2.9 | (2.7) |
| Completeness | 100 | (100) | 97 | (97) |
| R_{work} (%) | 16.7 | (19.8) | 24.1 | (31.5) |
| R_{free} (%) | 20.2 | (24.7) | 28.1 | (38.6) |
| Average B-factors | | | | |

| | | |
|---|---|---|
| Protein | 21.3 | 46.5 |
| Ligand/ion | 15.7 | — |
| Water | 28.7 | — |
| R.m.s deviation bond | 0.014 | 0.011 |
| lengths (Å) | | |
| R.m.s deviation bond | 1.6 | 1.5 |
| angles (°) | | |
| PDB Accession Code | | |

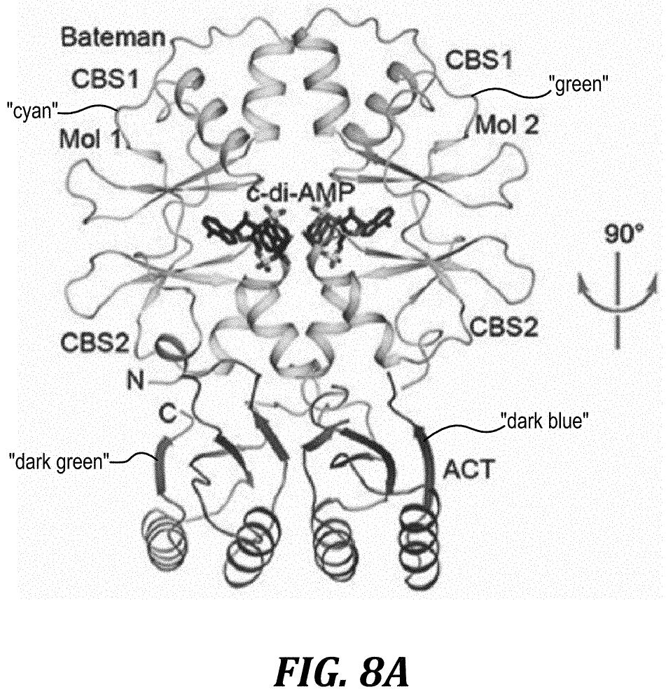

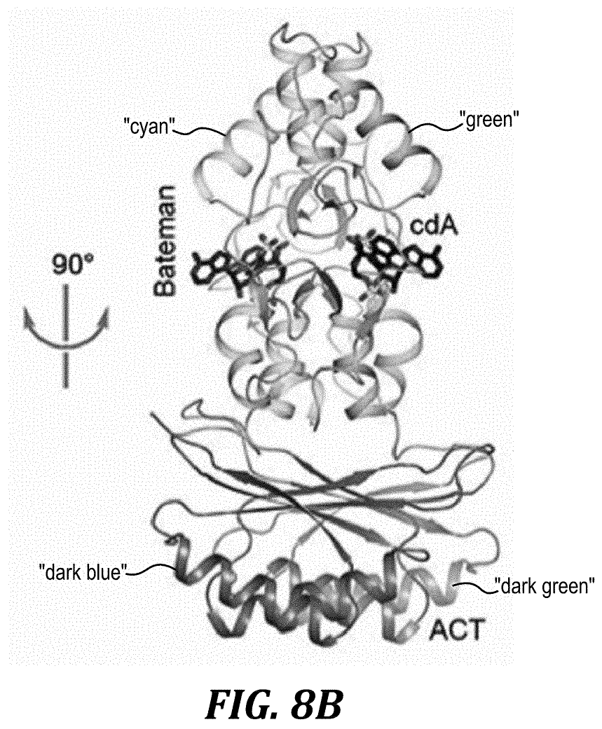

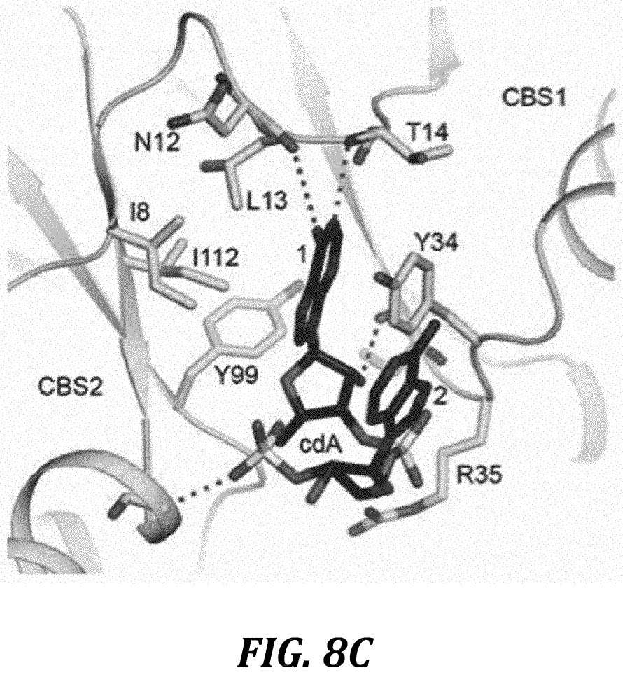

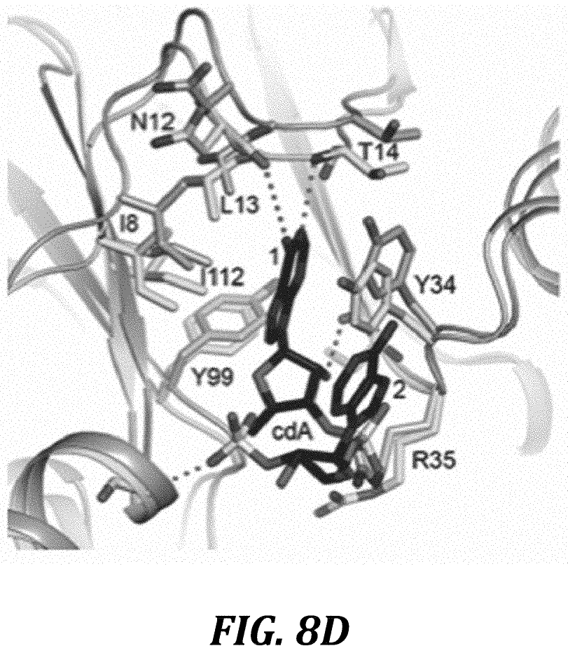

Lmo0553 contains tandem CBS motifs (Bateman domain) followed by an ACT domain. A dimer of Lmo0553 was observed in the crystals in which the Bateman domains of the two monomers contact each other in a head-to-head fashion, forming a disc-like dimer (FIGS. 8A and 8B). This dimer is similar to other structures of CBS motif-containing proteins. As predicted, the C-terminal ACT domain of Lmo0553 adopts a ferredoxin fold (βαββαβ). This domain also forms a dimer, producing a structure consisting of an 8-stranded β-sheet packed against four α-helices on one face. The Bateman domain dimer sits atop the β-sheet of the ACT dimer, forming an extensive interface. The Bateman and ACT domains are diagonally swapped in the dimer, such that the Bateman domain of monomer 1 packs against the ACT domain of monomer 2 (FIG. 8A).

Two c-di-AMP molecules are bound in the central cavity of the Bateman domain dimer, one on each face of the disc (FIG. 8B). The dinucleotide adopts a folded conformation, with both bases in the anti configuration. The adenine of the first nucleotide (labeled 1 in FIG. 8C) is buried between the CBS1 and CBS2 motifs of a monomer. This adenine makes extensive interactions with the protein and is recognized specifically, with a hydrogen-bond to its N1 and N6 atoms. Tyr34 is p-stacked against one face of the adenine ring, while several hydrophobic residues flank the other face. In contrast, the adenine of the second nucleotide (labeled 2 in FIG. 8C) makes few direct interactions with the protein. The most notable interaction of the second nucleotide is the 5-prime phosphate which interacts with the side chain of Arg35. In the free as compared to c-di-AMP bound Lmo0553 structure, the side chain of Arg35 assumes a different conformation and occupies the binding site of c-di-AMP (FIG. 8D). In the c-di-AMP complex structure, residues that contact the adenine base of the first nucleotide show substantial conformational differences as well. These changes in the binding site are likely the trigger for the overall changes in the organization of the dimer upon c-di-AMP binding.

Large Conformational Changes Upon c-Di-AMP Binding Guide Biosensor Design

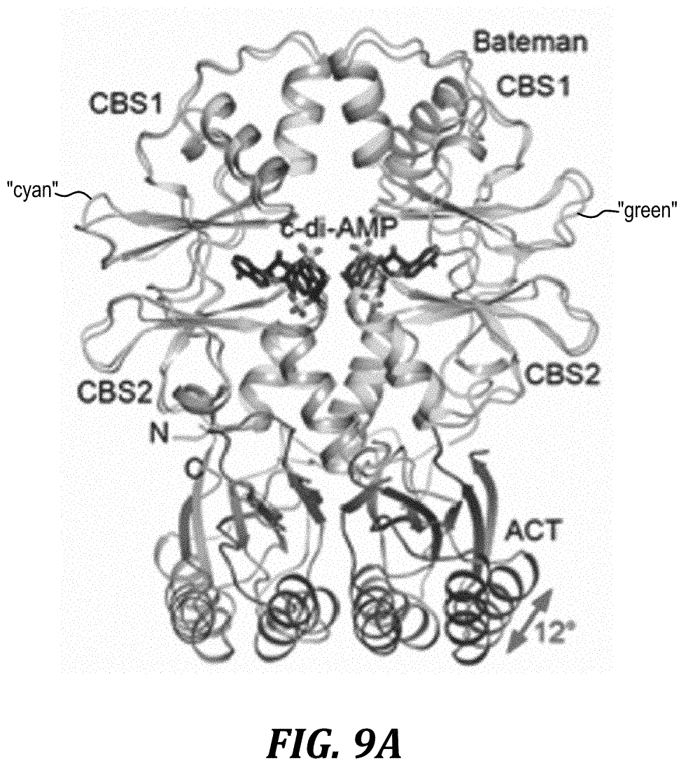

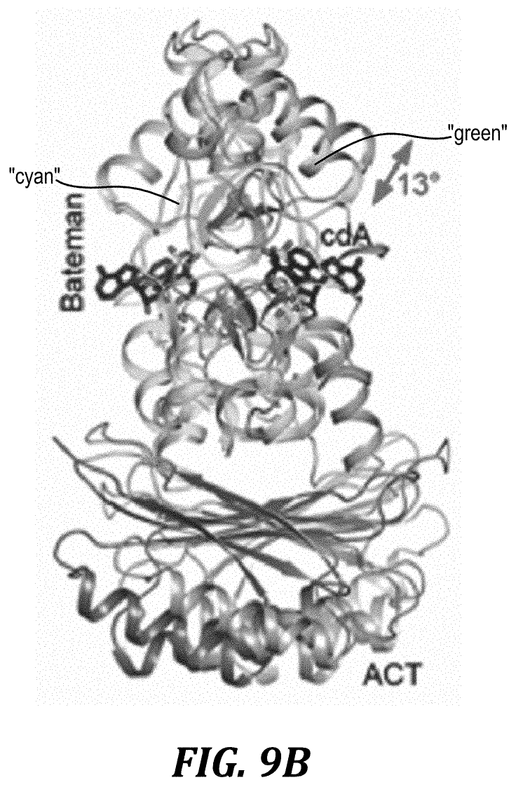

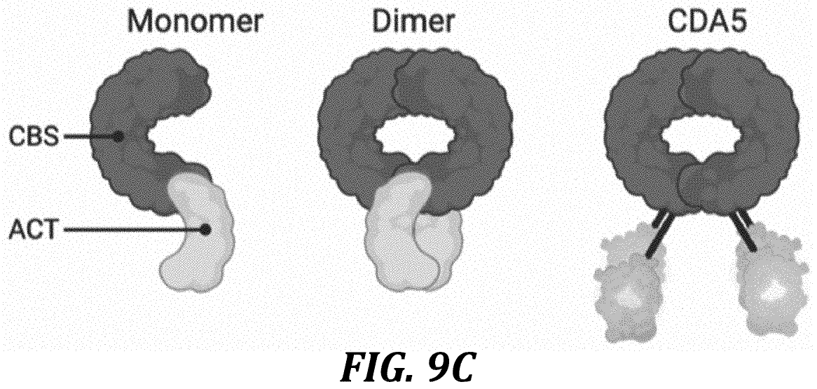

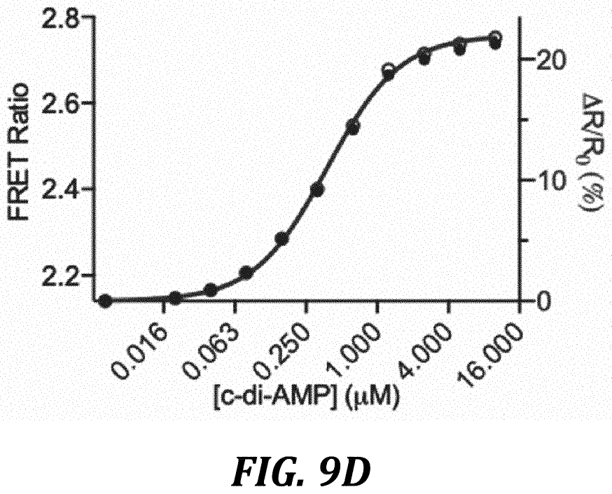

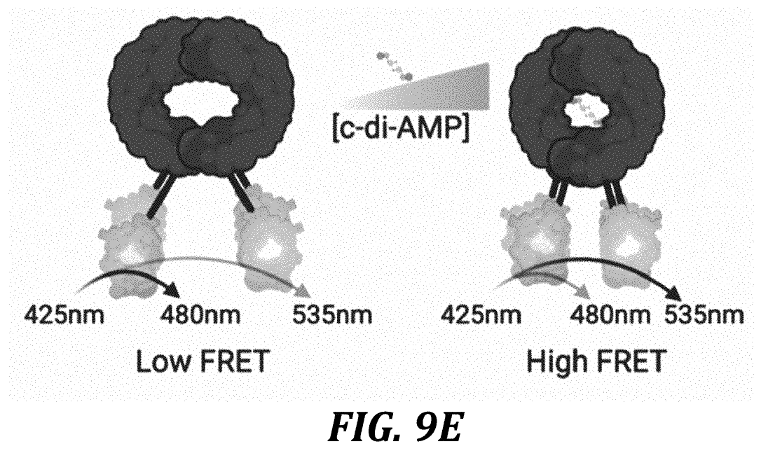

Although elucidating the native physiology of Lmo0553 remains a subject of investigation, a detailed understanding of the molecular consequences of c-di-AMP binding led a hypothesis that Lmo0553 could serve as a scaffold for development of a Förster resonance energy transfer (FRET) biosensor. FRET is exquisitely sensitive to angstrom level changes, therefore the structure of free Lmo0553 was compared to that of the c-di-AMP complex to identify conformational differences (FIGS. 9A and 9B). After the Bateman domain of one monomer is overlaid, the orientation of the Bateman domain in the other monomer differs by 130 (FIG. 2B). In addition, the orientation of the ACT domain differs by 12° (FIG. 2A).

To leverage these structural rearrangements, the FRET pair eCFP and eYFP were respectively fused to the N and C termini of full length Lmo0553. This construct was purified and found to be stable but no FRET response was observed upon the addition of c-di-AMP suggesting that fluorophores were too distant or oriented such that structural rearrangements were not detected. Additional engineering may have allowed for the generation of a FRET biosensor utilizing full length Lmo0553, but without a response to optimize, these efforts would have been arduous and potentially unsuccessful.

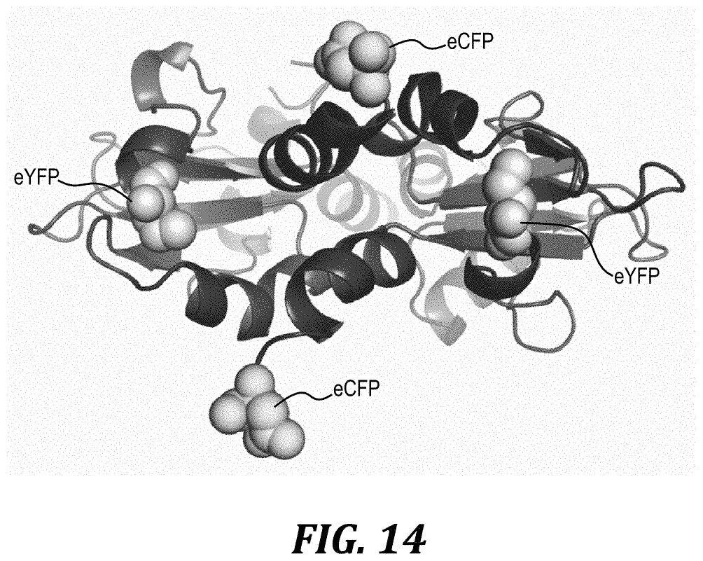

Reanalyzing the crystal structures, it was noted that truncation of the ACT domain might lead to a better biosensor scaffold. The N-terminal Bateman domain of Lmo0553 is distinct from the ACT domain, encompasses the entire binding domain, undergoes large structural changes upon c-di-AMP binding, and, if isolated, would bring fluorophores into close contact increasing the potential to detect FRET ratio changes (FIG. 14). To generate this second iteration biosensor, Lmo0553 was truncated at N-127 and fused to eCFP and eYFP (FIG. 9C) to best leverage expected large conformational shifts and avoid disruption of the globular domain.

This second iteration c-di-AMP biosensor was recombinantly produced and found to be stable in solution. Excitingly, increasing concentrations of c-di-AMP caused a robust, 23% FRET increase with an EC50 of 0.38 μM (FIG. 9D). Thus, when this biosensor binds c-di-AMP, the chromophores of the flanking fluorophores come in closer contact allowing for increased energy transfer producing an elevated FRET response (FIG. 9E). Satisfied by the magnitude of FRET response, this biosensor was named “CDA5” (cyclic-di-AMP biosensor based on Lmo0553) and sought to characterize it biochemically.

Affinity and Specificity of CDA5

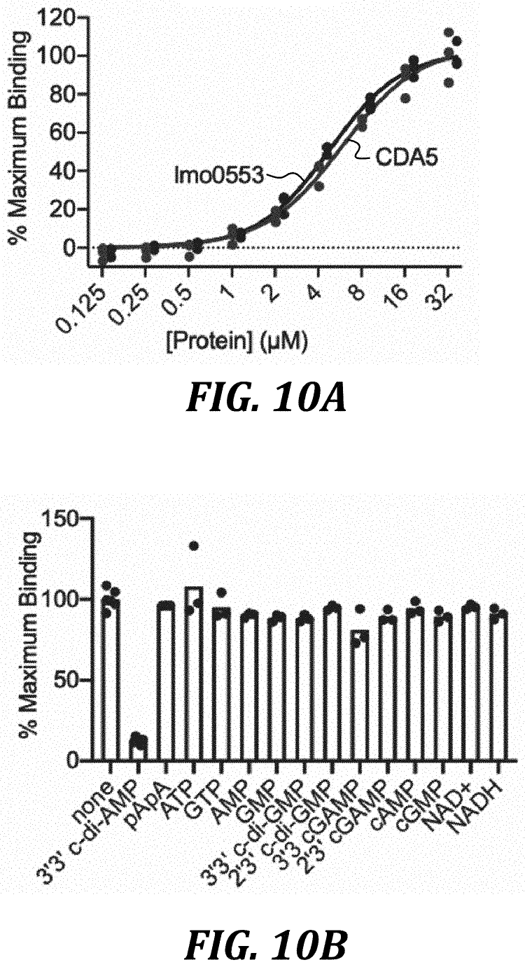

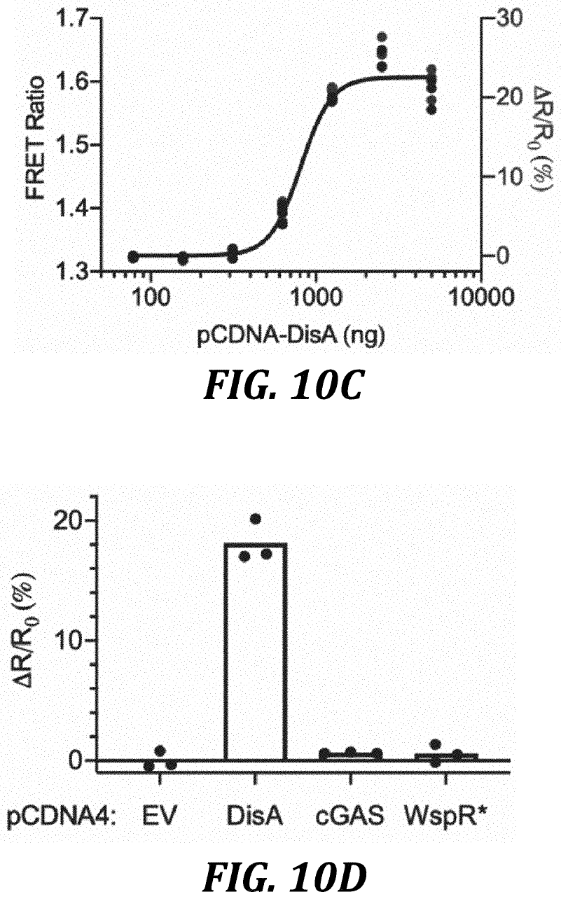

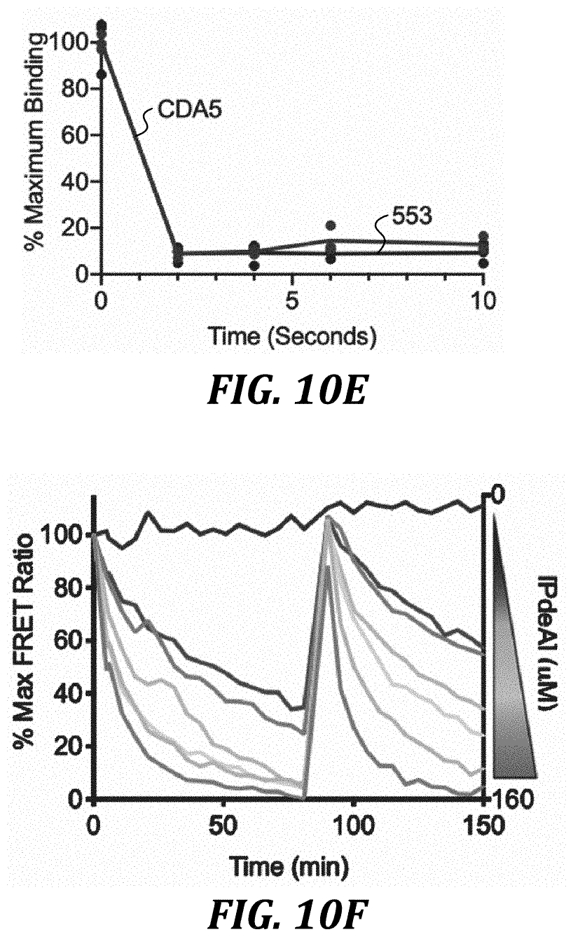

Although initial FRET response assays suggested that CDA5 retains physiologically relevant biochemical parameters, it was sought to further validate this assumption by comparing it directly to full length Lmo0553. DRaCALA analysis (Roelofs, K. G., et al. Differential radial capillary action of ligand assay for high-throughput detection of protein-metabolite interactions. Proc. Natl. Acad. Sci. U.S.A. 108, 15528-15533 (2011), incorporated herein by reference in its entirety) using [³²P]-labeled c-di-AMP was employed which revealed an apparent Kd of 4.83 μM and 5.87 μM for Lmo0553 and CDA5 respectively (FIG. 10A). This result demonstrates unaltered affinity for c-di-AMP supporting the hypothesis that neither truncation nor fusion with eCFP or eYFP altered ligand binding thermodynamics. To ensure that CDA5 engineering did not alter the previously reported specificity of Lmo0553, the DRaCALA assay was again employed. This was done by competing bound radiolabeled c-di-AMP with excess unlabeled nucleotides. CDA5 and Lmo0553 both demonstrated exquisite specificity for c-di-AMP, as only unlabeled c-di-AMP but no other nucleotide in a wide array of monophosphate, triphosphate, cyclic, and dicyclic purine containing nucleotides could compete off radioactive c-di-AMP (FIGS. 10B and 14). These data support the hypothesis that CDA5 engineering did not alter critical biochemical parameters.

To further interrogate CDA5 specificity, a complex cytosolic environment was utilized where an array of cyclic dinucleotide (CDN) cyclases could be reliably ectopically expressed. Specifically, as described in Example 1, a biosensor was engineered that was capable of broadly detecting CDNs, particularly 2′3′-cGAMP, in the eukaryotic cytosol, and this system was adapted for CDA5. It was observed a titratable FRET response to the c-di-AMP cyclase DisA and, as expected, no response to high levels of either WspR*, a 3′3′-c-di-GMP cyclase, or cGAS, a 2′3′-cGAMP cyclase, further validating the specificity of CDA5 for c-di-AMP (FIGS. 10C and 10D).

CDA5 Reversibility

To be a reliable reporter, CDA5 must rapidly respond to both increasing and decreasing concentrations of c-di-AMP. CDA5 relies on a native effector protein scaffold that is hypothesized to rapidly respond to nucleotide second messenger fluctuations in order to carry out post-translational responses. It was found that FRET responses were stable immediately upon addition of c-di-AMP providing evidence of a kₒₙ less than the sampling limit of 10 seconds. Similarly, the k_{off} rate was also unable to be quantitated due to sampling limitations. Utilizing the DRaCALA specificity assay, excess unlabeled c-di-AMP was added to both CDA5 and Lmo0553 pre-bound with [³²P]-labeled c-di-AMP and immediately sampled for binding. Unlabeled c-di-AMP competed off bound [³²P]-labeled c-di-AMP completely within two seconds providing evidence of a k_{off} less than this time interval (FIG. 10D). Despite not being able to quantify kₒₙ and kof rates with these assays, these results suggest that CDA5 can rapidly report on c-di-AMP fluctuations.

To further investigate the ability of CDA5 to repetitively detect c-di-AMP increases and decreases, similar to what was hypothesize is occurring in bacterial cells, c-di-AMP was cycled in solution by increasing the concentration using a bolus of c-di-AMP and decreasing the concentration using the c-di-AMP phosphodiesterase, PdeA. As expected, it was observed that: PdeA caused a FRET decrease proportional to enzyme concentration, addition of a bolus of c-di-AMP restored the FRET response, and finally, the FRET response decreased again proportional to the concentration of PdeA (FIG. 10E). This assay provides evidence that CDA5 is responsive to dynamic c-di-AMP fluctuations and also that CDA5 is a useful platform for kinetic investigations in vitro.

Development of a c-di-AMP Blind Control

Characterization of CDA5 indicated that it can reliably quantitate c-di-AMP in simplified systems but, recognizing that complexities can occur in native systems, it was sought to develop a point mutant control version of CDA5 that does not bind c-di-AMP. Such a control would provide the capacity to separate FRET signal due to bonafide changes in c-di-AMP from other phenomena such as fluorophore quenching and protein-protein interactions.

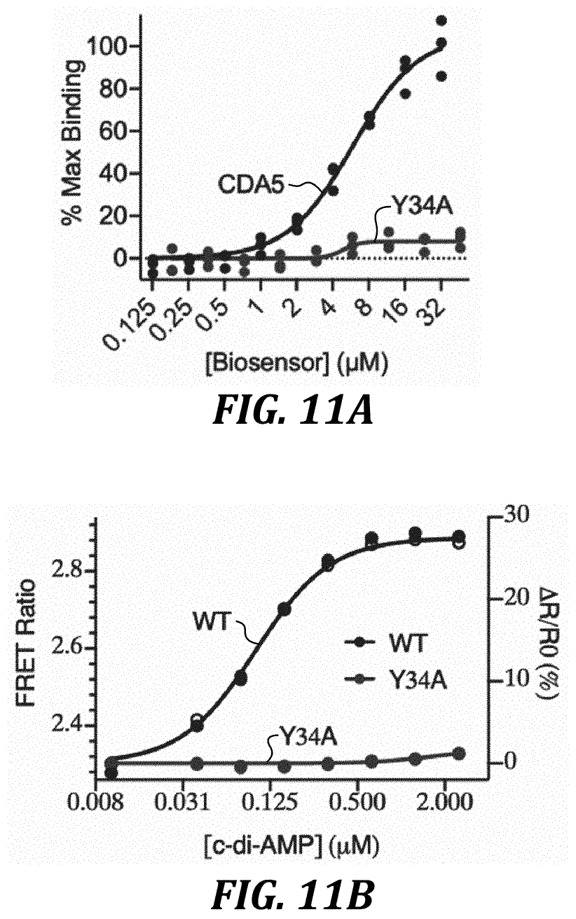

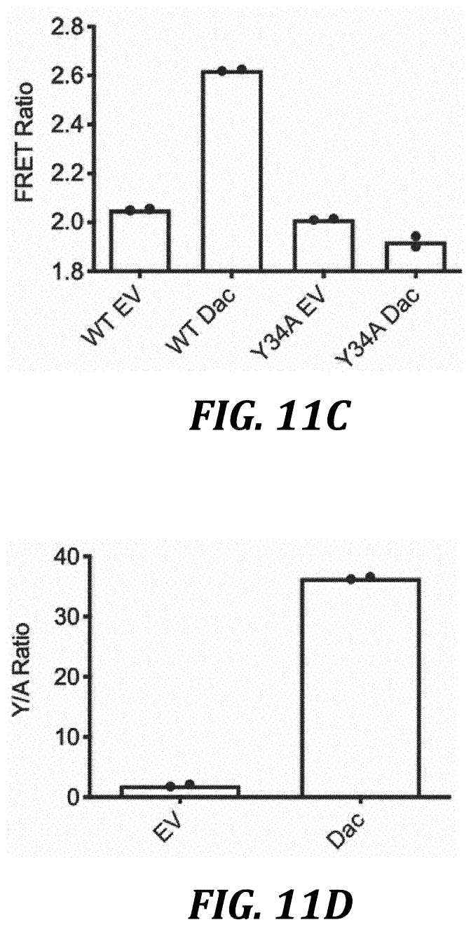

Analysis of the crystal structure of Lmo0553 identified tyrosine-34 coordination of c-di-AMP binding via p-stacking (FIG. 8C). It was hypothesized that by replacing the stabilizing tyrosine ring with an alanine (Y34A), c-di-AMP binding would be minimized in a manner unlikely to disrupt protein stability. Recombinant Y34A CDA5 was found to be stable in solution and analyzed for binding using the DRaCALA binding assay. As hypothesized, c-di-AMP bound to CDA5 but not the Y34A CDA5 point mutant control (FIG. 11A). Next, increasing concentrations of c-di-AMP were added to purified CDA5 and Y34A CDA5 and analyzed by plate reader assay for FRET response. CDA5 but not Y34A CDA5 produced a robust FRET increase upon the addition of c-di-AMP (FIG. 11B). Together these results suggest that Y34A CDA5 remains unbound and in the apo conformation even in the presence of c-di-AMP.

The next step was to validate Y34A CDA5 in Escherichia coli, which is a complex model bacterial organism that does not naturally produce c-di-AMP but can be made to ectopically express a c-di-AMP cyclase and synthesize c-di-AMP. Thus, E. coli was transformed with a plasmid to express WT CDA5 or Y34A CDA5 as well as a second plasmid encoding the soluble domain of the c-di-AMP cyclase, DacA or an empty vector and then analyzed for FRET response by flow cytometry (not shown). E. coli carrying WT CDA5 produced a robust FRET response while Y34A CDA5 had an, albeit minor, FRET decrease (FIG. 11C). It was hypothesized that the minor FRET response is due to altered levels of non-specific protein-protein interactions. Regardless, these results reinforce the utility of a nonbinding control such as Y34A CDA5 control to provide confidence that observed FRET responses are due to changes in ligand concentration and not other phenomena. It is often useful to also calculate the ratio of WT CDA5 to Y34A CDA5 FRET responses into a ‘Y/A Ratio’ (FIG. 11D). This metric, both combines the control biosensor data and declutters data making results more clear.

CDA5 Detects Unimodal Bacillus subtilis Responses to Media Alteration

CDA5 was then expressed in Bacillus subtilis to study native c-di-AMP regulation. B. subtilis is a model organism encoding a large array of c-di-AMP cyclases and phosphodiesterases but not a homologue to Lmo0553, which could cause disruptive heterodimerization. It was found that rich media like LB is necessary to attain robust CDA5 expression but, at the same time, it is also important to use media with low autofluorescence to clearly quantitate FRET ratios. Due to the necessity of rich media to express CDA5, back dilution into minimal media was not ideal due to the large metabolism changes required to transition to fully biosynthetic growth. It was found that one could back dilute B. subtilis expressing CDA5 into 10% LB media, which retains a complex nutrient content and also has low enough autofluorescence to clearly quantitate FRET by flow cytometry.

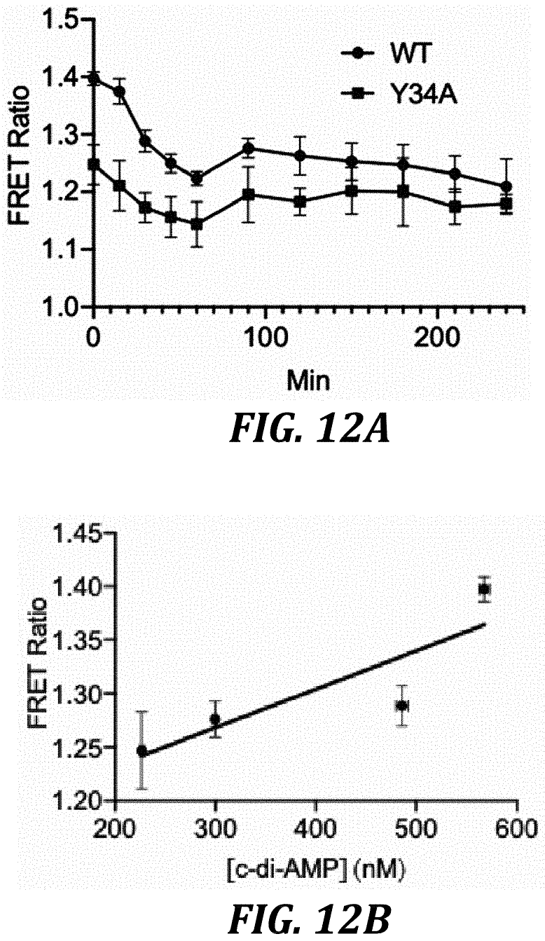

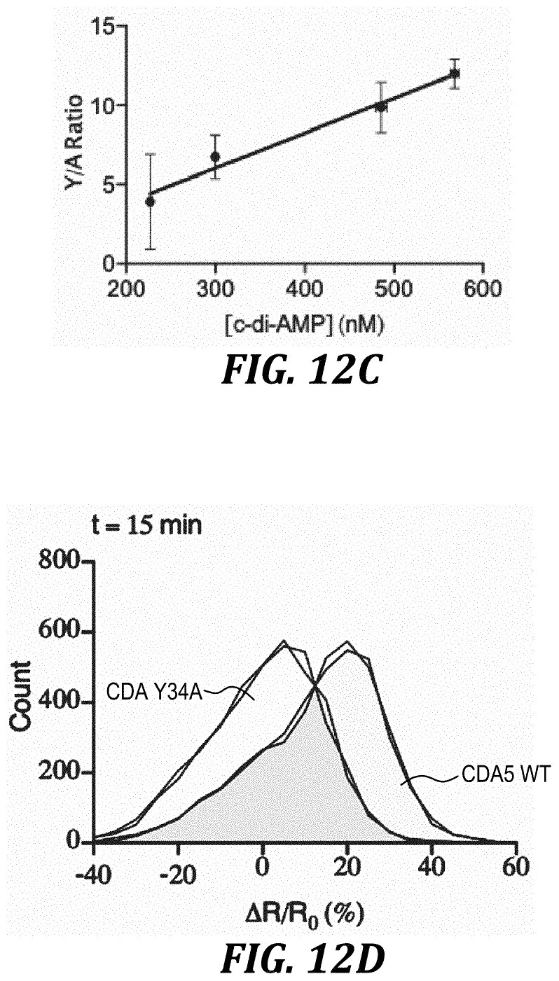

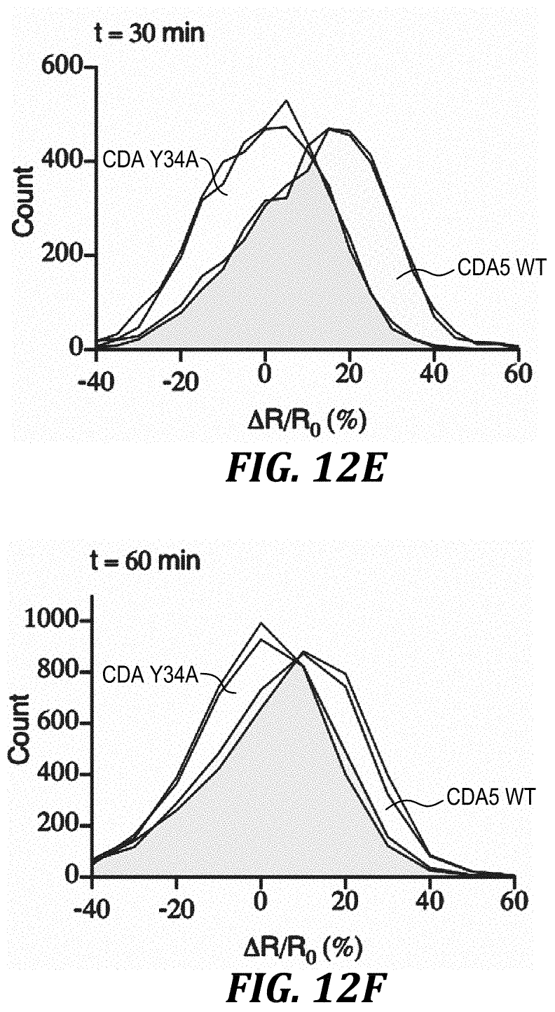

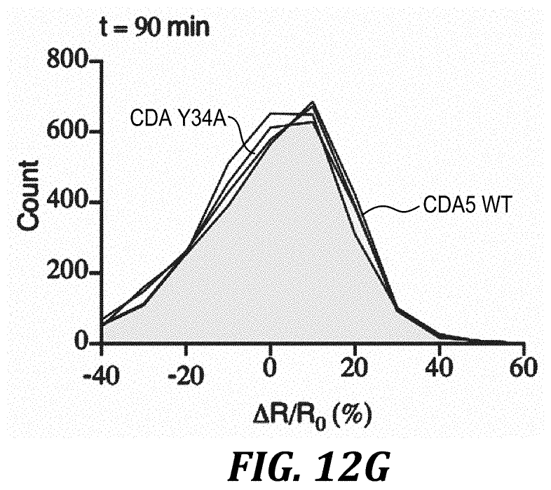

In this diluted LB media, FRET changes were tracked overtime and a steady FRET decrease was observed during the entire 180 minutes of growth until the WT and Y34A sensor had nearly the same FRET ratio (FIG. 12A). To verify this unexpected result, the sample was split to simultaneously detect c-di-AMP using the flow-based FRET assay and the gold standard for c-di-AMP quantification, mass-spectrometry. These results were plotted on XY plots graphing c-di-AMP measured by mass spectrometry versus either the WT CDA5 FRET ratio (FIG. 12B) or the Y/A ratio combining WT CDA5 and Y34A CDA5 ratios (FIG. 12C). Although both the WT CDA5 FRET ratio and the Y/A ratio both correlated with c-di-AMP as measured by mass spec, the Y/A ratio highly correlated with c-di-AMP highlighting the importance of both the Y34A CDA5 control and the Y/A metric.

Flow cytometry allows for the collection of an enormous amount of informative single cell data providing an excellent opportunity to understand more about c-di-AMP dynamics. To understand more about the mechanics of this population level c-di-AMP decrease, WT CDA5 and Y34A CDA5 single cell data was normalized by the control biosensor's population average and plotted as histograms (FIGS. 12D-12G). This analysis revealed a progressive unimodal population shift as the WT biosensor more closely overlays Y34A CDA5 over time, tracking with population level results. This data also suggests that the vast majority, if not all, of the B. subtilis cells are responding in a coordinated fashion to readjust to media conditions.

CDA5 Detects Unimodal c-Di-AMP Differences Between Bacillus subtilis Mutants

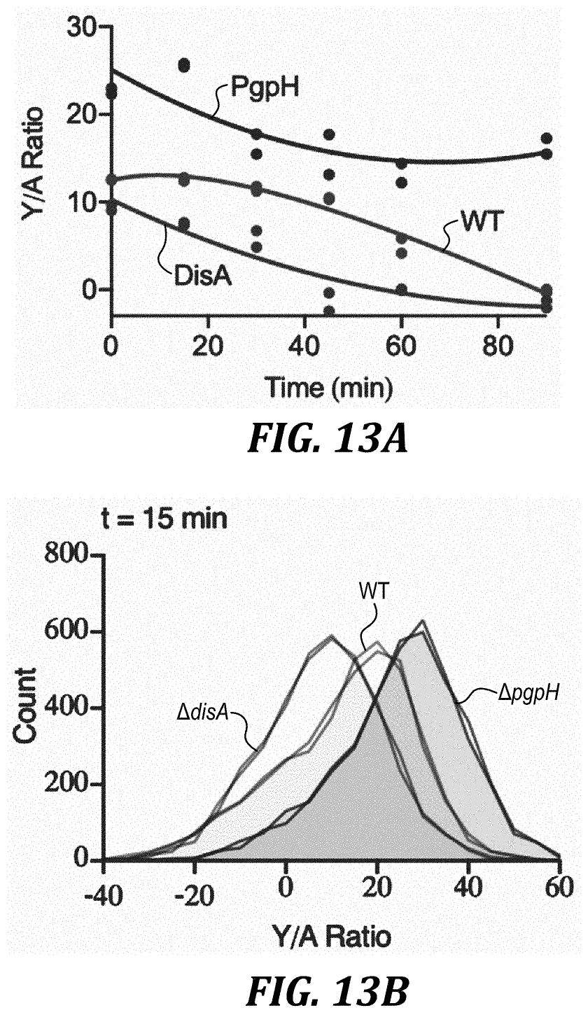

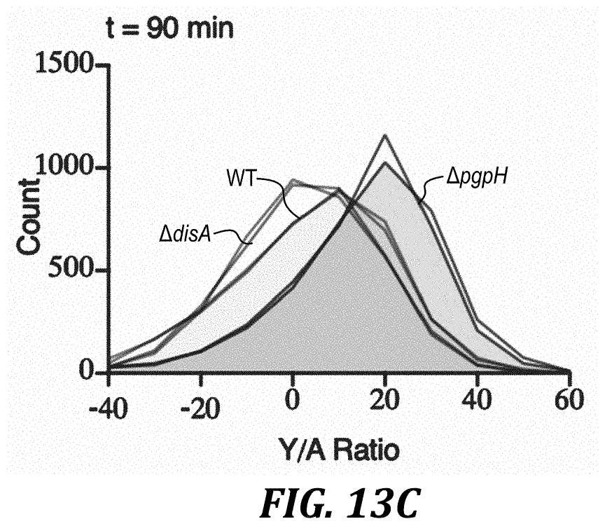

To better contextualize the biologic meaning of the FRET ratios detected in WT B. subtilis, the next step to detect c-di-AMP differences between mutants defective in either a c-di-AMP cyclase or phosphodiesterase. ΔpgpH, a mutant lacking the phosphodiesterase PgpH, had higher Y/A ratios due to elevated c-di-AMP; while a mutant lacking the cyclase DisA, ΔdisA, had lower Y/A ratios due to reduced c-di-AMP production (FIG. 13A). All three strains experienced different degrees of FRET ratio decreases. ΔdisA decreased most quickly, followed by WT, and ΔpgpH decreased minimally and remained elevated throughout the experiment. At the single cell level Y/A ratios for all strains again decreased in a unimodal fashion following the population average (FIGS. 13B-13C). More experiments will be required to elucidate the mechanism by which ΔpgpH but not other strains retain elevated c-di-AMP in reduced LB media. Importantly, this data provides convincing evidence that CDA5 detects physiologically relevant differences in c-di-AMP in B. subtilis is a useful tool to investigate bacterial c-di-AMP dynamics.

Discussion

In this Example, the development of CDA5, a FRET based biosensor capable of detecting the essential signaling molecule c-di-AMP within individual bacterial cells, is described. Through rational design based on the Lmo0553 crystal structure, a stable biosensor was generated that retains relevant native binding characteristics as well as a nucleotide blind control which improves measurement accuracy. In vivo, CDA5 allowed for the detection of c-di-AMP differences over time and between mutants of B. subtilis. Interestingly, analysis at the single cell level revealed unimodal c-di-AMP shifts providing evidence that the entire bacterial population responds in a coordinated fashion. This is particularly notable because it is in contrast to the bacterial nucleotide second messenger c-di-GMP, which is regulated in a largely bimodal manner.

In addition to its ability to monitor native c-di-AMP regulation, CDA5 is also capable of facilitating diverse investigations of c-di-AMP in other contexts. CDA5 is easy to produce, specific, and provides a kinetic readout making it a good platform to study c-di-AMP enzymology. For example, CDA5 can be used to detect protein-protein or small molecule dependent activation or inhibition of c-di-AMP cyclases and phosphodiesterases in vitro. Such work may allow for the development of new antibiotics targeting this essential signaling molecule. Some interactions, especially those of membrane associated proteins, are difficult to model with recombinant protein and are better investigated in an ectopic cytosolic environment. CDA5 expressed in such a model system would facilitate these investigations. For example, CDA5 expressed in E. coli would provide a platform for the interrogation of important protein-protein interactions controlling c-di-AMP synthesis and degradation. Additionally, c-di-AMP is known to be detected by but not produced by eukaryotic cells during infection and perhaps also by certain bacteria like Pseudomonas aeruginosa in multicellular environments. CDA5 expressed by these organisms may be able to detect accumulated c-di-AMP accelerating these interesting investigations.

Although alternative uses are promising, the primary motivation for the development of the CDA5 biosensor was to more thoroughly and efficiently investigate native c-di-AMP regulation. Current tools, including the recently developed CDA-Luc assay, are well suited for detecting c-di-AMP at the population level, but the ability to measure c-di-AMP kinetically or at the single cell level is lacking. Thus, CDA5 is a major advance which allows for a wealth of new investigations.

A major finding using the c-di-GMP biosensor was mother-daughter cell heterogeneity, which allows for different roles within a population. Similarly, it is hypothesized that there will be replication phase dependent c-di-AMP fluctuations in bacteria that naturally produce c-di-AMP. However, rather than mother-daughter cell heterogeneity, it is hypothesized that the intracellular concentration of c-di-AMP is linked to periods of rapid peptidoglycan synthesis during bacterial elongation due to the close link between c-di-AMP and cell wall homeostasis. Such insights could lead to greater understanding of morphology and virulence differences between c-di-AMP mutants.

Furthermore, the c-di-GMP biosensor was recently utilized to detect regulation of c-di-GMP signaling during macrophage infection. Due to the avirulence of mutants which hyper- or hypo-produce c-di-AMP, it is hypothesized that similar, if not more profound, regulation is occurring during infection of eukaryotic cells. Such investigations will help elucidate the role of c-di-AMP during infection for a multitude of clinically important organisms including Streptococcus pneumoniae, Listeria monocytogenes, and Mycobacteria tuberculosis.

CDA5, similarly to previous c-di-GMP biosensor studies, will also facilitate the identification of diverse environmental stimuli that regulate c-di-AMP at the population and subpopulation level. In this study, a unimodal c-di-AMP response to specific environmental conditions was detected, but CDA5 can elucidate a more thorough understanding of c-di-AMP regulation via use of an arrayed media library containing a wide variety of nutrients and stress conditions. In addition to advancing basic biology, such studies may also facilitate the development of clinical interventions which alter intracellular c-di-AMP concentrations.

CDA5 is highly functional in its current form but the platform can be optimized to improve utility. One such improvement is the use and subsequent optimization of alternative fluorophores. For example, a far-red shifted FRET pair could be utilized allowing for simultaneous non-overlapping fluorescence with BFP-tagged proteins. Similarly, a BRET pair could be developed for c-di-AMP detection within mammalian tissue or biofilms. Another type of improvement is modification of the nucleotide binding pocket to increase affinity or alter specificity. For example, it may be possible to engineer additional hydrogen bonding interactions to increase the affinity for c-di-AMP allowing for detection of lower concentrations of nucleotides. Such a sensor would be able to detect low levels of c-di-AMP found within a mammalian cytosol during infection or within a non-c-di-AMP producing bacteria in a biofilm environment. Similarly, the binding pocket of CDA5 may be able to be modified to detect the growing class of CBASS cyclic di-nucleotides produced as part of the anti-bacteriophage response. A final type of improvement would be modification of the dimerization domain. It may be possible to reverse polar interactions in the dimerization domain to avoid potentially obfuscating interactions with full length Lmo0553. This would mainly be useful for investigations in Listeria monocytogenes or Enterococcus faecalis which respectively encode lmo0553 or a homologue. Alternatively, unless Lmo0553 is the protein of interest, such studies could also be done in a lmo0553 null background as no phenotype has been identified for this protein to date.

CDA5 is a powerful tool which makes a large class of investigations now feasible. In addition to current capabilities, there are clear next steps to modify CDA5 and apply it to even more diverse studies. Thus, CDA5 holds exceptional promise for accelerating the understanding of the essential bacterial signaling molecule, c-di-AMP.

Methods

Cloning

Primers for CDA5 cloning are listed in TABLE 5, plasmids in TABLE 6, and strains in TABLE 7. CDA5 was generated by subcloning lmo0553 from the previously generated pET28a-lmo0553 vector (Sureka, K. et al. The cyclic dinucleotide c-di-AMP is an allosteric regulator of metabolic enzyme function. Cell 158, 1389-1401 (2014), incorporated herein by reference in its entirety) using Kapa HiFi polymerase (Kapa Biosystems) using primers 1 and 2. The resulting product was ligated into pET15b-eCFP-12AA-eYFP using Spe1/Kpn1 fast digest restriction endonuclease cloning (Thermo Fisher) and transformed into XL1-Blue chemically competent E. coli. To generate pSLIK-CDA5, pET15b-CDA5 was amplified using primers 3 and 4 and added to the BsiW1 (Thermo Fisher) site of pSLIK using InFusion (Takara) then transformed into Stbl3-OneShot competent cells (Thermo Fisher). pET15b-CDA5-Y34A was made using site directed mutagenesis by amplifying the generated pET15b-CDA5 construct using primers 5 and 6 using Kapa HiFi polymerase. PCR product was purified (Promega) and digested using DpnI (NEB) and then transformed into XL1-Blue chemically competent E. coli. To generate pHT264-Bs-CDA5, a gBlock codon optimized for Bacillus subtilis expression (IDT) was purchased and amplified using primers 7 and 8. The resulting product was ligated into pET15b using Not1 fast digest restriction endonuclease cloning (Thermo Fisher) and transformed into XL1-Blue chemically competent E. coli generating pET15b-Bs-CDA5. pET15b-Bs-CDA5-Y34A was generated as above using primers 9 and 10. Finally, pHT264-Bs-CDA5 and pHT264-Bs-CDA5-Y34A were generated by amplifying pET15b-Bs vectors with primers 11 and 12. The resulting product was ligated into pHT264 using BamH1/Smal fast digest restriction endonuclease cloning (Thermo Fisher) and transformed into XL1-Blue chemically competent E. coli.

TABLE 5
Primers.

| Primer | | | SEQ | |
|---|---|---|---|---|
| No. | Name | Sequence | ID NO: | Description |
| 1 | lmo0553 | GAGGAGactagtatgTTGATAAAAAACCTTT | 16 | Amplify the CDA5 |
| | CBS F | GTATCCCTAAAATAAATTTAACAAC | | Lmo0553 scaffold |
| 2 | lmo0553 | GAGGAGggtaccATTCCAGCCATCTTGGAGC | 17 | |
| | CBS R | AAAC | | |
| 3 | CDA5 | tgatcactagcgtacgaccATGGTTAGCAAG | 18 | Amplify CDA5 for |
| | pSLIK F | GGAGAGGAGC | | lentiviral expression |
| 4 | CDA5 | tcttccaattcgtacgTTACTTATAAAGCTC | 19 | |
| | pSLIK R | ATCCATTCCATGTGTAATTCC | | |
| 5 | CDA5 | GGCTATCCATTTACTTGAAGAATCTGGTGCT | 20 | Quickchange to make |
| | Y34A F | CGCTGTGTTCCTGTATTAGACG | | Y34A |
| 6 | CDA5 | CGTCTAATACAGGAACACAGCGAGCACCAGA | 21 | |
| | Y34A R | TTCTTCAAGTAAATGGATAGCC | | |
| 7 | Bs CDA5 | GAGGAGggcggccgcATGGTAAGTAAAGGAG | 22 | Amplify Bs-CDA5 |
| | gBlock F | AGGAGTTGTTC | | gBlock for subcloning |
| 8 | Bs CDA5 | GAGGAGgcggccgccTTTATACAGTTCGTCC | 23 | |
| | gBlock R | ATACCGTGTG | | |
| 9 | Bs CDA5 | CTTCTGGAGGAGTCCGGGGCCAGATGCGTCC | 24 | Quickchange to make |
| | Y34A F | CTGTGC | | Bs-CDA5-Y34A |
| 10 | BS CDA5 | GCACAGGGACGCATCTGGCCCCGGACTCCTC | 25 | |
| | Y34A R | CAGAAG | | |
| 11 | pHT264 | GAGGAGggatccATGGTAAGTAAAGGAGAGG | 26 | Amplify Bs-CDA5 for |
| | BS CDA5 F | AGTTGTTC | | B. subtilis expression |
| 12 | pHT264 | GAGGACcccgggTTATTTATACAGTTCGTCC | 27 | |
| | BS CDA5 R | ATACCGTGTG | | |

TABLE 6
Plasmids.

| Plasmid | Name | Description | Source |
|---|---|---|---|
| 1 | pET28b- | E. coli expression | Sureka, K. et al. |
| | lmo0553 | vector for Lmo0553 | Cell 158, |
| | | | 1389-1401 (2014) |
| 2 | pet20b-DisA | E. coli expression | Sureka, K. et al. |
| | | vector for DisA | Cell 158, |
| | | | 1389-1401 (2014) |
| 3 | pSPEEDET- | E. coli expression | McFarland, A. P. |
| | mRECON | vector for RECON | et al. Immunity |
| | | | 46, 433-445 |
| | | | (2017) |
| 4 | pET28b- | E. coli expression | Witte, C. E. et al. |
| | PdeA(84-657) | vector for soluble PdeA | MBio 4, |
| | | | e00282-13 (2013 |
| 5 | pcDNA3-empty | Control transient | Gift |
| | vector | expression vector | |
| 6 | pcDNA4-DisA | Transient expression | Gift |
| | | vector for DisA | |
| 7 | pcDNA4-WspR* | Transient expression | Gift |
| | | vector for WspR* | |
| 8 | pcDNA3-cGAS | Transient expression | Gift |
| | | vector for cGAS | |
| 9 | pET15b-eCFP- | Vector for FRET cloning | Gift |
| | 12AA-eYFP | | |
| 10 | pET15b-CDA5 | E. coli expression | This study |
| | | vector for CDA5 | |
| 11 | pSLIK-CDA5 | Lentiviral expression | This study |
| | | vector for CDA5 | |
| 12 | pET15b-CDA5- | E. coli expression | This study |
| | Y34A | vector for CDA5-Y34A | |
| 13 | pET15b-Bs- | Subcloning vector for | This study |
| | CDA5 | Bs-CDA5 | |
| 14 | pET15b-Bs- | Subcloning vector for | This study |
| | CDA5-Y34A | BS-CDA5-Y34A | |
| 15 | pHT264 BS | B. subtilis expression | This study |
| | CDA5 | vector for CDA5 | |
| 16 | pHT264 BS | B. subtilis expression | This study |
| | CDA5 -Y34A | vector for CDA5- Y34A | |

TABLE 7
Strains.

| Number | Name | Description | Source |
|---|---|---|---|
| 1 | WT | B. subtilis strain 168 | Purchased |
| | | | from Bacillus |
| 2 | ΔdisA | B. subtilis strain 168 ΔdisA | Genetic Stock |
| | | | Center |
| 3 | ΔpgpH | B. subtilis strain 168 ΔpgpH | |

Protein Expression

To obtain L. monocytogenes full-length Lmo0553, pET28a-lmo0553 vector with an N-terminal hexa-histidine tag was transformed into BL21 Star (DE3) cells. The cells were cultured in LB medium with 35 mg/L kanamycin and were induced for 14 h at 20° C. with 1 mM IPTG. A selenomethionine-derivative of Lmo0553 was expressed using a methionine-auxotroph E. coli BL21 strain, and the defined medium was supplemented with selenomethionine (Hendrickson, W. A., et al. Selenomethionyl proteins produced for analysis by multiwavelength anomalous diffraction (MAD): a vehicle for direct determination of three-dimensional structure. EMBO J. 9, 1665-1672 (1990), incorporated herein by reference in its entirety). The protein was purified through nickel-agarose affinity chromatography followed by gel filtration chromatography (S-300, GE Healthcare). The purified protein was concentrated to 30 mg/mL in a buffer containing 20 mM Tris (pH 8.0), 250 mM NaCl, 5% (v/v) glycerol, and 5 mM dithiothreitol, flash-frozen in liquid nitrogen and stored at −80° C. The N-terminal hexa-histidine tag was not removed for crystallization.

To obtain CDA5, pET15b plasmids encoding CDA5 and CDA5-Y34A were transformed into BL21 Star (DE3) cells. An overnight culture of the transformed bacteria was inoculated into 1 L of LB broth and grown and grown at 37° C. until an OD₆₀₀ between 0.5-0.7 at which point protein expression was induced by the addition of 0.1 mM isopropyl P-D-1-thiogalactopyranoside (IPTG) for 16-20 hours at 18° C. The protein was purified using nickel-agarose affinity chromatography (Thermo Scientific). The protein was subsequently buffer exchanged (Cytiva) into Buffer A (40 mM Tris pH 7.5, 100 mM NaCl, 20 mM MgCl2, 1 mM DTT). Protein samples were tested for purity by SDS-PAGE followed by Coomassie Brilliant Blue staining. Samples were then flash-frozen in liquid nitrogen and stored at −80° C. until use in biochemical assays. DisA, RECON, and PdeA were purified the same as above with the exception that they were induced with 1 mM IPTG at 37° C. for 4 hours.

Protein Crystallization

Crystals of Lmo0553 in complex with c-di-AMP were grown by the sitting-drop vapor diffusion method at 20° C. The protein at 15 mg/mL was first incubated with 2.5 mM c-di-AMP for 30 min, and then mixed with reservoir solution containing 23% (w/v) PEG3350, and 0.2 M calcium acetate. The crystals were cryo-protected with the reservoir solution supplemented by 12% (v/v) glycerol and flash-frozen in liquid nitrogen for data collection at 100 K. Crystals of free Lmo0553 were grown by the sitting-drop vapor diffusion method at 20° C. The protein at 16 mg/mL was mixed with a reservoir solution containing 1.4 M ammonium sulfate, and 0.1 M sodium citrate (pH 5). The crystals were cryo-protected with 20% (v/v) glycerol and flash-frozen in liquid nitrogen for data collection at 100 K.

Data Collection, Structure Determination and Refinement

X-ray diffraction data for Lmo0553 were collected on the X29A beamline at the National Synchrotron Light Source. The diffraction images were processed using HKL2000 (Otwinowski, Z. & Minor, W. Processing of X-ray diffraction data collected in oscillation mode. in Methods in Enzymology vol. 276 307-326 (Academic Press, 1997), incorporated herein by reference in its entirety). The structure was solved using the single-wavelength anomalous dispersion (SAD) method with selenomethionine-derivatized crystals, using the program PHENIX (Adams, P. D. et al. PHENIX: building new software for automated crystallographic structure determination. Acta Crystallogr. D Biol. Crystallogr. 58, 1948-1954 (2002), incorporated herein by reference in its entirety). Manual rebuilding was carried out in Coot (Emsley, P. & Cowtan, K. Coot: model-building tools for molecular graphics. Acta Crystallogr. D Biol. Crystallogr. 60, 2126-2132 (2004), incorporated herein by reference in its entirety) and refinement was done with the program Refmac (Murshudov, G. N., et al. Refinement of macromolecular structures by the maximum-likelihood method. Acta Crystallogr. D Biol. Crystallogr. 53, 240-255 (1997), incorporated herein by reference in its entirety). Data collection and refinement statistics are summarized in TABLE 4.

Radioactive Nucleotide Binding Assays

[³²P] 3′3′-cyclic di-AMP was synthesized identically as described in Example 1 (see also Pollock, A. J., et al. A STING-based biosensor affords broad cyclic dinucleotide detection within single living eukaryotic cells. Nat. Commun. 11, 3533 (2020), incorporated herein by reference in its entirety). This nucleotide was then used to perform DRaCALA assays (Roelofs, K. G., et al. Differential radial capillary action of ligand assay for high-throughput detection of protein-metabolite interactions. Proc. Natl. Acad. Sci. U.S.A 108, 15528-15533 (2011), incorporated herein by reference in its entirety). Briefly, binding assays were performed in Buffer A at room temperature. To determine binding affinities, two-fold serial dilutions of proteins were incubated with ˜1 nM of radioactive 3′3′-cyclic di-AMP for 10 minutes then blotted onto nitrocellulose membranes and allowed to air dry. To determine binding specificity, samples were pre-incubated with 500 μM excess unlabeled nucleotides for 10 minutes followed by incubation with ˜1 nM of radioactive 3′3′-cyclic-di-AMP for 10 minutes then blotted onto nitrocellulose membranes and allowed to dry. Finally, to determine the time frame of competition, ˜1 nM of radioactive 3′3′-cyclic di-AMP was preincubated with protein for 10 minutes at which point 500 μM of unlabeled 3′3′-cyclic-di-AMP was added, mixed, and blotted onto nitrocellulose membranes every two seconds and allowed to dry. [³²P] radioactivity was visualized by exposure onto Phosphor-Imager screens, which were developed using a Typhoon FLA 9000 biomolecular imager (GE Healthcare).

In Vitro FRET Measurements

In all assays, 2 μM of CDA5 or CDA5-Y34A was incubated in black flat bottom half volume opaque 96-well plates (Greiner Bio-One). For nucleotide response assays, two-fold dilutions of 3′3′-c-di-AMP (Invitrogen) were made in molecular grade water and added to the protein solution. eCFP and FRET fluorescence was monitored at room temperature using a fluorimeter (BioTek Synergy H1 Hybrid Reader, Biotek Instruments) at 425 nm excitation and read at 480 nm and 535 nm emission wavelengths for eCFP and FRET respectively. PdeA enzyme activity assay was performed as above with the exception that: Buffer A was supplemented 20 mM MnCl₂, two fold dilutions of PdeA rather than c-di-AMP were added to each well, samples were spiked with 2 μM c-di-AMP at t=0 and t=90 minutes, and the assay was monitored every 5 minutes.

Eukaryotic CDA5 Specificity Assays

Assays were performed similarly as described in Example 1 (see also Pollock, A. J., et al. A STING-based biosensor affords broad cyclic dinucleotide detection within single living eukaryotic cells. Nat. Commun. 11, 3533 (2020), incorporated herein by reference in its entirety). Briefly, Human Embryonic Kidney (HEK) 293T cells were grown in Glutamax Dulbecco's Modified Eagle Medium (DMEM) (Gibco) supplemented with 10% (v/v) heat-inactivated FBS (HyClone) and 100 U mL⁻¹ penicillin and 100 μg mL⁻¹ streptomycin and maintained at 37° C. and 5% CO2 in a humidified incubator. Self-inactivating lentivirus was made via transfection of a semi-confluent 10 cm dish of HEK293T cells with 4 μg of psPAX2, 2 μg of pCMV-VSV-G, and 4 μg of pSLIK lentiviral vector using Poly(ethyleneimine) (PEI). Growth media was replaced 24 hours after transfection and supernatants collected at 48 and 72 hours, pooled, and filtered through a 0.45 μm filter. Lentivirus was then concentrated with a Lenti-X concentrator (Takara) and added to 4 million HEK293T cells seeded on a 10 cm plate and spinfected for 1 hour at 500×g. After a 24-hour recovery period, media was replaced and supplemented with 2 μg mL⁻¹ puromycin (Gibco). Transduced cells were continually passaged and maintained in selection media containing puromycin. For FRET measurements, 750,000 HEK293T cells transduced with the doxycycline inducible pSLIK-CDA5 plasmid were plated in a 6-well culture dish. The subsequent day, cells were transfected with indicated concentrations of cyclase-encoding plasmids using PEI transfection reagent. One hour after transfection, biosensor expression was induced by the addition of Doxycycline Hydrochloride (Sigma-Aldrich) at 2 μg mL⁻¹. The next day, cells were harvested via resuspension in room temperature PBS and CDA5 FRET measurements were collected by FACS analysis. Cells were analyzed using a LSR II flow cytometer (BD) with the following voltages: FSC-350 SSC-240 V500-350 Pacific Blue-420 GFP-400. Data was analyzed using FlowJo software (Tree Star).

Bacterial FRET Measurements

E. coli FRET measurements were obtained by transforming BL21 Star (DE3) cells with pET15b-CDA5 and pET15b-CDA5-Y34A in combination with either pBAVE-EV or pBAVE-DacA and plated on LB Carb 100 μg mL⁻¹ Kan 50 μg mL⁻¹ plates. An overnight culture of the transformed bacteria was inoculated into 1 L of LB broth and grown and grown at 37° C. until an OD₆₀₀ between 0.5-0.7 at which point protein expression was induced by the addition of 0.1 mM isopropyl β-D-1-thiogalactopyranoside (IPTG) for 16-20 hours at 18° C. At this point, cells were spun down and resuspended in room temperature PBS and CDA5 FRET measurements collected by FACS analysis. Cells were analyzed using a LSR II flow cytometer (BD) with the following voltages: FSC-400 SSC-200 V500-500 Pacific Blue-600 GFP-400. Data was analyzed using FlowJo software (Tree Star)

B. subtilis FRET measurements were obtained by transforming B. subtilis with pET264-CDA5 and pET264-CDA5-Y34A and plating cells on LB Carb 100 μg mL-1 plates and incubated at 37° C. Single colonies were then struck onto LB Carb 100 μg mL-1 IPTG 1 mM plates and incubated overnight at 30° C. The resulting single colonies were harvested in 10% LB media (FRET detectable up to 30% LB media) and clumps dissociated by passing cells 2-3 times through a 27 gauge needle. Cells were then grown shaking at 37° C. until desired time points at which CDA5 FRET measurements were collected by FACS analysis. Cells were analyzed using a LSR II flow cytometer (BD) with the following voltages: FSC-400 SSC-200 V500-450 Pacific Blue-550 GFP-375. Data was analyzed using FlowJo software (Tree Star).

Mass Spectrometry

The OD₆₀₀ of B. subtilis samples was taken. Then, half of the sample was analyzed by FACS analysis and the other half pelleted and frozen for c-di-AMP extraction. Cell pellets were resuspended in 50 μL of 0.5 μM heavy-labeled (C13 N15) c-di-AMP, then mixed with 500 μL of methanol and sonicated. The sample was pooled, centrifuged, and supernatant saved. The resulting pellet was resuspended in 50 μL water then mixed with 500 μL methanol and sonicated again. The solution was centrifuged and the second supernatant pooled with the first. The extract was then dried using a speed vacuum concentrator. The resulting film was resuspended in 50 μL of molecular grade water and measured by mass spectrometry as described (Huynh, T. N. et al. An HD-domain phosphodiesterase mediates cooperative hydrolysis of c-di-AMP to affect bacterial growth and virulence. Proc. Natl. Acad. Sci. U.S.A. 112, E747-56 (2015), incorporated herein by reference in its entirety).

While illustrative embodiments have been illustrated and described, it will be appreciated that various changes can be made therein without departing from the spirit and scope of the invention.

The embodiments of the invention in which an exclusive property or privilege is claimed are defined as follows:

1. A fusion protein comprising a cyclic dinucleotide binding domain having an N terminal end and a C terminal end, a first fluorescent domain, and a second fluorescent domain.

2. The fusion protein of claim 1, wherein the fusion protein further comprises a first linking domain linking the first fluorescent domain and the N terminal end of the cyclic dinucleotide binding domain.

3. The fusion protein of claim 1, wherein the fusion protein further comprises a second linking domain linking the C terminal end of the cyclic dinucleotide binding domain and the second fluorescent domain.

4. The fusion protein of claim 1, wherein the fusion protein further comprises a first linking domain linking the first fluorescent domain and the N terminal end of the cyclic dinucleotide binding domain, wherein the fusion protein further comprises a second linking domain linking the C terminal end of the cyclic dinucleotide binding domain and the second fluorescent domain, and wherein the first linking domain comprises 7 amino acids and the second linking domain comprises 2 amino acids.

5. The fusion protein of claim 1, wherein the fusion protein further comprises a first linking domain linking the first fluorescent domain and the N terminal end of the cyclic dinucleotide binding domain, wherein the fusion protein further comprises a second linking domain linking the C terminal end of the cyclic dinucleotide binding domain and the second fluorescent domain, and wherein the first linking domain comprises 2 amino acids and the second linking domain comprises 2 amino acids.

6. The fusion protein of claim 1, wherein the first fluorescent domain and the second fluorescent domain are complementary components of a luminescent protein.

7. The fusion protein of claim 1, wherein the first fluorescent domain and the second fluorescent domain form a FRET pair.

8. The fusion protein of claim 7, wherein the FRET pair is a blue/orange FRET pair, a cyan/yellow FRET pair, or a far-red FRET pair.

9. The fusion protein of claim 7, wherein the fusion protein generates a FRET signal upon binding a cyclic dinucleotide to the cyclic dinucleotide binding domain.

10. The fusion protein of claim 1, wherein the first fluorescent domain and the second fluorescent domain form a BRET pair.

11. The fusion protein of claim 1, wherein the cyclic dinucleotide binding domain comprises an amino acid sequence derived from a cyclic dinucleotide binding domain of a stimulator of interferon (IFN) genes (STING) protein.

12. The fusion protein of claim 11, wherein the cyclic dinucleotide binding domain comprises an amino acid sequence derived from the cyclic dinucleotide binding domain of a murine STING (mSTING) protein or comprises an amino acid sequence with at least about 90% identity to SEQ ID NO:33.

13. The fusion protein of claim 11, wherein the cyclic dinucleotide binding domain of the fusion protein binds a cyclic dinucleotide or ligand of a wild-type human or murine STING protein.

14. The fusion protein of claim 13, wherein the cyclic dinucleotide is one or more of 2′,3′-cyclic GMP AMP (2′,3′-cGAMP), 3′,3′-cyclic di-AMP, 3′,3′-cyclic di-GMP, 3′,3′-cyclic GMP AMP (3′,3′-cGAMP), or a synthetic cyclic dinucleotide.

15. The fusion of claim 1, the cyclic dinucleotide binding domain comprises an amino acid sequence derived from a cyclic dinucleotide binding domain of Listeria monocytogenes c-di-AMP effector protein (Lmo0553) or comprises an amino acid sequence with at least about 60% identity to SEQ ID NO:35.

16. The fusion of claim 15, wherein the cyclic dinucleotide is 3′3′-cyclic-di-AMP.

17. A polynucleotide encoding at least one fusion protein according to claim 1.

18. A cell comprising a polynucleotide of claim 17.

19. A method for detecting a cyclic dinucleotide in a solution, comprising
contacting the solution comprising a cyclic dinucleotide with a fusion protein of claim 1 for a time sufficient for the cyclic dinucleotide to bind to the cyclic dinucleotide binding domain, and
detecting a signal generated by the fusion protein.

20. A method for detecting a cyclic dinucleotide in a cell, comprising expressing a fusion protein of claim 1 in a cell and detecting a signal generated by the fusion protein.


SEQUENCE LISTING (36 sequences)

SEQ ID NO: 1 - DNA, 33 bp, Artificial sequence
  Synthetic
gaggagactagtttaaatgttgcccacgggctg

SEQ ID NO: 2 - DNA, 33 bp, Artificial sequence
  Synthetic
gaggagggtaccttcctgacgaatgtgccggag

SEQ ID NO: 3 - DNA, 48 bp, Artificial sequence
  Synthetic
gaggagactagtggcggatccgggggcttaaatgttgcccacgggctg

SEQ ID NO: 4 - DNA, 48 bp, Artificial sequence
  Synthetic
gaggagggtaccgcccccggatccgccttcctgacgaatgtgccggag

SEQ ID NO: 5 - DNA, 39 bp, Artificial sequence
  Synthetic
ggcatcaagaatcgggtttcttccaacagcgtctacgag

SEQ ID NO: 6 - DNA, 39 bp, Artificial sequence
  Synthetic
ctcgtagacgctgttggaagaaacccgattcttgatgcc

SEQ ID NO: 7 - DNA, 35 bp, Artificial sequence
  Synthetic
ctgtatcctggagtacgccgcccccttgcagaccc

SEQ ID NO: 8 - DNA, 35 bp, Artificial sequence
  Synthetic
gggtctgcaagggggcggcgtactccaggatacag

SEQ ID NO: 9 - DNA, 38 bp, Artificial sequence
  Synthetic
cccagcaaaacatcgaccatgctggcatcaagaatcgg

SEQ ID NO: 10 - DNA, 38 bp, Artificial sequence
  Synthetic
ccgattcttgatgccagcatggtcgatgttttgctggg

SEQ ID NO: 11 - DNA, 39 bp, Artificial sequence
  Synthetic
ccccagcaaaacatcgacgctgctggcatcaagaatcgg

SEQ ID NO: 12 - DNA, 39 bp, Artificial sequence
  Synthetic
ccgattcttgatgccagcagcgtcgatgttttgctgggg

SEQ ID NO: 13 - DNA, 41 bp, Artificial sequence
  Synthetic
tgatcactagcgtacgaccatgggcagcagccatcatcatc

SEQ ID NO: 14 - DNA, 37 bp, Artificial sequence
  Synthetic
tcttccaattcgtacgtcatccgccaaaacagccaag

SEQ ID NO: 15 - DNA, 65 bp, Artificial sequence
  Synthetic
tcttccaattcgtacgtcagtccaacttgaccctcttggcagcaggtccgccaaaacagccaagc

SEQ ID NO: 16 - DNA, 56 bp, Artificial sequence
  Synthetic
gaggagactagtatgttgataaaaaacctttgtatccctaaaataaatttaacaac

SEQ ID NO: 17 - DNA, 35 bp, Artificial sequence
  Synthetic
gaggagggtaccattccagccatcttggagcaaac

SEQ ID NO: 18 - DNA, 41 bp, Artificial sequence
  Synthetic
tgatcactagcgtacgaccatggttagcaagggagaggagc

SEQ ID NO: 19 - DNA, 52 bp, Artificial sequence
  Synthetic
tcttccaattcgtacgttacttataaagctcatccattccatgtgtaattcc

SEQ ID NO: 20 - DNA, 53 bp, Artificial sequence
  Synthetic
ggctatccatttacttgaagaatctggtgctcgctgtgttcctgtattagacg

SEQ ID NO: 21 - DNA, 53 bp, Artificial sequence
  Synthetic
cgtctaatacaggaacacagcgagcaccagattcttcaagtaaatggatagcc

SEQ ID NO: 22 - DNA, 42 bp, Artificial sequence
  Synthetic
gaggagggcggccgcatggtaagtaaaggagaggagttgttc

SEQ ID NO: 23 - DNA, 41 bp, Artificial sequence
  Synthetic
gaggaggcggccgcctttatacagttcgtccataccgtgtg

SEQ ID NO: 24 - DNA, 37 bp, Artificial sequence
  Synthetic
cttctggaggagtccggggccagatgcgtccctgtgc

SEQ ID NO: 25 - DNA, 37 bp, Artificial sequence
  Synthetic
gcacagggacgcatctggccccggactcctccagaag

SEQ ID NO: 26 - DNA, 39 bp, Artificial sequence
  Synthetic
gaggagggatccatggtaagtaaaggagaggagttgttc

SEQ ID NO: 27 - DNA, 41 bp, Artificial sequence
  Synthetic
gaggagcccgggttatttatacagttcgtccataccgtgtg

SEQ ID NO: 28 - protein, 5 aa, Artificial sequence
  Synthetic
GGSGG

SEQ ID NO: 29 - protein, 230 aa, Artificial sequence
  Synthetic
ETTMGVIKPDMKIKLKMEGNVNGHAFVIEGEGEGKPYDGTNTINLEVKEGAPLPFSYDILTTAFAYGNRAFTKYPDDIPNYFKQSFPEGYSWERTMTFEDKGIVKVKSDISMEEDSFIYEIHLKGENFPPNGPVMQKKTTGWDASTERMYVRDGVLKGDVKHKLLLEGGGHHRVDFKTIYRAKKAVKLPDYHFVDHRIEILNHDKDYNKVTVYESAVARNSTDGMDELYK

SEQ ID NO: 30 - protein, 219 aa, Artificial sequence
  Synthetic
VSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQMKTTYKAAKEILEMPGDHYIGHRLVRKTEGNITEQVEDAVALEKL

SEQ ID NO: 31 - protein, 239 aa, Artificial sequence
  Synthetic
MVSKGEELFGGIVPILVELEGDVNGHKFSVSGEGEGDATYGKLTLKFICTTGKLPVPWPTLVTTLTWGVQCFSRYPDHMKQHDFFKSVMPEGYVQERTIFFKDDGNYKTRAEVKFEGDTLVNRIELKGIDFKEDGNILGHKLEYNYISHNVYITADKQKNGIKANFKARHNITDGSVQLADHYQQNTPIGDGPVILPDNHYLSTQSKLSKDPNEKRDHMVLLEFVTAAGITHGMDELYK

SEQ ID NO: 32 - protein, 239 aa, Artificial sequence
  Synthetic
MVSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLFKDPNEKRDHMVLLEFLTAAGITEGMNELYK

SEQ ID NO: 33 - protein, 184 aa, Mus musculus
LNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQE

SEQ ID NO: 34 - protein, 643 aa, Artificial sequence
  Synthetic
EETTMGVIKPDMKIKLKMEGNVNGHAFVIEGEGEGKPYDGTNTINLEVKEGAPLPFSYDILTTAFAYGNRAFTKYPDDIPNYFKQSFPEGYSWERTMTFEDKGIVKVKSDISMEEDSFIYEIHLKGENFPPNGPVMQKKTTGWDASTERMYVRDGVLKGDVKHKLLLEGGGHHRVDFKTIYRAKKAVKLPDYHFVDHRIEILNHDKDYNKVTVYESAVARNSTDGMDELYKTSGGSGGLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTDGNSFSLSQEVLRHIRQEGTVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQMKTTYKAAKEILEMPGDHYIGHRLVRKTEGNITEQVEDAVALEKL

SEQ ID NO: 35 - protein, 127 aa, Listeria monocytogenes
MLIKNLCIPKINLTTVPGDATLQEAIHLLEESGYRCVPVLDEKGEKFLGNIYKMHIYKHAANGGSLSDPVMSLIKNATKHICVNASFFEVFFTIKELPYIAVLNEQGNFYGILTHGKLLGLLQDGWN

SEQ ID NO: 36 - protein, 609 aa, Artificial sequence
  Synthetic
MVSKGEELFTGVVPILVELDGDVNGHKFSVSGEGEGDATYGKLTLKLLCTTGKLPVPWPTLVTTLGYGVQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLFKDPNEKRDHMVLLEFLTAAGITEGMNELYKTSMLIKNLCIPKINLTTVPGDATLQEAIHLLEESGYRCVPVLDEKGEKFLGNIYKMHIYKHAANGGSLSDPVMSLIKNATKHICVNASFFEVFFTIKELPYIAVLNEQGNFYGILTHGKLLGLLQDGWNGTMVSKGEELFGGIVPILVELEGDVNGHKFSVSGEGEGDATYGKLTLKFICTTGKLPVPWPTLVTTLTWGVQCFSRYPDHMKQHDFFKSVMPEGYVQERTIFFKDDGNYKTRAEVKFEGDTLVNRIELKGIDFKEDGNILGHKLEYNYISHNVYITADKQKNGIKANFKARHNITDGSVQLADHYQQNTPIGDGPVILPDNHYLSTQSKLSKDPNEKRDHMVLLEFVTAAGITHGMDELYK